(19) United States
(12) Patent Application Publication
(10) Pub. No.: US 2003/0215936 A1
(43) Pub. Date: Nov. 20, 2003

(54) High-throughput tissue microarray technology and applications

(76) Inventors: Olli Kallioniemi, Rockville, MD (US); Guido Sauter, Basel (CH); Stephen B. Leighton, Silver Springs, MD (US); Juha Kononen, Arlesheim (CH); Thomas J. Pohida, Monrovia, MD (US); John William Karareka, Rockville, MD (US); Ghadi Hamdi Salem, College Park, MD (US)

Correspondence Address: KLARQUIST SPARKMAN, LLP, 121 SW SALMON STREET, SUITE 1600, PORTLAND, OR, 97204, US

(21) Appl. No.: 10/149,520

(22) PCT Filed: Dec. 13, 2000
(86) PCT No.: PCT/US00/34043

(51) Int. Cl. C12M 1/34; G01N 1/30; G01N 33/48

(52) U.S. Cl. 435/287.1; 435/40.5

(57) ABSTRACT

A method and apparatus are disclosed for a high-throughput, large-scale molecular profiling of tissue specimens by retrieving a donor tissue specimen from an array of donor specimens, placing a sample of the donor specimen in an assigned location in a recipient array, providing substantial copies of the array, performing a different biological analysis of each copy, and storing the results of the analysis. The results may be compared to determine if there are correlations or discrepancies between the results of different biological analyses at each assigned location, and also compared to clinical information about the human patient from which the tissue was obtained. The results of similar analyses on corresponding sections of the array can be used as quality control devices, for example by subjecting the arrays to a single simultaneous investigative procedure. Uniform interpretation of the arrays can be obtained, and compared to interpretations of different observers.

### TECHNICAL FIELD

This invention generally relates to the microscopic, histologic and/or molecular analysis of tissue or cellular specimens and, more particularly, to the construction of tissue microarrays for holding multiple tissue specimens and the use of such tissue microarrays for high-throughput molecular analyses, as well as didactic and quality control purposes.

### BACKGROUND

Microscopic examination of tissue specimens has helped clarify biological disease mechanisms. In standard histopathology, a diagnosis is made on the basis of cellular morphology and staining characteristics. This approach has improved disease diagnosis and classification, and promoted development of effective medical treatments for a variety of illnesses, such as cancer. However, cellular morphology reveals only a limited amount of information regarding the molecular mechanisms of disease.

Recently, several techniques have evolved to explore molecular and cellular disease mechanisms. For example, the biological behavior of some cancers may be predicted by certain genetic abnormalities (such as mutations in certain oncogenes; Faderl et al., N. Engl. J. Med. 341: 164-172 (1999)), expression of hormonal receptors (such as estrogen receptor expression in breast cancer; Eisen and Weber, Current Opinion in Oncology 10:486-91 (1998)), or the abnormal expression of tumor-associated cell surface proteins (such as neural cell adhesion molecule expression in neuroendocrine lung tumors; Lantuejoul et al., Am. J. Surg. Pathol. 22:1267-1276 (1998)). These abnormalities may be assessed by examining tissue specimens with techniques such as immunohistochemistry, in situ hybridization, and DNA amplification using the polymerase chain reaction (PCR). The information thus gained may be used to determine an individual's prognosis and likelihood of response to therapy. It is also useful for understanding the fundamental molecular and cellular mechanisms of human disease.

New and important molecular disease markers, and a better understanding of human disease processes, may result from improved methods for evaluating histopathology, genetic abnormalities, and gene expression in large numbers of tissue specimens. However, there has been only limited development of such methods. The lack of progress can be attributed in part to the difficulties involved in preparing multiple tissue specimens for analysis. Multiple tissue specimens have been assembled using manual methods, but these methods are labor-intensive, time-consuming, and inefficient. See, e.g., Wan et al., Journal of Immunological Methods 103:121-129 (1987); Furmanski et al., U.S. Pat. No. 4,914,022; Battifora and Mehta, Lab. Invest. 63:722-724 (1990), and U.S. Pat. No. 5,002,377. Such limitations render existing assembly methods inadequate for rapid parallel analysis of a variety molecular markers in a large number of different tissues.

High throughput methods are now being developed for analysis of gene expression in tissue extracts. Microarrays of DNA sequences are printed on a solid support surface using computer-controlled, high-speed robotics. These DNA microarrays typically include representative sequences from genes of interest. Total mRNA is isolated from a tissue sample using standard techniques, and reverse transcribed in the presence of a fluorescence-tagged deoxyribonucleotide. The fluorescent mixture of total cellular cDNA is then hybridized to the microarray, and fluorescence intensity quantified by laser confocal scanning microscopy and image analysis. See Schena et al., Science 270: 467-470, 1995; Schena, BioEssays 18: 427-431, 1996; Soares, Current Opinion in Biotechnology 8:542-546, 1997; Ramsay, Nature Biotechnology 16: 14-44, 1998; Service, Science 282: 396-399, 1998; U.S. Pat. No. 5,700,637. Alternatively, the microarray may be constructed using genomic DNA or cDNA from one or more tissues, and detection accomplished using fluorescence-tagged oligonucleotides containing representative sequences from genes of interest. See Schena et al., BioEssays 18: 427-431, 1996.

An important medical goal is to validate, prioritize and further study genes and proteins discovered in large-scale molecular surveys as well as to establish the diagnostic, prognostic and therapeutic importance of a rapidly increasing number of disease candidate genes. This in turn will require rapid analysis of hundreds or thousands of specimens from patients in different stages of disease, with minimal requirement for operator intervention. To date, however, there has been limited progress in automating analysis of tissue samples. As noted, available manual methods for assembly of tissue specimens (such as those described by Wan et al., Furmanski et al., and Battifora and Mehta) are labor-intensive and inefficient. Bolles, U.S. Pat. No. 5,746,855, teaches an apparatus and method for automatic archival storage of tissue sections after they are cut from a sample block. A section of adhesive tape is applied to the sample block prior to cutting a section with a microtome; after the section is cut, the adhesive tape is automatically lifted, advanced, and pressed to a microscope slide containing a stronger adhesive material. Bernstein et al., U.S. Pat. Nos. 5,355,439 and 5,930,461 teach a method and apparatus for automated tissue assay, wherein a processor directs a robotic arm to move tissue samples between multiple workstations. Each workstation performs a different step in a biological test or analysis, e.g., tissue fixation, binding of a particular antibody, washing, with the processor ensuring that the step is appropriately timed. While the Bolles and Bernstein et al. teachings reduce the amount of operator intervention necessary for tissue sectioning and staining, they do not address the many other problems associated with high-throughput analysis of large numbers of tissue samples.

Achieving the goal of establishing the diagnostic, prognostic and therapeutic importance of disease candidate genes has also been slowed by inconsistencies in analysis. Up until the present time, analysis has been performed by many different researchers, at different locations. This approach has produced discordant results, that have slowed the progress of medical research. These discordant results are influenced by the presence of many different variables, such as differences in the biological material (such as tumor samples) that are obtained from different patients, the length of time before fixation, varying techniques used for fixation and antigen retrieval, differences in antibodies/probes which are selected by different researchers, variations in staining or hybridization, and interpretation of the results of such bioanalyses by different observers. Because of these multiple variables, numerous confirmatory studies are often required to obtain a sufficiently large number of results to compensate for these variables. Meta-analyses of multiple different studies can average out such variabilities, but the requirement for such studies is expensive and time-consuming, and slows the progress of medical research.

The second problem is that using conventional sectioning of tissue specimens, only a very limited number of molecular analyses can be performed per tissue. Typically, using 5 micrometer sections, one can only cut about 300 sections from each tissue block, and thereby carry out 300 different molecular analyses. There are over 60,000 genes in the human genome, and for each gene or gene product, multiple probes and antibodies can be generated. Therefore, only a very small fraction of all interesting genes/proteins can be analyzed from a set of valuable clinical specimens.

A related problem with tissue examination is that it is often subject to variable interpretation by different examiners. Pathologic examination (including molecular analysis) is usually accomplished by microscopic examination of biological material by a clinician or researcher. When the clinician is a pathologist, important clinical decisions are often made based on an interpretation of the biological material. For example, if a bladder cancer specimen is judged to show a grade 3 (poorly differentiated) bladder tumor, the patient's bladder is often removed (cystectomy) because large scale studies have shown such surgery to be required to provide the greatest chance of survival. However, if the tissue is judged to show a grade 2 tumor (moderately differentiated) more conservative measures are adopted which would be inappropriate for more advanced disease. Since the selection of an appropriate treatment requires that pathologic diagnoses be made in accordance with uniform standards, methods are needed to help ensure that clinicians in different localities have uniform standards of histologic diagnosis.

Advances in molecular medicine have further demonstrated the drawbacks of an absence of uniform standards for diagnosis. For example, Her-2 immunostaining results may determine whether a patient will undergo HERCEPTIN treatment. Despite the importance of a correct determination about the presence or absence of Her-2 immunostaining, there is still substantial inter-observer variation about the results of this test, and other molecular diagnostic assays. Since each molecular analysis is carried out on a different slide, multiple reasons may cause the variability. Often it remains impossible to identify the sources of this variability.

A related problem is that the training of pathologists and other trainees usually requires examination of a large number of many different tissue specimens, showing a spectrum of normal and diseased tissue. This has traditionally been accomplished by providing many mounted tissue sections which are examined through a microscope by the trainee. The trainee makes a histologic diagnosis, which is then compared to a histologic diagnosis made by a more experienced person (such as an expert pathologist).

The administration of examinations to large numbers of trainees (such as medical students and pathology residents) would also be facilitated by the availability of large numbers of specimens that have been subjected to analysis by a single expert, or a panel of experts whose results could be combined to provide a definitive diagnosis.

### SUMMARY OF THE DISCLOSURE

A method and apparatus are disclosed for a high-throughput, large-scale molecular profiling of tissue specimens by retrieving a donor tissue specimen from an array of donor specimens, placing a sample of the donor specimen in an assigned location in a recipient array, providing substantial copies of the array, performing the same or a different biological analysis of each copy, and storing and analyzing the results. In one embodiment, the substantial copies are formed by placing elongated sample cores from different donor specimens in a three-dimensional matrix, and cutting sections from the matrix to form multiple copies of a two-dimensional array mounted on a solid support such as a microscope slide. The copies can then be prepared or processed independently and subjected to different biological analyses. Preparation of the copies for biological analysis, and the biological analysis itself, may be done by automated, computer implemented means. The results of the different biological analyses may be stored in a database and compared to determine if there are correlations or discrepancies between the results of different biological analyses at each assigned location, and also compared to clinical information about the human patient from which the tissue was obtained.

The arrays can be used to make large numbers of tissue samples from pathology archives readily available for molecular analyses. One can also rapidly obtain information about the biological significance of biological markers (such as immunohistochemical markers and/or gene alterations) in a large number of specimens. One can acquire information about the localization of the biomolecule in different tissue and cell types (e.g. nuclear, cytoplasmic, membranous etc.). The results of similar analyses on corresponding sections from a set of reference/test/quality control specimens can be used as quality control devices, for example by subjecting all these arrays to a single simultaneous investigative procedure. This may help to substantially standardize molecular analyses, including uniform interpretation of the array data by different observers.

As is apparent from the foregoing, the present invention includes many different advantages and permutations. The foregoing and other features and advantages of the invention will become more apparent from the following detailed description of disclosed embodiments which proceeds with respect to the accompanying drawings.

### BRIEF DESCRIPTION OF THE FIGURES

FIG. 1 is a top view of two donor blocks, with a tissue specimen in each donor block, and showing locations from which tissue samples are punched from each of the tissue specimens.

FIG. 2 is a schematic view illustrating that multiple tissue samples are obtained from multiple tissue specimens (such as different tumors), and the samples from the different specimens are inserted into a recipient block in a three dimensional array. The array block is subsequently sectioned to produce multiple similar sections having samples from a particular specimen at a corresponding assigned location in all the array sections (as shown by the sample with diagonal hatch marks from the specimen of FIG. 1 in FIG. 2). Each of the sections may subsequently be subjected to the same or a different bioanalysis.

FIG. 3 is a schematic view illustrating a particular example in which a set of 1000 tissues (such as tumors) are sampled to a set of tissue microarray blocks. Each original tumor (measuring 15×15 mm) can be punched 324 times to produce 324 different recipient tissue microarray blocks. Each of the 324 recipient array blocks contains one specimen from all 1000 tumors. The tissue microarray block can be cut into 300 replicate sections. Since there are 324 of these replicate blocks, one can obtain up to 97,200 replicate sections, each of which contains 1000 different tumor samples, and each of the sections can be subjected to a different bioanalysis.

FIG. 4 illustrates digital images of tissue microarrays that can be stored in databases. On the left is one tissue microarray cross-section stained with one antibody. On the right are multiple images from one tumor arrayed in a single tissue microarray. The consecutive sections of this microarray have been serially analyzed with different antibodies and images of this one tumor at different sections (different stains) are depicted.

FIGS. 5A, 5B, 5C and 5D are schematic views illustrating an example of parallel analysis of arrays obtained by the method of the present invention.

FIG. 6 is an enlarged view of a portion of FIG. 5.

FIG. 7 is a top schematic view of a system for automated, high-speed fabrication of tissue microarrays in accordance with one embodiment of the present invention.

FIG. 8 is a perspective view of a portion of the system shown in FIG. 7, showing a storage station for tissue blocks.

FIG. 9 is a perspective view of a portion of the system shown in FIG. 7.

FIGS. 10A, 10B and 10C are top, perspective and side views of a tissue donor block in a carrier, which also illustrates a computer readable bar code label on the carrier.

FIG. 11 is an enlarged front view of the storage station of FIG. 13, illustrating the carriers inserted in the storage station.

FIG. 12 is an enlarged, fragmentary side view of the carrier held by a transporter.

FIG. 13 is a schematic illustration of a subsystem for locating and marking donor blocks.

FIG. 14 is a schematic illustration of a digital camera and bar code marking device.

FIG. 15 is a schematic view of a system processor for an image processor subsystem.

FIGS. 16-20 illustrate steps in the preparation of multiple tissue microarrays from the recipient block.

FIG. 21 is a schematic diagram of a computer system in which the method of the present invention can be implemented.

FIGS. 22A and 22B are schematic illustrations of the ability of the present invention to provide an entire pathology archive in a tissue microarray format that is readily available for molecular analyses.

FIGS. 23A and 23B schematically illustrate how the arrays can provide a comprehensive analysis of a molecular marker in a group of tissue specimens (such as different tumors) at the population level, instead of at the level of an individual tumor specimen.

FIG. 24 is a drawing which schematically illustrates use of the arrays as controls, for example in which the array contains normal tissues, positive controls, fixation controls, or tumors with known clinical outcomes. The inclusion of such controls in multiple different arrays that are constructed allows better comparison of results obtained at different time-points, for example by different investigators or centers.

FIG. 25 is a drawing which schematically illustrates the use of the arrays as quality control devices, in which different array sections are subjected to different procedures (for example by a different manufacturer) for subsequent comparison by other users of the procedure. This allows a determination as to whether different results obtained in the different centers are influenced by the reagents they use.

FIG. 26 is a drawing which schematically illustrates how the arrays can be used to improve quality control and enhance the pace of biological discovery by obtaining tissue specimens from multiple different researchers or centers, and combining the different specimens into a single array for simultaneous bioanalysis under substantially uniform conditions. This allows comparison whether specimens from different centers produce identical results (different results may arise e.g. from fixation differences).

FIG. 27 is a drawing which schematically illustrates how staining variability can be tested by having consecutive, essentially identical, sections of a single tissue microarray subjected to the same bioanalysis at different research centers. Variations in the stain (such as an IHC stain) can then be assigned to be dependent on the application of the bioanalysis or interpretation of the bioanalysis at these different centers.

FIG. 28 is a drawing which schematically illustrates that a tissue microarray which is prepared, sectioned and stained at a single location, can be disseminated to multiple observers, so that observer interpretations are based on a single substantially uniform array. This enables one to test how much variability there is in the interpretation of the same staining results by different observers. This also indicates how different, essentially identical sections could be used to train users to interpret tissue microarray slides.

FIG. 29A is a drawing which schematically illustrates reference points embedded in a tissue donor block, and FIG. 29B illustrates the use of those reference points in finding a region of interest in a tissue sample.

### DETAILED DESCRIPTION OF SEVERAL ILLUSTRATIVE EMBODIMENTS

Constructing tissue microarrays represents a considerably more complex problem than constructing nucleic acid microarrays. This problem is addressed by the present invention, in which one or multiple tissue samples are taken from a larger tissue specimen, and the samples are placed in corresponding positions of multiple recipient substrates.

Multiple tissue samples may be taken from multiple such tissue specimens, and the multiple samples from a particular specimen are similarly placed at corresponding positions in the multiple recipient substrates. Each of the resulting substrates contains an array of tissue samples from multiple specimens, in which corresponding positions in each of the arrays represent tissue samples from the same tissue specimen. In particular examples, each substrate is then sectioned into multiple similar sections with samples from the same tissue specimen at corresponding positions of the sequential sections. The different sections may then be subjected to different reactions, such as exposure to different histological stains or molecular markers, so that the multiple “copies” of the tissue microarrays can be compared for the presence of reactants of interest. The large number of tissue samples, which are repeated in each of a potentially large number of sections of multiple substrates, can be exposed to as many different reactions as there are sections. For example, about 100,000 array sections may be obtained from a set of 1000 tissue specimens measuring 15×15×3 mm. This approach provides a high-throughput technique for rapid parallel analysis of many different tissue specimens.

Also disclosed herein are particular examples of methods and apparatus for high-throughput large-scale molecular profiling of tissue specimens, in a manner that allows rapid parallel analysis of biological characteristics, such as molecular and cellular characteristics (for example, gene dosage or gene/protein expression), from hundreds of tissue specimens. In particular embodiments, the invention includes an automated apparatus for constructing tissue sample arrays from a plurality of specimens, in which the apparatus includes a specimen source from which tissue specimens are retrieved from assigned locations, a retriever that retrieves the tissue specimens from the specimen source, and a constructor that removes tissue samples from a plurality of the tissue specimens, and arrays the tissue samples at identifiable locations in three dimensional arrays in one or more substrates, wherein the different identifiable locations correspond to tissue samples from different tissue specimens.

In some embodiments, a sectioner then sections the three dimensional arrays into cut sections which carry the tissue samples from different tissue specimens, such that the locations in the three dimensional arrays correspond to locations in the cut sections. Some embodiments of the automated apparatus also include a controller that directs the retriever, constructor and sectioner, and can also record an identification of a subject associated with a particular specimen, as well as the identifiable locations in the three dimensional arrays and the cut sections that contain samples from that particular specimen.

Some embodiments of the apparatus include a donor source containing a plurality of identifiable donor tissue specimens, a retriever that retrieves the donor tissue specimens from the donor source, and a tissue microarray constructor that receives donor tissue samples from different tissue specimens retrieved by the retriever, and inserts the tissue samples into recipient blocks, thereby constructing a tissue microarray. A controller operates the retriever and array constructor, and identifies tissue samples within the array by recognizing identifiers associated with the tissue specimens. In particular embodiments, the tissue specimens are associated with a carrier medium, such as tissue block medium, and the apparatus further comprises a locator that records a location of the tissue specimen in the carrier medium, and a sectioner that cuts sections of the block.

In particular examples, the donor source is a donor specimen storage station, from which the constructor obtains tissue samples for insertion into the array, and to which the tissue specimens can be returned after obtaining tissue samples for insertion into the array. The tissue specimens can be located in the storage station by a coordinate positioning device, such as a robotic arm that retrieves tissue specimens from the donor source, and subsequently transfers tissue specimens to the tissue microarray constructor and returns tissue specimens to the donor source. A holder can be positioned to hold a separate tissue specimen and recipient block, and a reciprocal punch can be used to form receptacles in the recipient block and punch tissue samples from the tissue specimen. The punch then delivers a tissue sample from the tissue specimen to an identifiable receptacle in the recipient block. In disclosed embodiments, the recipient block is incrementally advanced to align a predetermined receptacle with the reciprocal punch, and deliver the tissue sample into the receptacle. A recorder records the location of the receptacle in the recipient block, and an identity of the tissue specimen from which the sample in the receptacle was obtained.

The apparatus can also include a microscope for locating a structure or region of interest (ROI) in a reference slide aligned with the tissue specimen prior to sampling, so that samples can be taken from the structure or region of interest. Moreover, once the samples have been placed in the recipient blocks, the blocks may be stored at identifiable locations in a donor source, such as an array of recipient blocks in a storage station. The same or a different storage station can also hold donor tissue specimens, prior and subsequent to taking the samples from the tissue specimens.

An advantage of some embodiments of the invention is that the cut images can be processed in a processing station, for example by exposing different sections to different biological reagents (such as standard stains, or immunohistochemical or genetic markers) that recognize biological structures in the cut sections. An imager then obtains an image of the cut processed sections, and an image processor identifies regions of the cut sections that contain images of biological interest (such as evidence of gene copy numbers), and stores images of the cut sections. If desired, quantities of biological reagents can be detected to quantify reactions (such as an amount of probe that hybridizes to the specimen as an indication of gene amplification or deletion), or to determine the distribution of the reagent in the sample.

The results of the image processing of any tissue microarrays can correlate the biological reactions of interest with identifying information about the cut sections and the subjects from whom the tissue specimens were obtained (such as clinical information about the subject). This information can be stored, for example, in a database that also includes the location of tissue donor specimens in the donor source, the location of recipient blocks in the recipient array, and the location of the tissue samples in the tissue microarray. Information in this sample database can be linked with information on the clinical, histological and demographic information of the patients.

In yet another embodiment, the apparatus for assembling tissue microarrays includes a donor specimen station which includes compartments for assigned tissue specimens, a computer readable identifier which identifies the tissue specimens in the donor specimen station, a donor block scanner for reading the identifers and locating the tissue specimens in the carrier, and a tissue microarray fabricator which obtains a plurality of elongated tissue samples from a plurality of tissue specimens and places them in a recipient block. The apparatus can also include a sectioner that sections the recipient block sufficiently transverse to the elongated tissue samples to form a series of block sections which retain a relationship of the elongated tissue samples in the recipient block, so that the sections from the same block are similar copies of one another. A processing station can then expose different similar sections to different biological markers that associate with biological substrates of interest in the sections, if the biological substrates are present, so that multiple tests can be simultaneously performed on multiple samples in multiple sections. In some examples, an automated scanner then scans the different sections to detect the presence of the biological markers in the different sections. A scanner can, for example, acquire images for a pathologist to interpret, or process the images to derive intensity information and save them for future use. A controller (such as a computer) can be programmed to perform these functions.

Although the donor specimen station, donor block scanner, tissue microarray fabricator, sectioner, processing station, automated scanner, controller, and other components of this system are described in combination, the invention also includes any of these sub-units in isolation, or in combination with any other sub-units. The sub-units need not be in the same physical location, nor do they need to run simultaneously. For example, arrays can be formed and then delivered to a sectioner, where sectioning is performed as a temporally unrelated step. Similarly, the array blocks may be sent to different facilities for sectioning and analysis, or the sections can be sent to different facilities for analysis. Data from off-site analyses can be sent back to a central database for storage and/or data analysis.

The disclosure also includes a method for performing molecular analysis of biological specimens by providing multiple sections each including multiple biological samples. In particular embodiments, subsets of the sections include multiple similar sections in which tissue samples from the same specimen are located at corresponding positions in different sections. The different sections are exposed to biological reagents (for example, different biological reagents) that react with biological substrates of interest in the biological samples, and images are obtained of the different sections after exposing the sections to the biological reagents. The images are then analyzed to determine whether a reaction with a substrate has occurred in the different sections, or specimen samples represented in the sections. The images also can be used to quantitate the degree of staining analyze its homogeneity within and between tissue samples, as well as determine the subcellular distribution of the biomolecules of interest.

In particular embodiments, the different biological specimens are obtained from different specimens (such as tumors, normal tissue, or biopsy specimens), and in particular examples the different specimens are obtained from different subjects. Information about the biological specimens (such as clinical information about the subject) are correlated with the results of analyzing the images, to obtain relationships between the information and the reaction. For example, the stage of a tumor can be correlated with the presence of a particular biomarker, such as an immunohistochemical (IHC) marker, or gene amplification. The same gene of interest (such as HER-2) can be analyzed at both DNA, RNA and protein level from different samples (or the same sample, with multi-color detection methods) and the results of these molecular analyses correlated with one another. This method is capable of efficiently obtaining many data points, because multiple tests can relatively quickly be performed on multiple similar copies of samples from multiple specimens. For example, if samples from at least 10 different tissue specimens are present in each of at least 10 different sections, and the ten different sections are respectively exposed to 10 different reagents, then 100 data points can quickly be obtained.

The power of this approach is even more evident if one sample is taken from each of 100 different tissue specimens and placed in a three dimensional matrix that is sectioned, into 300 sections. There would be 30,000 individual samples in the 300 sections that can be exposed to a variety of biological reagents to detect biomarkers. If 300 samples were taken from each of the 100 different tissue specimens, and placed in 300 different three dimensional matrices that were subsequently sectioned into 300 sections, three million distinct samples would be present in the 90,000 sections that would result. Exposing the 90,000 different sections to many different reagents (such as different probes) rapidly provides a large number of data points from which biological conclusions can be drawn with statistical confidence. Reactions with the biological reagents can also be correlated with clinical information associated with the tissue specimens.

This large scale arraying system can array specimens from a large number of specimens, or a large number of samples from one or more specimens can be arrayed. For example, a multi-tumor array could include hundreds or thousands (for example 5000 or more) different tumors, representing many (for example 135 or more) different types of tumors, and examples of corresponding normal tissue (e.g. 34 different normal tissues of the same type from which the tumors developed). Such an array can provide a template for a systematic and comprehensive analysis of disease genes, molecular alterations, etc. in substantially an entire spectrum of human neoplastic disease. Alternatively, an array of different breast cancer tumors could be made and distributed for molecular and other analyses at different locations, for example throughout a country or even globally.

In a particular embodiment of the method, the specimens are embedded in embedding medium to form tissue donor blocks, which are stored at identifiable locations in a donor array. The donor blocks are retrieved from the donor array, coordinates of particular areas in each of the tissue specimens in the donor blocks are determined, and tissue samples from the donor blocks (such as elongated punches) are retrieved and inserted into receptacles of corresponding size (such as punched holes) in different recipient tissue microarray blocks. After repeating this process with multiple donor blocks, to form a three-dimensional array of substantially parallel elongated samples from a variety of different specimens, the recipient tissue microarray blocks are then sectioned to make multiple similar tissue microarray sections that include samples of many different specimens. Each of these sections can then be subjected to treatment with multiple reagents, and subsequently analyzed for the presence of biological markers. This analysis can be performed by obtaining digital images of each section, or the samples in each specimen, and processing the image to identify specific regions of the section or sample that correspond to the presence of a biological marker, or to determine the amount and distribution of a biological marker that is present in the tissue microarray section. This information can be stored in a database for subsequent analysis and correlation with other information about the specimens and samples (such as clinical stage,, or co-alteration of gene copies or expression).

In yet another iteration, the invention is a computer implemented system for rapid construction and analysis of tissue microarray sections, in which a recipient block retriever obtains recipient blocks from a recipient block array, and transfers recipient blocks to a sectioner, which cuts sections from the recipient blocks, and mounts the sections on a solid support. A conveyor transfers the mounted sections to a processor, which processes the samples for biological analysis. An image analyzer obtains images of the tissue microarray sections, and either provides these to a pathologist to interpret or performs quantitative analysis for the presence of biological structures of interest, such as biological markers. A database stores information identifing tissue samples which are analyzed, and also stores information obtained from analysis of tissue microarray sections for correlation with other information available on these cases The computer implemented system can include a plurality of different stations for the sectioner, processor and image analyzer, a conveyor that transports mounted samples between stations, a plurality of robotic arms that expose the mounted sections to biological reagents for biological analysis, and a controller directing the transport of mounted sections to stations, the time that samples remain at individual stations, and the amount of time that sections are exposed to biological reagents.

The multiple recipient blocks can be constructed with corresponding samples at corresponding positions in the array (for example, at the same X-Y or other coordinate positions) because this arrangement facilitates tracking and identification of samples (and the specimens from which they come) in the different recipient blocks. However, the location of the sample in each block can also be randomized, and the sample (and the specimen from which it came) can be tracked, for example by a computer implemented system that associates the location of each sample in the array with a tissue specimen from which the sample was obtained. Alternatively, multiple (for example five or more) samples could be taken from each biological specimen, and placed in random locations in each recipient block. These multiple corresponding specimens could serve as an internal control on the accuracy of the analyzer (either human or automated), because similar results would be expected from the randomly located samples. The array constructor could also include a “scrambling” function in which the arrays are purposefully made with tissue specimens in non-corresponding locations, so that a manual interpreter of the results would not be influenced by the expectation that identical samples will be present at identical locations. Conversely, similar kind of samples from multiple tissues can be placed next to one another for simple visualization of the results at the microscope. Alternatively, multiple samples (e.g. normal and paired tumor tissues) from a given block can be arrayed next to one another in the resulting tissue microarray. The computerized system can keep track of the specimen locations in each array, even if their positions are randomly scrambled. It can then display the data in any order the observer wishes. Different permutations of this and other aspects of the present technology are quite varied.

Moreover, although certain aspects of the disclosed method and device are disclosed as being automated (such as microarray fabrication, sectioning, reagent processing, and image acquisition and analysis), any of these steps can be performed manually, or in other than an automated fashion as described in WO 99/44062 and WO 99/44063, herein incorporated by reference. Certain of these aspects may be disclosed as automated, and may be used in combination with other of these aspects that are not automated. For example, array construction may be automated, while sectioning and subsequent steps may not be automated. Alternatively, sectioning may be automated, while examination and interpretation of the sections may be performed manually.

The present disclosure also provides an approach for presenting multiple tissue samples to an examiner, in a manner that facilitates review of the samples and can improve uniformity of standards of examination of biological materials, such as histologic or molecular diagnostic examination of tissue specimens. The biological samples are presented in an array, in which the biological materials are at assigned positions which correspond to identifying information about the sample. The arrays can be prepared at a single location, to help avoid differences in procedures for preparing the biological material that can affect subsequent interpretation of results and tissue diagnosis. All of the biological materials can be simultaneously subjected to diagnostic or other techniques (such as exposure to histologic stains and molecular markers) that will also diminish differences that can produce discordant results. In particular embodiments, multiple substantial copies of each of the arrays is provided, for distribution to multiple recipients. The multiple substantial copies can be provided either by sectioning a substrate into which the biological samples have been placed, and/or by photographic or digital duplication and transmission.

The array can provide a relatively fast and convenient approach for examination of a large number of tissue specimens under substantially identical conditions by one or more persons, who can provide a much more uniform interpretation of results than is possible with multiple examiners at multiple locations. The resulting arrays also provide an important teaching tool that can be used by trainers and trainees to more conveniently display and examine large numbers of biological specimens under a microscope. Diagnostic interpretation of the samples in the array can also be normalized, to provide a standard set or guidelines for the interpretation of a given staining pattern that then can be used as a more uniform instruction to trainees and for the quality control of clinical assays.

In one embodiment of the method, a plurality of biological samples are provided at identifiable positions in the array, and the samples are subjected to a biological analysis. The biological analysis is usually performed after the samples are placed in the array, although the analysis can be performed prior to placement of the sample in the array. The array is then examined to detect a biological, histological or clinical marker, such as (a) the presence of a histologic sign of disease (e.g. cellular atypia or pyknotic nuclei) or (b) the presence of a molecular marker (such as an immunohistochemical marker or a nucleic acid probe) which is specifically bound to a substrate in the biological sample. The biological samples in the array may be samples of different tissue specimens (such as samples from many different tumors), or multiple samples from a single tissue specimen (for example to assess tissue homogeneity or heterogeneity). Alternatively, the biological samples in the array can include samples from different tissue specimens, as well as multiple samples from a single tissue specimen (for example, multiple copies of normal tissue as an internal control). This allows standardization of the molecular results from different sections of the same array or between multiple tissue microarray blocks that have different samples, but the same references included. The multiple substantial copies of the array can be subjected to the same biological analysis (such as immunohistochemical staining or molecular probing), or to different biological analyses, for example at a single location or at multiple different locations. The biological analysis may be performed, for example with a specific binding agent, such as an antibody or a nucleic acid probe, which substantially only or specifically recognizes and binds to a biological substrate of interest.

The multiple sections obtained from multiple tissue samples may vary slightly from one another. This variability may be due to the fact that the tissue morphology varies slightly from one location to another, or from the fact that the morphology changes as one cuts sections through the tissue microarray block. This variability can be controlled, for example by only including in the analysis donor blocks that have sufficient quantities of representative tumor areas, and blocks that have sufficient “depth” with representative tissue material. In addition, one would not need to include in array construction a particular case after the useful tissue area is used up. Variation in section morphology can be controlled by evaluating the morphology of the sections after a morphological stain, such as hematoxylin-eosin staining. This will enable the observer to determine which sections are likely to be representative.

In one embodiment, one can study the degree of intra-tumor heterogeneity of a biomolecule by acquiring a plurality of sections (for example, about 10, about 100, about 1000, or about 10,000 sections) from a given set of tumors, and testing a staining for the biomolecule in any number of these sections, such as at regular intervals (for example, about every 5th, 10th, 50^{th}, 100^{th} or 500^{th} section) from each tissue microarray block constructed.

In one embodiment, the multiple substantial copies of the array are obtained by providing elongated samples, substantially parallel to one another, at identifiable locations in a substrate, and sectioning the substrate. At least one of the multiple substantial copies may be subjected to a reference biological analysis, and multiple substantial copies are disseminated to one or more others to subject the copies to the same biological analysis, and compare the results of the same biological analysis to the reference biological analysis. This embodiment allows purchasers of test kits (such as kits containing IHC or nucleic acid probes) to perform an analysis and compare their results to a standard. If the purchaser obtains a different result, then modifications can, be made in the purchaser's techniques until the purchaser's result conforms to the result shown in the standard.

Alternatively, the substantial copies (array sections) can be disseminated to different researchers who can all perform the same or different biological analyses on the uniformly prepared tissue, and who can compare the results of their biological analyses to the reference biological analysis.

The substantial copies (e.g. different sections) of the array can be used for a broad variety of additional purposes. When the array is used for quality control purposes, an interpretation of the same biological analysis performed by different researchers can be compared to a reference interpretation of the reference biological analysis. For example, the comparison can determine whether a reagent used by the different researchers performs comparably to a reagent used in the reference biological analysis. When the array is used for training purposes (for example with medical students or pathology residents), the trainees can indicate a proposed interpretation of the biological analysis, and the proposed interpretation is compared to a reference interpretation of the reference biological analysis. In some embodiments, the trainees are test takers, who are graded by comparing the proposed interpretation to the reference interpretation. The reference interpretation need not be the interpretation of a single individual, but can instead be obtained by combining an interpretation of multiple referees. The trainess can also evaluate images, not the actual sections. The trainee can also be a computer controlled program/imaging system that is calibrated to give the same interpretation from a given set of tissue microarrays as a panel of experts.

The array which has been subjected to the biological analysis may be disseminated to multiple viewers at multiple locations, for example in electronic form, such as through a communication channel or a computer readable medium. The communication channel may be a global communication system, such as the INTERNET (for example as an attachment to an e-mail), and the computer readable medium may be a CD-ROM, DVD-ROM, or any other optical, magnetic or other data storage medium.

In particular disclosed embodiments, the array may be a microarray, for example in which the plurality of biological samples includes at least 100, 500 or even 1000 or more biological samples placed at identifiable positions in the microarray. The identifiable positions may be coordinates of the array, such as coordinates of a substantially uniform matrix of rows and columns. Identifiers (such as electronic identifiers) can be associated with the array, and diagnoses may be associated with the identifiers. In this manner, a viewer may conveniently immediately determine an interpretation associated with a sample, for immediate confirmation of a correct interpretation or correction of an incorrect interpretation.

The array is particularly suitable for displaying tissue specimens, such as pathology specimens. In some examples, the pathology specimens are neoplastic tissue, non-neoplastic tissue, a combination of neoplastic and non-neoplastic tissue, and/or comparative specimens of different examples in a biological spectrum. For example, the comparative specimens may be different stages in development of a tumor, different types of tumor; and/or different stages in progression of a biologically dynamic tissue (such as uterine endometrial tissue at different days during a menstrual cycle). The samples may also include multiple different types of histological and biological regions of interest from a given tissue or tumor, defined by a user.

This disclosure also concerns a method of examining biological samples by placing a plurality of elongated biological samples at identifiable positions in a substrate that is capable of being sectioned, sectioning the substrate to provide a plurality of substantial copies of an array of the biological samples, with the samples at the identifiable positions, identifying one or more reference copies, disseminating one or more dissemination copies to others, and comparing a biological interpretation of one or more dissemination copies to a biological interpretation of one or more reference copies. The reference copies may, for example, be included with a test kit.

The use of such multiple specimens allows one to examine the variability in assaying a particular biomolecule from tissue sections, as well as to continue and minimize such variability. The biological interpretations of one or more dissemination copies may be combined to provide a composite reference copy interpretation (such as testing the variability of tumor grading or stain evaluation by different pathologists and averaging of the grades of a tumor as assigned by an expert panel of pathologists). The biological samples can also be used as a convenient holder for a library of multiple tissue samples, to replace space consuming libraries of slides on which tissue sections are mounted. Information about subjects from whom the samples were obtained can also be associated with each sample, and readily retrieved (for example electronically) so that clinical information (including clinical course) can be linked to the tissue.

In yet other embodiments, the multiple sections (or other copies) are disseminated to different recipients, who indicate an interpretation of the samples in the array, and communicate the interpretation to different recipients or a central source. In this manner, a pooled interpretation of the samples may be obtained from a small or large group of experts. Alternatively, the multiple interpretations thus obtained could be used to determine the extent of variability in interpretation of a particular tumor, disease, or pathologic/histological feature.

The array technology described in this disclosure is versatile, and allows a variety of different biological samples (for example samples from at least 10 different tissue specimens present in each different section) to be exposed to a variety of different biological analyses (for example at least 10 different reagents). Alternatively, the biological samples are obtained from at least 100 different tissue specimens, and are exposed to at least 100 different reagents. Images (such as digital images) of the arrays can be obtained, and the images analyzed, for example by quantifying the reaction with the substrate. The results of the biological analyses can be used for a variety of purposes, such as validating the presence of a particular biomerker in a set of tissues, determining the frequency and clinical associations of such a marker, evaluating a reagent for disease diagnosis or treatment; identifying a prognostic marker for cancer; assessing or selecting therapy for a subject; and/or finding a biochemical target for medical therapy. The biological sample may be a tissue specimen, as well as a hematological or cytological preparation of cells.

Explanations of Terms

An “annotation”, when used in the context of a region of interest, a tissue sample, a tissue specimen, a tissue section, a tissue microarray, a tissue donor block, or a recipient block, refers to retrievably stored information that relates to the region of interest, the tissue sample, the tissue specimen, the tissue section, the tissue microarray, the tissue donor block, or the recipient block. For example, an annotation may be retrievably stored information regarding the source, of a tissue sample; clinical, medical or demographic information about the donor of the tissue specimen; time, manner, location and/or institution in which the specimen was obtained; method of fixation, if any; type of tissue; histological or pathological features observable within the tissue, such as tumor type, tumor grade, acute and/or chronic inflammation, thromboses, or examples of normal (nondiseased) tissue or cells; information that enables location of histological or pathological features; tissue, cellular, or subcellular location and/or quantity of biological markers of interest; location information regarding one or more reference points or indicia in a tissue section, a tissue microarray section, or a tissue donor block; information regarding the distance of one or more reference points or indicia from a region of interest; information that enables location and/or retrieval of other tissue samples, tissue specimens, tissue sections, tissue microarrays, tissue donor blocks, or recipient blocks, that may share one or more features in common with the tissue sample, section, microarray, etc. that is the subject of the annotation. The descriptions of the types of annotations that are possible is intended to be illustrative and not exhaustive. Virtually any type of information may the subject of an annotation. For example, an investigator may hypothesize that the development of a particular type of cancer, or a particular inflammatory or infectious disease, is related to an individual's family history, astrological sign, birthplace, level of education, or exposure to a particular kind of animal. All such information could readily be stored as an annotation associated with the region of interest, tissue sample, tissue section, tissue microarray, tissue donor block, or recipient block. The annotation would then be available for review, and could serve as a tag for locating and/or retrieving tissue specimens, tissue microarrays, regions of interest, etc.

An “array” refers to a grouping or an arrangement, without necessarily being a regular arrangement.

A “biological analysis” or “bioanalysis” is an analytical technique for obtaining biological information about a substrate, such as a tissue specimen. Particular example of such techniques are the use of histological stains (such as H&E), immunohistochemical markers such as labeled antibodies for antigens of interest, and nucleic Acid probes for detecting mRNA, DNA and other nucleic acids in the cells. Antibodies and other genetically engineered detection probes, antibodies and reagents can be used. Nucleic acid probes could be used on proteins and antibodies to detect nucleic acid targets.

A “biological marker” is a biomolecule, a biochemical label, or other biological label that identifies a structure or function of interest in a biological specimen.

A “biological substrate of interest” is one or more biological markers which are being observed by an observer.

A “biomolecule” is any molecule which is synthesized in any living cell, or used by living cells in biosynthetic pathways. The term includes, for example, nucleic acids, proteins, carbohydrates, lipids and lipid derivatives, amino acids, nucleotides, nucleosides, prostaglandins, and the like. Additional examples may be found in Stryer, Biochemistry, 4th ed. 1995.

“Cell free analysis” is a subset of biological analysis, in which the biological substrate of interest is partially or completely isolated from a cell prior to observing the biological substrate of interest. The biological substrate of interest may be any biomolecule, or a plurality of biomolecules. Examples of cell free analysis are innumerable, and include DNA sequencing, restriction fragment length polymorphism determination, Southern blotting and other forms of DNA hybridization analysis, determination of single-strand conformational polymorphisms (Sakar et al., Nucleic Acid Res 1992; 20:871-8), comparative genomic hybidization (Kallioniemi et al., Science. 258: 818-21, 1992), mobility-shift DNA binding assays, protein gel electrophoresis, Northern blotting and other forms of RNA hybridization analysis, protein purification, chromatography, immunoprecipitation, protein sequence determination, Western blotting (protein immunoblotting), ELISA and other forms of anybody-based protein detection, isolation of biomolecules for use as antigens to produce antibodies, PCR, RT PCR, differential display of mRNA by PCR (known in the art as differential display; Liang et al., Science 1992;257:967-72), serial analysis of gene expression (U.S. Pat. No. 5,695,537), protein truncation test (Wimmer et al., Human Mutation. 16(1):90-1, 2000; Moore et al., Molecular Biotechnology. 14(2):89-97, 2000; Den Dunnen et al., Human Mutation. 14(2):95-102, 1999). Protocols for carrying out these and other forms of cell free analysis are readily available to those skilled in the art, for example in Ausubel et al., Current Protocols in Molecular Biology, (c) 1998, John Wiley & Sons Ausubel et al., Short Protocols in Molecular Biology, (c) 1999, John Wiley & Sons; Maniatis et al., Molecular Cloning: A Laboratory Manual; and the series of publications known as Methods in Emzymology.

A “communication channel” or “network” is a system, such as the internet, which permits digital dissemination of digital information, such as digital images and test associated with the images. An example of such a communication channel is shown in PCT publication WO 99/30264, which discloses a digital telepathology imaging system, and is incorporated by reference.

A “copy” of a section refers to substantial similarity, and not absolute identity.

A “donor block” can include a substrate into which has been introduced solid donor tissue or a cell suspension, or any other biological tissue.

By “polypeptide” is meant any chain of amino acids, regardless of length or post-translation modification (e.g., glycosylation or phosphorylation).

A “gene amplification” is an increase in the copy number of a gene, as compared to the copy number in normal tissue. An example of a genomic amplification is an increase in the copy number of an oncogene. A “gene deletion” is a deletion of one or more nucleic acids normally present in a gene sequence, and in extreme examples can include deletions of entire genes or even portions of chromosomes. Gene amplifications and deletions are examples of variations in gene copy number.

A “genomic target sequence” is a sequence of nucleotides located in a particular region in the human genome that corresponds to one or more specific loci, gene, or specific DNA sequence, including genetic abnormalities, such as a nucleotide polymorphism, a deletion, or an amplification.

A “genetic disorder” is any illness, disease, or abnormal physical or mental condition that is caused or suspected to be caused by an alteration in one or more genes or regulatory sequences (such as a mutation, deletion or translocation).

“Immunohistochemical” (abbreviated IHC) refers to specific binding agents, such as polyclonal and monoclonal antibodies, which recognize and mark antigens of interest, often by a chemical that shows that the agent has bound to the antigen of interest. An example of an IHC agent is HER-2 monoclonal antibody.

A “nucleic acid array” refers to an arrangement of nucleic acids (such as DNA or RNA) in assigned locations in the arrangement, such as that found in cDNA or CGH arrays.

A “microarray” is an array that is miniaturized so as to require microscopic examination for visual evaluation.

A “DNA chip” is a DNA array in which multiple DNA molecules (such as cDNAs) of known DNA sequences are arrayed on a substrate, usually in a microarray, so that the DNA molecules can hybridize with nucleic acids (such as cDNA or RNA) from a specimen of interest. DNA chips are further described in Ramsay, Nature Biotechnology 16: 40-44, 1998, which is incorporated by reference.

Unless indicated otherwise by context, a “tissue specimen” refers to an intact piece of tissue, for example embedded in medium. A “tissue sample” refers to a sample taken from the specimen, or a sectioned portion of the sample. A sample can be either a tissue sample or a sample of other biological material, such as a liquid cellular suspension.

“Comparative Genomic Hybridization” or “CGH” is a technique of differential labeling of test DNA and normal reference DNA, which are hybridized simultaneously to chromosome spreads, as described in Kallioniemi et al., Science 258:818-821, 1992, which is incorporated by reference.

“Gene expression microarrays” refers to microscopic arrays of cDNAs printed on a substrate, which serve as a high density hybridization target for mRNA probes, as in Schena, BioEssays 18:427-431, 1996, which is incorporated by reference.

“Serial Analysis of Gene Expression” or “SAGE” refers to the use of short sequence tags to allow the quantitative and simultaneous analysis of a large number of transcripts in tissue, as described in Velculescu et al., Science 270:484-487, 1995, which is incorporated by reference.

“High throughput genomics” refers to application of genomic or genetic data or analysis techniques that use microarrays or other genomic technologies to rapidly identify large numbers of genes or proteins, or distinguish their structure, expression or function from normal or abnormal cells or tissues.

An observer can be a person viewing a slide with a microscope or an observer who views digital images acquired. Alternatively, an observer can be a computer-based image analysis system, which automatically observes, analyses and quantitates biological arrayed samples with or without user interaction.

A “specific binding agent” is an agent that recognizes and binds substantially preferentially to a biological marker of interest, so that the agent provides potentially useful information about the biological marker. Examples of specific binding agents are polyclonal and monoclonal antibodies for an antigen of interest; proteins and proteins derivatives that interact or bind to to other (for example, calmodulin or a labeled calmodulin derivative;), and nucleic acid probes such as DNA and RNA probes.

The term “tissue” as used herein includes cellular specimens unless the context clearly dictates otherwise. Such cellular specimens include, for example, cervical cell samples, bronchial washings, cell samples obtained by endoscopy, blood cells, bacteria, fungi, yeasts, and the like.

A “tumor” is a neoplasm that may be either malignant or non-malignant. “Tumors of the same tissue type” refers to primary tumors originating in a particular organ (such as breast, prostate, bladder or lung). Tumors of the same tissue type may be divided into tumors of different sub-types (a classic example being bronchogenic carcinomas (lung tumors) which can be an adenocarcinoma, small cell, squamous cell, or large cell tumor).

The singular forms “a” or “an” or “the” include plural referents unless the context clearly dictates otherwise. Thus, for example, reference to “a section” includes a plurality of such sections, and reference to “a biological marker” includes reference to one or more biological marker and equivalents thereof known to those skilled in the art, and so forth.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the present specification, including definitions, will control. In addition, the materials, methods, and examples are illustrative only and are not intended to be limiting.

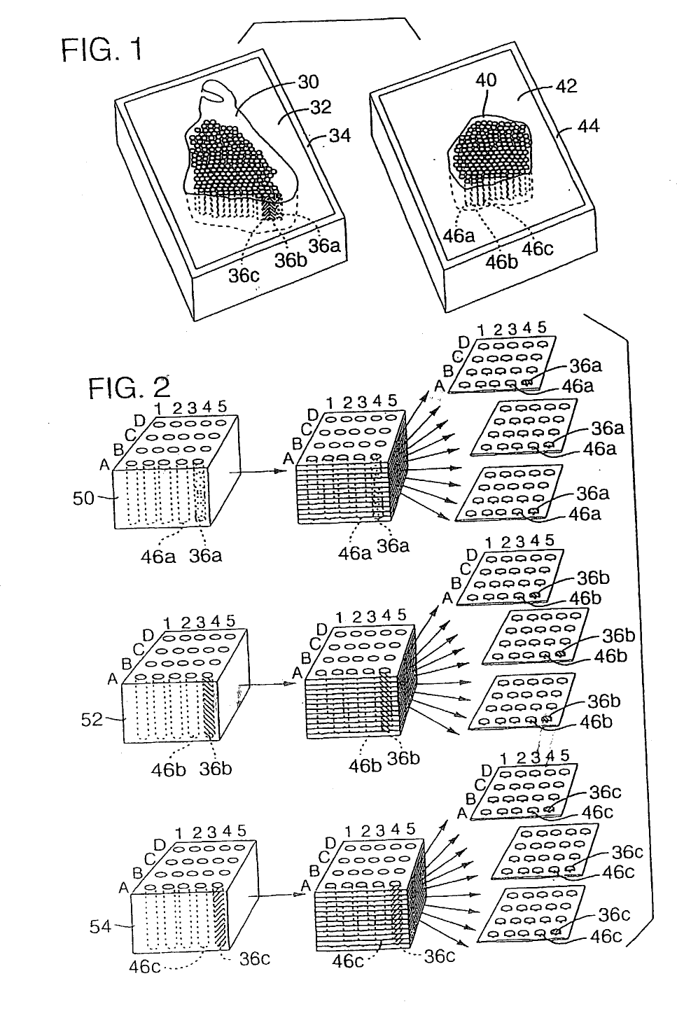

Overview of Method (FIGS. 1-4)

The present disclosure concerns an automated method and device for manufacturing large numbers of arrays of biological materials, such as tissue specimens, which can be subjected to rapid parallel analysis with a variety of different biological reagents, such as nucleic acid probes and antibodies. This high speed parallel analysis of samples from multiple sources (such as a variety of tumors) permits simultaneous detection of multiple biomarkers in the samples, and further allows correlations to be made about the presence, distribution (between and within tissues, between and within cells in a tissue) and quantity of biomarkers in different samples from different tissue specimens. This enables one to determine the precise population frequency of a biomolecule in a large population sample of a certain type of tissue, and quantitate precise frequencies of molecular alterations, for example molecular alterations of clinical, pathological, or disease-producing significance. This information can further be associated with clinical and demographic information about the subject from whom the tissue was taken (such as tumor stage) or other histological information (such as degrees of cellular atypia in the specimen) to obtain correlations (with high statistical significance). This approach is more fully disclosed in U.S. Provisional Application Nos. 60/106,038 and 60/075,979, and PCT publications WO 9944063A2 and WO 9944062A1, all of which are incorporated by reference in their entirety.

An overview of the production of microarrays for high throughput parallel and serial biological analysis of large numbers of biological samples, such as samples of tissue specimens, is shown in FIGS. 1-3. Parallel refers to the fact that multiple tissues can by analyzed at once from the same set of tissue specimens. The word “serial” refers to the fact that one can construct literally tens of thousands of tissue microarray sections, and these can be molecularly profiled one at a time to achieve a serial molecular profiling of many biomolecules in each of the tissue specimens on the array. The methods described here for the high-throughput automated arraying increase the speed and efficiency of both the parallel and serial “dimensions” of the tissue microarraying applications. The method also applies to nonautomated tissue microarraying applications.

A tissue specimen 30 is shown in FIG. 1 embedded in a block of embedding medium 32, which is carried by a container 34. Multiple punches of small diameter cylindrical sample cores 36 of material (for example 0.6 mm in diameter) are taken from specimen 30 (as illustrated by the small cylindrical openings in specimen 30). These specimens can come from histologically similar or identical regions of the tumor considered interesting or representative, or from histologically or biologically different regions within each tissue. Although hundreds of similar cylindrical cores of tissue specimen 30 are removed, for purposes of explanation three such sample cores 36a, 36b and 36c will be discussed. Each of these sample cores 36a, 36b and 36c is differently shaded to help trace them through the method illustrated in FIG. 2. The sample cores could be of any shape or configuration, but are shown as cylinders for ease of illustration.

For purposes of illustration, FIG. 1 also shows a second tissue specimen 40 embedded in embedding medium 42, which is carried by a container 44. Hundreds of small diameter cylindrical sample cores 46 are also taken from specimen 40, although for purposes of illustration only three such sample cores 46a, 46b and 46c are labeled. In addition to tissue specimens 30 and 40, hundreds or even thousands of different embedded tissue specimens may be available, and one sample or hundreds of cylindrical sample cores are obtained from each of those specimens. However, for purposes of this example, twenty different tissue specimens will be described as available as source material for the array, two of which are illustrated in FIG. 1.

FIG. 2 illustrates three substantially identical different receptacle blocks 50, 52, 54, each of which is made of paraffin or other suitable material, in which twenty cylindrical receptacles have been formed that are complementary in size and shape to the cylindrical sample cores punched or bored from the tissue specimen shown in FIG. 1. The cylindrical receptacles are substantially parallel and form an array in the block, and the array is labeled in FIG. 2 by coordinate positions A, B, C and D along one edge of the block, and 1, 2, 3, 4 and 5 along a perpendicular edge of the block. Hence each of the twenty receptacles in each block 50, 52 and 54 can be uniquely identified as receptacle A5, D1, etc.

Each of the cylindrical cores taken from the tissue specimen is placed in a corresponding position in the different blocks 50, 52 and 54, so that corresponding positions of the array can be more easily identified as corresponding to tissue samples from the same specimen. Hence sample cores 36a, 36b and 36c (all of which were sampled from tissue specimen 30 in FIG. 1) are inserted in the receptacle array at position A5 in blocks 50, 52 and 54. Similarly, sample cores 46a, 46b and 46c (all of which are sampled from tissue specimen 40 in FIG. 1) are inserted in the receptacle array at position A4 in blocks 50, 52 and 54. This process is repeated until sample cores are taken from twenty different tumors (not shown in FIG. 1) and placed in corresponding positions of the blocks to form a recipient array of parallel cores in each of the multiple receptacle blocks. Although only three receptacle blocks 50, 52 and 54 are shown in FIG. 2, as many blocks can be used as there are sample cores taken from each of the tissue specimens (which is often hundreds of sample cores).

Once the recipient arrays have been formed in the blocks 50, 52 and 54, the blocks are sectioned (with a sectioner, for example with a microtome). The block section cuts can be placed in many different orientations, but for purposes of illustration they are shown substantially transverse (at a right angle) to the longitudinal axes of the sample cores. The thickness of the block sections can be very small, for example 0.01 mm, so that 300 block sections would be obtained from a sample core that is 3 mm long and 0.6 mm in diameter. Each of the block sections is a substantial copy of the other sections in the array, and the tissue samples at each location in the array are from the same tissue specimen, and generally share common biological characteristics (such as gene or protein expression) that can be ascertained with biomarkers.

For example, block 50 is sectioned into 300 multiple block sections (only three of which are separately shown in FIG. 2) with specimen core 36a at position A5. After the block is sectioned, each of the sections retains sample 36a at position A5 (as shown by the dark color of 36a in all the views of block 50). Similarly, each of the sections of block 52 retains sample 36b at position A5, and each of the sections of block 54 retains sample 36c at position A5. Hence the corresponding positions A5 in the multiple sections will likely share biological characteristics that can be simultaneously analyzed by exposing the multiple different sections to different biological analyses.

Similarly, after the block is sectioned, each of the sections retains sample 46a at position A4. Each of the sections of block 52 retains sample 46b at position A4, and each of the sections of block 54 retains sample 46c at position A4. Hence the corresponding positions A4 in the multiple sections will likely share biological characteristics that can be simultaneously analyzed when the multiple different sections are exposed to the different biological analyses.

The results of the biological analyses for the samples at positions A1, A2, A3 . . . D5 can then be recorded, and the results will then allow one to establish the prevalence, distribution and quantity of a biomarker in the set of specimens analyzed. The results of biological analyses canalso be associated with clinical or other information that has been collected about each of the specimens. Moreover, biological patterns can be detected from the, large number of data points that can be quickly obtained by this method. For example, the same tumor is sampled from multiple sites, the degree of heterogeneity in biomarker expression can be directly ascertained from the tissue microarray analysis. Similarly, variations in gene copy numbers can be detected not only within, but also between the tissue samples, and independent characteristics about these samples can then be reviewed to determine whether variations in the gene copy number can be correlated with clinical or other information that is available about the sample.

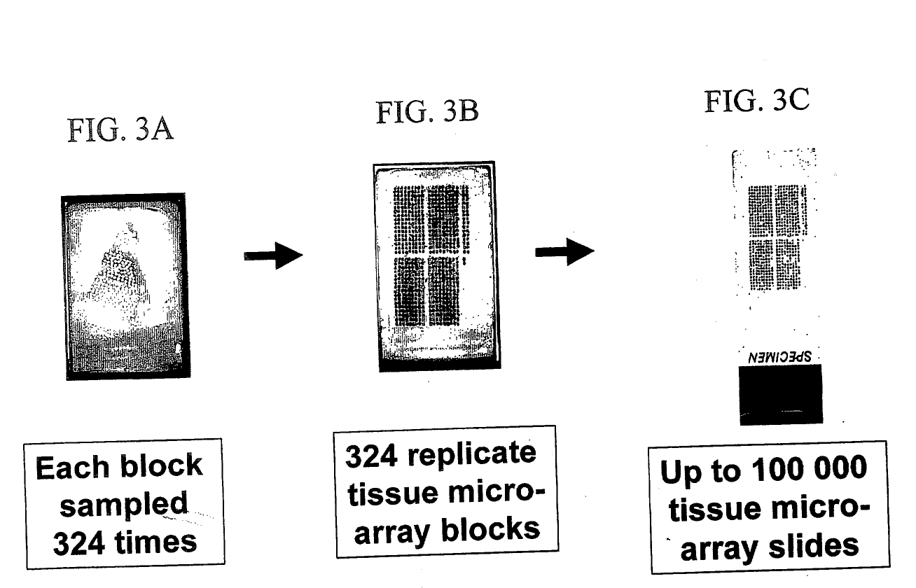

FIG. 3 helps illustrate this concept, by showing in FIG. 3A that 1000 different embedded tissue specimens can each be sampled 324 times (as shown by the 324 small holes in the top tissue specimen). Each of the 324 samples can be placed in 324 different recipient blocks (FIG. 3B). Each of the 324 recipient blocks has 1000 different samples arrayed in the block, each of the 1000 different samples having been obtained from each of the 1000 different tissue specimens. Once each of the 324 recipient blocks is sectioned into 300 sections, 97,200 tissue microarray slides (FIG. 3) are obtained, with each position in the array containing a sample from the same tissue specimen. If each of the 97,200 slides is then subjected to a different biological analysis (such as exposure to a DNA probe) then each of the 1000 different tissue specimens can undergo 97,200 different analyses, and 97,200,000 different data points can be obtained in this example.

The use of 324 samples in this example assumes that the dimension of useful tumor area is 15×15 mm in the block, and that the center to center distance of the sample punches in the tumor is 0.8 mm. Using these dimensions in a theoretical calculation, approximately 100,000 slides could be obtained, which in theory would be sufficient to have approximately one different slide for each gene in the human genome. However, a larger or smaller number of arrays could be made, depending on the size of the useful tumor area, the number of available tissue blocks per subject, and a depth of the original blocks. Although the sample morphology on all of the 100,000 slides will not be identical, the variability within one specimen could be compensated by similar variability in other locations. That is, one could obtain a representative sample of the population of tumors.

Uniformity of tissue morphology in a tissue specimen can also be a factor in determining the number of sample punches taken from a tissue specimen, because substantial changes in the coordinates (for example in X, Y or Z directions) could limit the area from which similar punches could be taken. However, the arrays can also be used for the purpose of taking samples from different areas of the specimen, for example from different tumor containing areas of the specimen. The arrays can also be used to sample different areas having different morphologies, for example by defining multiple types of cells or tissues from each block, and arraying them separately. For example surrounding stroma could be sampled, apart from the tumor itself, or invasive and non-invasive tumor present in the same tissue specimen can be separately sampled, add placed in a single or multiple arrays. Each of the areas from which such tissues are taken can be marked separately in advance, and the automated array can keep track of the origin or designation that is assigned to each sample in the array. It will be recognized that these possibilities are only a few examples of the multiple possibilities and permutations that can be used in association with the array technology disclosed herein.

Alternatively, multiple samples (for example 10-20 samples) may be taken from different sites within each tumor, and biomarkers can be evaluated at each of these sites. The information obtained from such an analysis would provide a comprehensive analysis (such as a quantitative analysis) of the impact of tumor heterogeneity, and could correlate patterns of heterogeneity with prior or subsequent tumor behavior, patient survival, etc.

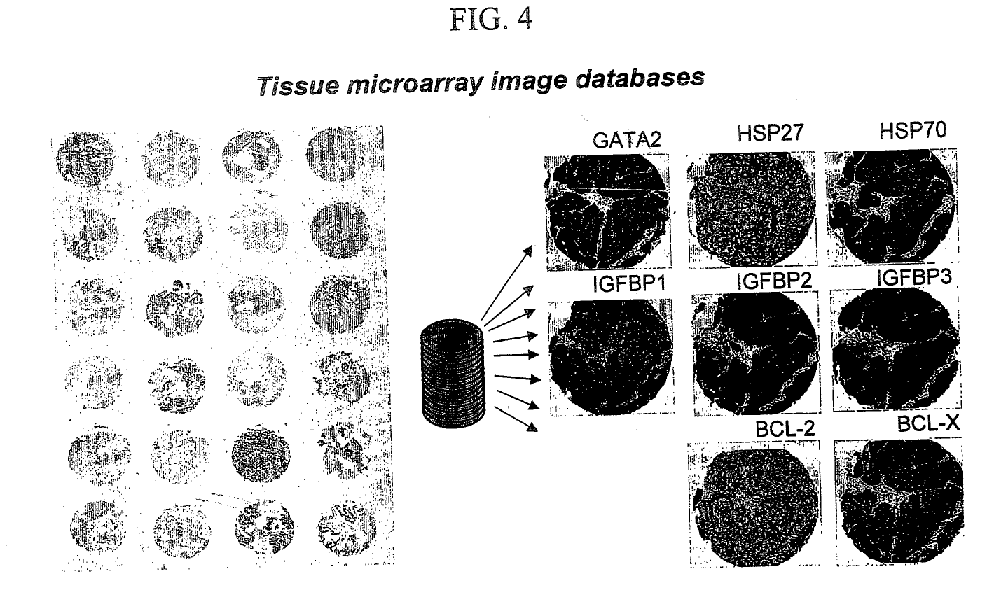

Image Analysis of the Tissue Microarray Experiments (FIG. 4)

FIG. 4 illustrates how the data points can be obtained by either parallel (left) or serial analysis (right) of tissue microarrays. Serial analysis is achieved by exposing different tissue microarray slides to different immunohistologic markers (although in alternative embodiments nucleic acid probes or many other reagents may be used to detect gene expressions or amplifications). The different microarray slides will contain different sections of a cylindrical tissue sample, which will then react with the nucleic acid probes or antibodies. The microarray slides can then be examined under a microscope, and changes in presence, quantity and distribution of a biomolecule can be accurately determined from each of the arrayed samples. For example, the frequency of a particular gene or gene mutation in a particular tissue type may be determined. The type of biomarker expression can e.g. be membranous, cytoplasmic, nuclear or combinations thereof. It can be uniformly or ununiformly distributed between and within cell types present in the tissues. The images of the samples (such as those shown in FIG. 4) can consist of both “horizontal” or “parallel” (left panel) (X-Y, i.e. different tissue sample spots that have been on the same slide and that have therefore been exposed to the same detection reagent). The parallel dimension allows one to determine the frequency, pattern and quantity of a biomolecule in tissue spots on the same tissue microarray slide. The other dimension is “vertical” or “serial” dimension (right panel) (Z-axis, i.e. the different sections of the same tissue, or multiple different parameters analyzed from the multiple section copies). In the example of a serial application, the same prostate cancer tissue microarray was cut into multiple consecutive sections that were each stained with a different reagent (in this case antibodies to eight gene products suspected to have an importance in prostate cancer). Out of these multiple tissue microarrays, the example here contains images of only a single tumor, which is now profiled with eight different antibodies.

Automated or manual image acquisition may be based on collecting images of an entire slide (such as the subsection of the array shown in FIG. 4) or from each spot separately. The latter approach will then enable one to form a database of images that can be displayed either in the original order and position (left panel), in a rearranged manner to display similar types of tumors next to one another, or by displaying multiple different staining results from the same sample (different sections of the same core sample). Based on the example of FIGS. 3A to 3B, one could therefore stain/hybridize up to 100,000 sections from a given set of tumors with different reagents, and acquire images of all the tissue spots. These images could be then rearranged for display in a number of different formats based on the general principles shown in FIG. 4.

Images of the experiments indicated above were acquired with a Zeiss Progress Camera connected to a Zeiss Axiophot camera with a manual XYZ stage. Multiple prostate cancer tissue microarray sections were each stained with a particular antibody according to the manufacturers' instructions. The staining is reflected as a brown immunoperoxidase precipitate. It can be readily distinguished that that the markers mostly had a membranous or cytoplasmic staining. Similarly, nuclear or membranous staining could be distinguished. An image analysis system or a manual observation of the images may now depict the type of staining, the variability of the staining within and between tissue spots, within and in between individual cells in the spots, determine the staining intensity qualitatively, semi-quantitatively (scale 0 to 4, for example) or in a full quantitative analysis of the staining reaction (gray scale values, for example from 1-256, or as a color/hue of the specimens). The imaging system could separately determine the staining reaction in different parts of the spot, such as separately in stromal or carcinoma components. A computer may be used to compare automatically the staining results between adjacent sections of the same tissue microarray with same or different antibodies to form ratios of molecular intensities. Multiple images of the same or different spots could be subjected to an automated multi-parametric analysis, where one could clasify tumors based on the intensity, distribution or other features of multiple stainings on consecutive tissue microarray sections.

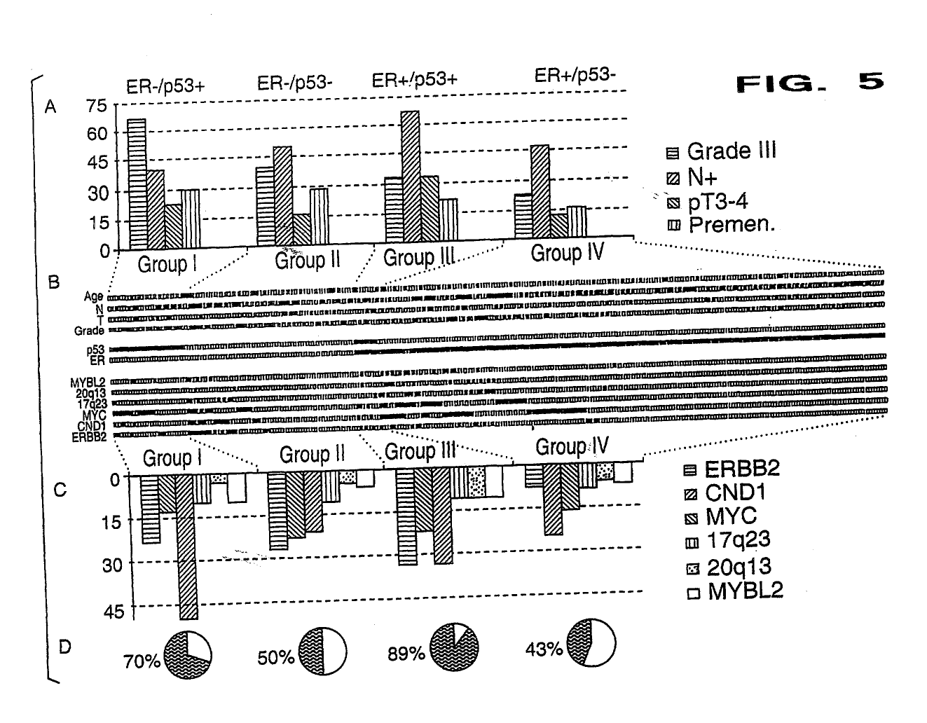

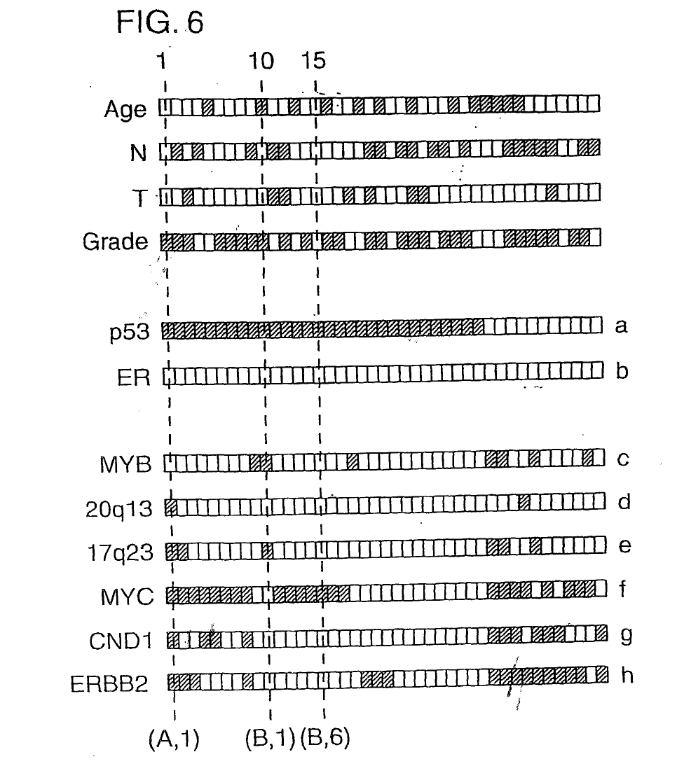

Overview of Data Correlation in FIGS. 5-6

The potential of the array technology of the present invention to perform rapid parallel molecular analysis of multiple tissue specimens is illustrated in FIGS. 5A-5D, where the y-axis of the graphs in FIGS. 5A and 5C corresponds to percentages of tumors in specific groups that have defined clinicopathological or molecular characteristics. This diagram shows correlations between clinical and histopathological characteristics of the tissue specimens in the micro-array. Each small box in the aligned rows of FIG. 5B represents a coordinate location in the array. Corresponding coordinates of consecutive thin sections of the recipient block are vertically aligned above one another in the horizontally extending rows. These results show that the tissue specimens could be classified into four classifications of tumors (FIG. 5A) based on the presence or absence of cell membrane estrogen receptor expression, and the presence or absence of the p53 mutation in the cellular DNA. In FIG. 5B, the presence of the p53 mutation is shown by a darkened box, while the presence of estrogen receptors is also shown by a darkened box. Categorization into each of four groups (ER−/p53+, ER−/p53−, ER+/p53+ and ER+/p53−) is shown by the dotted lines between FIGS. 5A and 5B, which divide the categories into Groups I, II, III and IV corresponding to the ER/p53 status.

FIG. 5B also shows clinical characteristics that were associated with the tissue at each respective coordinate of the array. A darkened box for Age indicates that the patient is premenopausal, a darkened box N indicates the presence of metastatic disease in the regional lymph nodes, a darkened box T indicates a stage 3 or 4 tumor which is more clinically advanced, and a darkened box for grade indicates a high grade (at least grade III) tumor, which is associated with increased malignancy. The correlation of ER/p53 status can be performed by comparing the top four lines of clinical indicator boxes (Age, N, T, Grade) with the middle two lines of boxes (ER/p53 status). The results of this cross correlation are shown in the bar graph of FIG. 5A, where it can be seen that ER−/p53+ (Group I) tumors tend to be of higher grade than the other tumors, and had a particularly high frequency of myc amplification, while ER+/p53+ (Group III) tumors were more likely to have positive nodes at the time of surgical resection. The ER−/p53− (Group II) showed that the most common gene amplified in that group was erbB2. ER−/p53− (Group II) and ER+/p53− (Group IV) tumors, in contrast, were shown to have fewer indicators of severe disease, thus suggesting a correlation between the absence of the p53 mutation and a better prognosis.

This method was also used to analyze the copy numbers of several other major breast cancer oncogenes in the 372 arrayed primary breast cancer specimens in consecutive FISH experiments, and those results were used to ascertain correlations between the ER/p53 classifications and the expression of these other oncogenes. These results were obtained by using probes for each of the separate oncogenes, in successive sections of the recipient block, and comparing the results at corresponding coordinates of the array. In FIG. 5B, a positive result for the amplification of the specific oncogene or marker (mybL2, 20q13, 17q23, myc, cnd1 and erbB2) is indicated by a darkened box. The erbB2 oncogene was amplified in 18% of the 372 arrayed specimens, myc in 25% and cyclin D1 (cnd1) in 24% of the tumors.

Two recently discovered novel regions of frequent DNA amplification in breast cancer, 17q23 and 20q13, were found to be amplified in 13% and 6% of the tumors, respectively. The oncogene mybL2 (which was recently localized to 20q13.1 and found to be overexpressed in breast cancer cell lines) was found to be amplified in 7% of the same set of tumors. MybL2 was amplified in tumors with normal copy number of the main 20q13 locus, indicating that it may define an independently selected region of amplification at 20q. Dotted lines between FIGS. 5B and 5C again divide the complex co-amplification patterns of these genes into Groups I-IV which correspond to ER−/p53+, ER−/p53−, ER+/p53+ and ER+/p53−.

FIGS. 5C and 5D show that 70% of the ER−/p53+ specimens were positive for one or more of these oncogenes, and that myc was the predominant oncogene amplified in this group. In contrast, only 43% of the specimens in the ER+/p53− group showed co-amplification of one of these oncogenes, and this information could in turn be correlated with the clinical parameters shown in FIG. 5A. Hence the microarray technology of the present invention permits a large number of tumor specimens to be conveniently and rapidly screened for these many characteristics, and analyzed for patterns of gene expression that may be related to the clinical presentation of the patient and the molecular evolution of the disease. In the absence of the microarray technology of the present invention, these correlations are more difficult to obtain.

A specific method of obtaining these correlations is illustrated in FIG. 6, which is an enlargement of the right hand portion of FIG. 5B. The microarray in this example is arranged in sections that contain seventeen rows and nine columns of circular locations that correspond to cross-sections of cylindrical tissue specimens from different tumors, wherein each location in the microarray can be represented by the coordinates (row, column). For example, the specimens in the first row of the first section have coordinate positions (A,1), (A,2) . . . (A,9), and the specimens in the second row have coordinate positions (B,1), (B,2) . . . (B,9). Each of these array coordinates can be used to locate tissue specimens from corresponding positions on sequential sections of the recipient block, to identify tissue specimens of the array that were cut from the same tissue cylinder.

FIG. 6 illustrates one conceptual approach to organizing and analyzing the array, in which the rectangular array may be converted into a linear representation in which each box of the linear representation corresponds to a coordinate position of the array. Each of the lines of boxes may be aligned so that each box that corresponds to an identical array coordinate position is located above other boxes from the same coordinate position. Hence the boxes connected by dotted line 1 correspond to the results that can be obtained by looking at the results at coordinate position (A,1) in successive thin sections of the donor block, or clinical data that may not have been obtained from the microarray, but which can be entered into the system to further identify tissue from a tumor that corresponds to that coordinate position. Similarly, the boxes connected by dotted line 10 correspond to the results that can be found at coordinate position (B,1) of the array, and the boxes connected by dotted line 15 correspond to the results at coordinate position (B,6) of the array. The letters a, b, c, d, e, f, g, and h correspond to successive sections of the donor block that are cut to form the array.

By comparing the aligned boxes along line 1 in FIG. 6, it can be seen that a tumor was obtained from a postmenopausal woman with no metastatic disease in her lymph nodes at the time of surgical resection, in which the tumor was less than stage 3, but in which the histology of the tumor was at least Grade III. A tissue block was taken from this tumor and is associated with the recipient array at coordinate position (A,1). This array position was sectioned into eight parallel sections (a, b, c, d, e, f, g, and h) each of which contained a representative section of the cylindrical array. Each of these sections was analyzed with a different probe specific for a particular molecular attribute: In section a, the results indicated that this tissue specimen was p53+; in section b that it was ER−; in section c that it did not show amplification of the mybL2 oncogene; in separate sections d, e, f, g and h that it was positive for the amplification of 20q13, 17q23, myc, cnd1 and erbB2.

Similar comparisons of molecular characteristics of the tumor specimen cylinder that was placed at coordinate position (B,1) can be made by following vertical line 10 in FIG. 6, which connects the tenth box in each line, and corresponds to the second row, first column (B,1) of the array. Similarly the characteristics of the sections of the tumor specimen cylinder at coordinate position (B,6) can be analyzed by following vertical line 15 down through the 15^{th} box of each row. In this manner, parallel information about the separate sections of the array can be performed for all positions of the array. This information can be presented visually for analysis as in FIG. 6, or entered into a database for analysis and correlation of different molecular characteristics (such as patterns of oncogene amplification, and the correspondence of those patterns of amplification to clinical presentation of the tumor).

In the particular examples above, the staining intensity of FISH result is condensed to the mere presence or absence of a biomarker. However, image analysis techniques, or even semi-quantitative manual scoring can be used to determine the staining intensity with a particular antibody. The same principle applies to quantitation of DNA copy number changes, mRNA in situ hybridization or other molecular analyses. Similarly, statistical analyses could be performed, or the data displayed in a quantitative manner, for example in gray scales, or colors.

Analysis of consecutive sections from the tumor arrays enables co-localization of hundreds of different DNA, RNA, protein or other targets in the same cell populations in morphologically defined regions of every tumor, which facilitates construction of a database of a large number of correlated genotypic or phenotypic characteristics of uncultured human tumors.

The fact that the same tissue can also be analyzed at at the gene, mRNA, or protein level, enables the determination of the level of the molecular alteration affecting a particular tissue or tumor. For example, a tumor may have DNA amplification, which leads to increased mRNA ad protein expression. Alternatively it is possible to observe elevation of mRNA expression only, without associated changes in protein level (for example as might occur due to different patterns of protein degradation). Knowledge of the relationships of gene, mRNA and protein will qualitatively and quantitatively enhance understanding tumor biology, development of diagnostics, and defining therapeutic targets. Such multiple determinations are made possible by tissue microarray technology.

Scoring of mRNA in situ hybridizations or protein immunohistochemical staining is also facilitated with tumor tissue microarrays, because hundreds of specimens can be analyzed in a single experiment. The tumor arrays also substantially reduce tissue consumption, reagent use, and workload when compared with processing individual conventional specimens one at a time for sectioning, staining and scoring. The combined analysis of several DNA, RNA and protein targets provides a powerful means for stratification of tumor specimens by virtue of their molecular characteristics. Such patterns will be helpful to detect previously unappreciated but important molecular features of the tumors that may turn out to have diagnostic or prognostic utility. These can be analyzed using multi-parametric tools for analyzing multiple prognostic features (such as Cox regression analysis, or other methods of multiple regression analysis) or by using methods developed for cDNA microarray image analysis (for example, scanner and image analysis software as described in U.S. Pat. No. 6,004,755, herein incorporated by reference in its entirety).

Analysis techniques for observing and scoring the experiments performed on tissue microarray sections include a bright-field microscope, fluorescent microscope, confocal microscope, a digital imaging system based on a CCD camera, or a photomultiplier or a scanner, such as those used in the DNA chip based analyses. The entire slide can either be visualized at once (and then breaking this up to multiple smaller entities) or images may be acquired separately from each tissue spot.

These results show that the very small cylinders used to prepare tissue microarrays can in most cases provide accurate information, especially when the site for tissue sampling from the donor block is selected to contain histological structures that are most representative of tumor regions. It is also possible to collect samples from multiple histologically defined regions in a single donor tissue block to obtain a more comprehensive representation of the original tissue, and to directly analyze the correlation between phenotype (tissue morphology) and genotype. For example, an array could be constructed to include hundreds of tissues representing different stages of breast cancer progression (e.g. normal tissue, hyperplasia, atypical hyperplasia, intraductal cancer, invasive and metastatic cancer). The tissue microarray technology would then be used to analyze the molecular events that correspond to tumor progression.

A tighter packing of cylinders, and a larger recipient block can also provide an even higher number of specimens per array. Entire archives from pathology laboratories can be placed in replicate 500-1000 specimen tissue microarrays for molecular profiling. Using automation of the procedure for sampling and arraying, it is possible to make dozens of replicate tumor arrays, each providing hundreds of sections for molecular analyses. The same strategy and instrumentation developed for tumor arrays also enables the use of tissue cylinders for isolation of high-molecular weight RNA and DNA from optimally fixed, morphologically defined tumor tissue elements, thereby allowing correlated analysis of the same tumors by molecular biological techniques (such as PCR-based techniques) based on RNA and DNA. When nucleic acid analysis is planned, the tissue specimen is preferably fixed (before embedding in paraffin) in an alcohol based fixative, such as ethanol or Molecular Biology Fixative (Streck Laboratories, Inc., Omaha, Nebr.) instead of in formalin, because formalin can cross-link and otherwise damage nucleic acid. The tissue cylinder of the present invention provides an ample amount of DNA or RNA on which to perform a variety of molecular analyses.

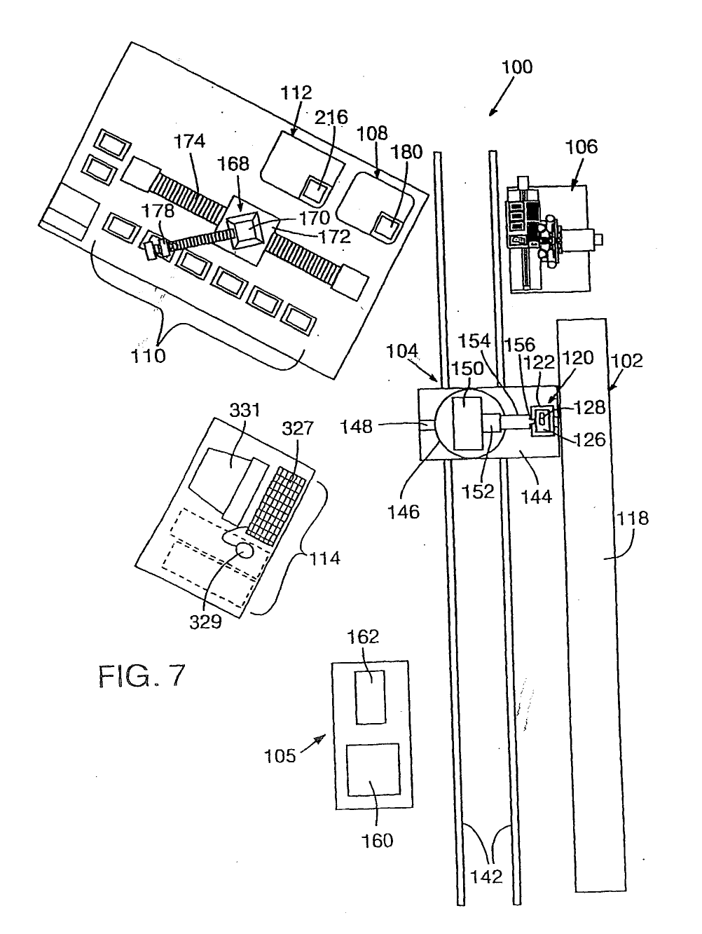

Embodiment of FIGS. 7-20

An example of an automated system for high speed preparation of the microarrays is shown in FIGS. 7-20. An overview of the system is illustrated in FIG. 7, which shows an automated apparatus 100 for preparing tissue specimens for analysis in microarrays. The apparatus includes a specimen source 102, a retriever 104 that retrieves tissue specimens from assigned locations in specimen source 102, and a detector 105 that locates a position of a tissue specimen within a specimen block and labels the specimen block with a computer readable identifier. Apparatus 100 further includes a constructor 106 that removes tissue samples from different tissue specimens and arrays the tissue samples in recipient blocks, a sectioner 108 that sections the blocks into sections, a reagent station 110 to which the sections are exposed, a scanner 112 for scanning the sections after they have been exposed to the reagents and obtaining digital images of the sections (and the component samples in the sections), and a controller 114. The controller 114 automatically controls the other components of apparatus 100, and records the identification of a subject associated with, a particular specimen, including clinical information about the subject.

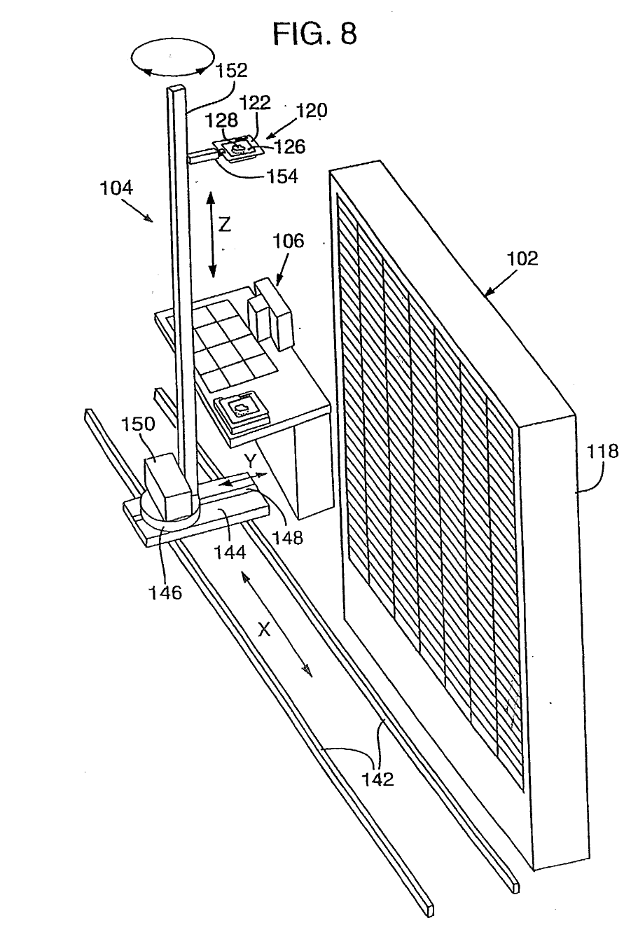

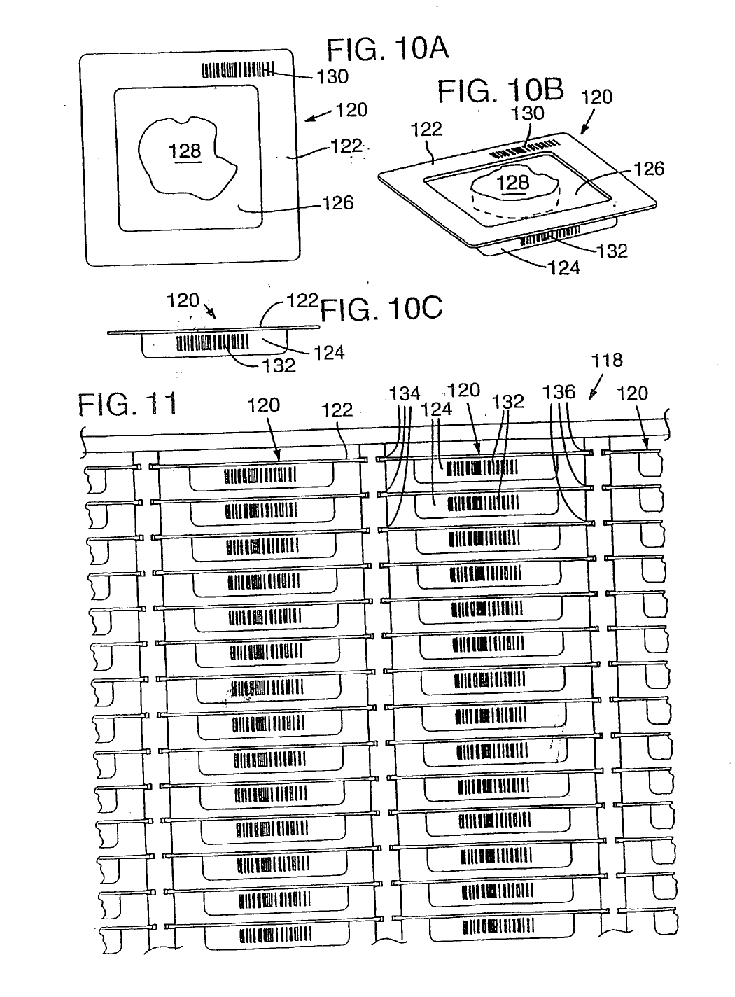

A particular embodiment of specimen source 102 is shown in greater detail in FIG. 8, which illustrates it as a cabinet 118 divided into many compartments that are arranged in columns and rows. Each of the compartments can be assigned a coordinate (e.g., x-y) identifier, so that a position of each of the compartments corresponds to a particular coordinate position within the columns and rows of compartments. As shown in FIGS. 10 and 11, each of the compartments is occupied by a specimen holder 120, which is formed by a peripheral flange 122 and a recessed bottom 124 that forms a central cavity which contains embedding medium 126 that contains a tissue specimen 128, such as a surgical pathological specimen of a tumor removed from a subject. A top surface of flange 122 is labeled with a first computer readable bar code identifier 130, and a side wall of bottom 124 is labeled with another copy of the computer readable bar code identifier 132.

As illustrated in FIG. 11, each compartment of cabinet 118 includes a pair of opposing, parallel slots 134, 136 which receives the lip of peripheral flange 122 to hold each specimen holder 120 in place within an assigned compartment. This arrangement allows each holder 120 to be slid into the compartment by aligning the edges of flange 122 with the slots 134, 136 and pushing the holder into the compartment. Alternatively, the holder 120 can be removed by pulling on it so that it slides along slots 134, 136 until the holder is disengaged from the compartment.

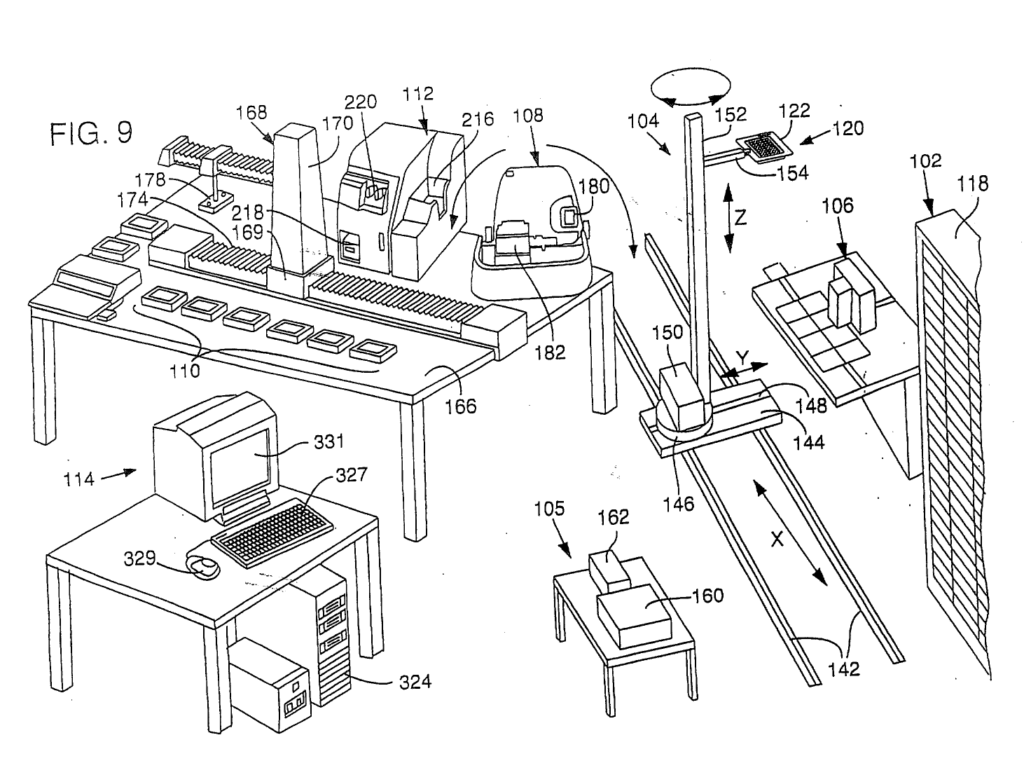

Holders 120 can be inserted into or removed from the compartments of cabinet 118 by the retriever 104 (FIG. 7), which in the disclosed embodiment is a robotic transporter (FIGS. 8, 9 and 12), which moves along a track 142 in an X direction, and which travels among the stations of apparatus 100, and permits the robotic arm access to all of the stations that it must reach. The robotic transporter includes a base 144 which supports a rotatable turntable 146, which in turn moves transverse to rails 142 (in a Y direction) along a guide channel 148. Mounted on turntable 146 is a motor 150 which moves retriever 104 along rails 142, rotates turntable 146, and moves turntable 146 in the Y direction along channel 148. Retriever 104 also includes an upright standard 152 mounted on turntable 146, and a retractable/extendible arm 154 that projects from standard 152. Arm 154 moves up and down standard 152 (in the Z direction illustrated in FIGS. 8 and 9). Retriever 104 therefore is capable of retrieving holders 120 from compartments of cabinet 118, and moving them in all three directions of movement (X, Y and Z) among the stations of apparatus 100.

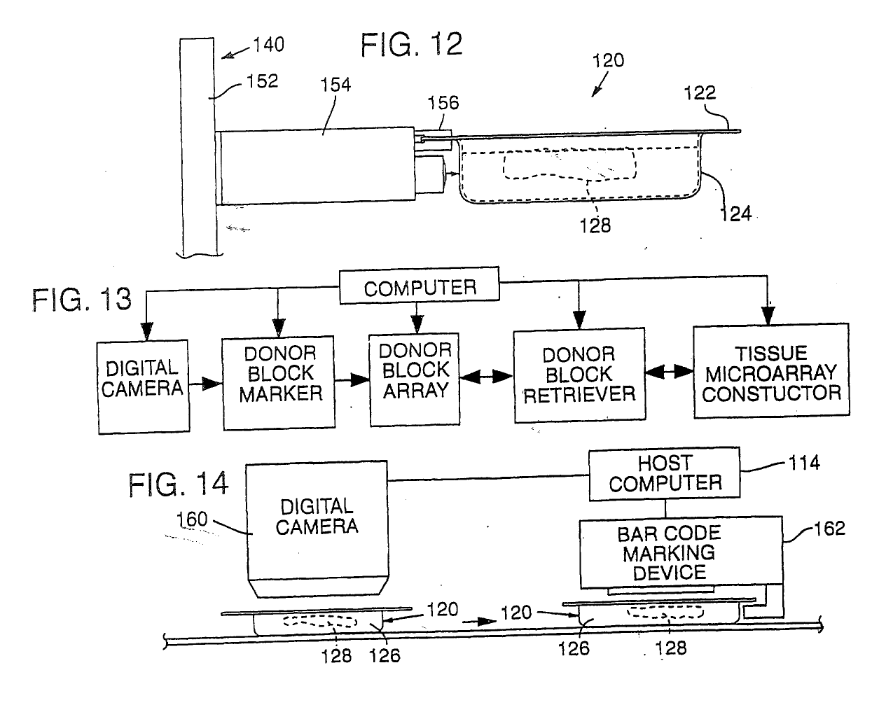

FIG. 12 illustrates an interaction between retriever 104 and holder 120. In this view, retractable arm 154 is shown fully retracted. At a free end of arm 154 is carried a clasp 156 with upper and lower jaws that fit above and below a front edge of flange 122 that is exposed when holders 120 are in place within the compartments of cabinet 118. Below clasp 156 is an optical reader 157 that is capable of reading bar codes displayed on a front of holder 120, and sending signals to controller 114 to identify tissue specimens contained in a holder.

FIGS. 7, 9 and 14 also illustrate a detector station, which includes a digital camera 160 and a bar code marker 162. As best illustrated in FIG. 14, digital camera 160 is capable of obtaining a digital image of tissue specimen 128 embedded in medium 126, to assign coordinates (such as x-y coordinates) to the outlines of specimen 128 with reference to a field defined by a surface of embedding medium 126 in holder 120. Alternatively, an operator could locate and mark (either by demarcating a region on the slide, or storing coordinates in a computer memory) regions of interest on the slide. This information could be electronically stored, to enable subsequent automated punching of samples from the tissue specimen. Coordinates within this region could subsequently be changed from “available” to “punched” once a sample has been punched from a site, such that a puncher would not subsequently attempt to obtain an additional sample from this site. Hence the region of interest defines a field within which potential donor sites are available.

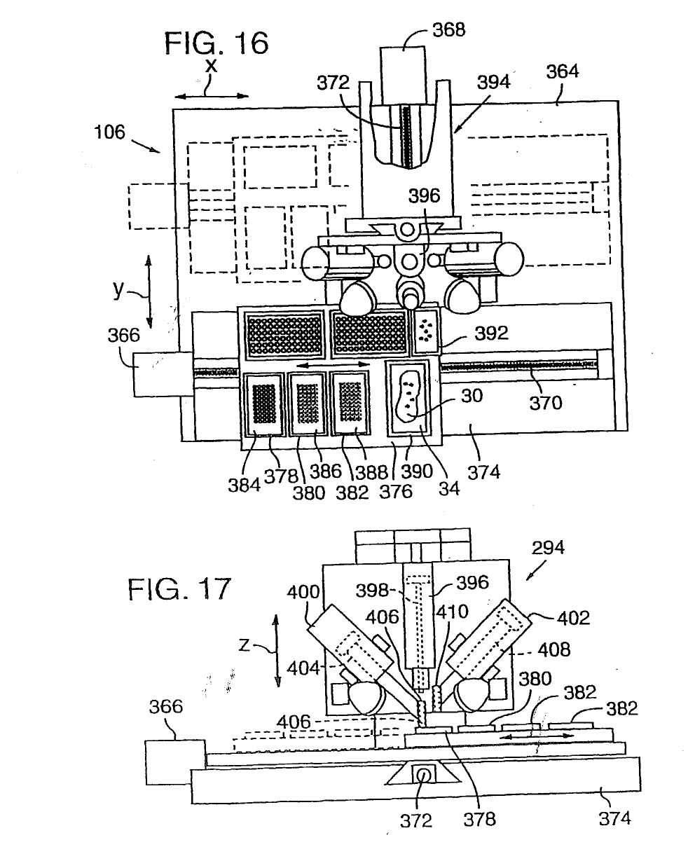

Tissue microarray constructor 106 is shown in FIGS. 7-9 and is discussed in greater detail in association with FIGS. 16-20 later in this specification.

Sectioner 108 is located on a table 166 (FIGS. 7 and 9), which also holds reagent station 110 and scanner 112. Also on table 166 is a robotic transporter 168 that can access all the stations on the table. Transporter 168 is of the type shown in U.S. Pat. No. 5,355,439, which is incorporated by reference. Briefly, the robotic transporter includes an upright standard 170 that is pivotally mounted on a base 172 that is capable of moving on elongated track 174. A cantilevered arm 176, which projects from near the top of standard 170, includes serrations along which a slide holder 178 is capable of moving.

Sectioner 108 on table 166 (FIGS. 7 and 9) is an automated, high speed microtome that includes an input port 180 into which recipient blocks can be placed, and an output port 182 (FIG. 9) from which sections of the recipient blocks are retrieved. An example of an automated microtome that could be used is found in U.S. Pat. No. 5,746,855, which is incorporated by reference. Reagent station 110 includes a series of reagent trays (such as solutions that contain nucleic acid probes or other markers for performing biological analyses such as detection of gene copy number alterations). An incubator setup for performing certain experiments as well as a washing station for removing unbound reagent, may be provided.

Scanner 112 on table 166 can be a scanner such as that shown in PCT publication WO 98/44333, which is incorporated by reference, and commercial embodiments available from Chromavision Medical Systems, Inc. of San Juan Capistrano, Calif. Most commercial microscopic imaging systems can be utilized for this purpose. Modifications may include automated stage capable of X-Y scanning and Z-axis autofocusing. A manual, user-defined image acquisition is also possible. Images may be acquired using a CCD camera, which may be computer controlled. Alternatively, the scanner can be a confocal scanner, such as described in U.S. Pat. No. 6,084,991. A Phosphorimager or a scanner of radioactive film (see Kononen et al., Nature Medicine 4: 844-847, 1998)_, can be used for quantitation of radioactive signal intensities, such as in mRNA in situ hybridization.

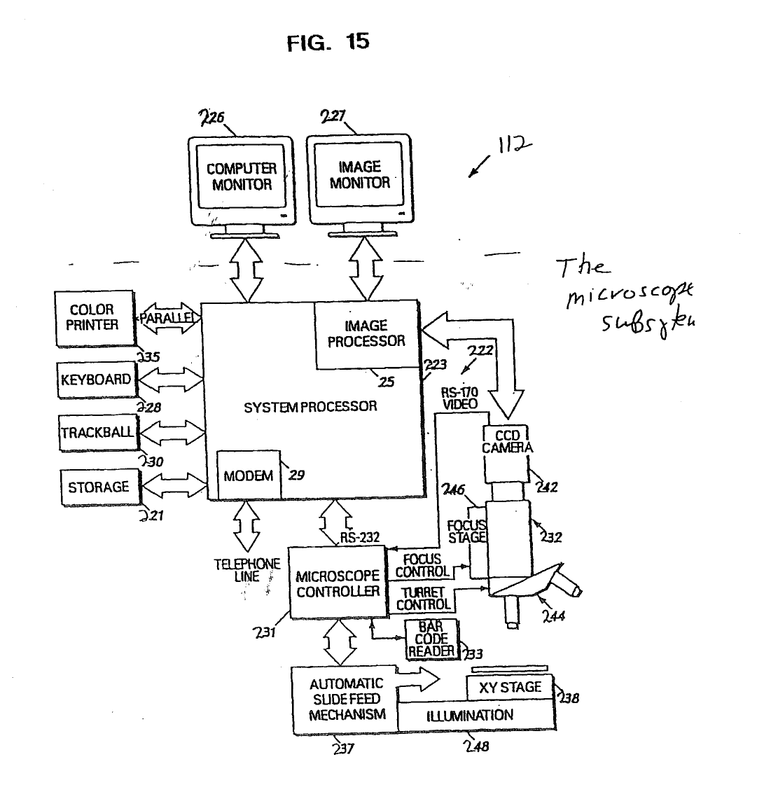

FIG. 15 shows a block diagram which illustrates scanner 112, which includes a microscope subsystem 232 housed in scanner 112. The scanner includes a slide carrier input hopper 216 and a slide carrier output hopper 218. A housing secures the microscope subsytem from the external environment. A computer subsystem includes a computer 222 having a system processor 223, an image processor 225, and a communication modem 229. The computer subsystem further includes a computer monitor 226 and an image monitor 227 and other external peripherals including storage device 221, track ball device 230, keyboard 228 and color printer 235. An external power supply 224 is also shown for powering the system.

Viewing oculars 220 of the microscope subsystem project from scanner 112 for operator viewing, although the system can be automated. Scanner 112 further includes a CCD camera 242 for acquiring images through the microscope subsystem 232. A microscope controller 231 under the control of system processor 223 controls a number of microscope-subsystem functions. An automatic slide feed mechanism 237 in conjunction with an X-Y stage 238 provides automatic slide handling. An illumination light source 248 projects light on to the X-Y stage 238 which is subsequently imaged through the microscope subsystem 232 and acquired through CCD camera 242 for processing in image processor 225. A Z stage or focus stage 246 under control of microscope controller 231 provides displacement of the microscope subsystem in the Z plane for focusing. The microscope subsystem further includes a motorized objective turret 244 for selection of objectives. This example is a bright-field microscope, but fluorescence micsorcopes and imaging systems may be similarly utilized.

Scanner 112 permits unattended automatic scanning of prepared microscope slides for the detection of candidate objects of interest, such as particular cells which may contain marker identifying reagents, and evaluation of the amount of the reagent that is present. Scanner 112 automatically locates candidate objects of interest present in a biological specimen on the basis of color, size and shape characteristics. Grades indicative of the amount of marker (such as a nucleic acid probe) are determined and summed to generate a score for the biological specimen. When the marker is a probe signal (as in fluorescent in situ hybridization or FISH) signal counting can be performed as in U.S. Provisional Patent application No. 60/154,601, which is incorporated by reference. This score may be used to evaluate whether the biological specimen contains a biological marker of interest. The system described in U.S. Provisional Patent Application No. 60/154,601 also includes description of a fluorescence microscope imaging system, which is widely applicable to analysis of other types of fluorescent stains.

An alternative example of signal counting and scoring would be performed as follows. Unattended scanning of slides is prompted by loading of the slides onto motorized X-Y stage 238. A bar code label affixed to each slide may be read by a bar code reader 238 to identify each slide during this loading operation. Each slide may be scanned at a low magnification, for example 20×, to identify potential samples that may display a positive signal (such as a FISH or IHC signal). After the low magnification is completed, the apparatus automatically returns to each candidate object, focuses at a higher magnification, such as 60×, and captures a digitized image for further analysis to confirm the object candidate. The degree of resolution required may depend on the type of analysis to be performed. If it is desirable to obtain information on the cellular or subcellular localization/distribution of the biomolecule, a high resolution is desirable. Quantitation of the overall fluorescence intensity or staining intensity in a tissue spot requires very little resolution, such as that obtained by a Phosphorimager used for radioactive detection.

A centroid for each confirmed cell candidate is computed and stored for evaluation of the marker. The marker can be the staining precicipate itself, or it can be a counter-stain. Scanner 112 then returns to the centroid for the first confirmed candidate object of interest and captures a color image of an area centered about the centroid. The pixel data for this area is processed to determine the amount of marker (for example as determined by intensity or hue of a color) in the area and a grade is assigned to the object. Scanner 112 continues processing and grading areas centered about other confirmed candidate objects of interest until a predetermined number of objects have been processed. An aggregate score is then computed from the grades for the predetermined number of objects. The object grades, aggregate score and images may then be stored in storage device 221, such as a removable hard drive or DAT tape, which communicates with controller 114. The stored images are available in a mosaic of images for further review. Alternatively, detected images can also be viewed directly through the microscope using oculars 227.

The images can be scanned in a variety of ways, for example by acquiring a montage image of the entire slide, and breaking it up into smaller segments each representing a single or multiple tissue spots or fractions thereof, or by acquiring a low-resolution scan of the slide, and then performing a high-resolution scan of each sample spot one at a time. This set of images could be subjected to morphological and image analytical tools to assess the quantity of immunostaining, based on the amount of staining present (as determined for example by intensity of the immunostain). This assessment could be based on the entire area in each sample spot, or by analysis of those regions in each tissue spot that contain tumor tissue, or other tissue of interest. The specific combination and strategy for image acquisition and analysis by a variety of factors, such as the number of specimens on the slide, their size, the type of staining or reagent system (brightfield or fluorescence), the number of parameters to be evaluated from the slide, the degree of automation required, the degree of resolution required, availability of autofocussing, CCD camera specifications, the desired instrumentation (microscope based, laser scanning, radioactive detection etc.).

An imaging system not only acquires images from a microscope slide, but may also archive, display, and analyze the images and incorporate data in a database. For example, the imaging system can display consecutive images on a computer screen for an observer to analyze, interpret and store. Alternatively, the program can pre-process images to display areas of positive staining, and present an image to the observer for approval. Completely automated image acquisition and analysis is possible. The image of a biomolecular marker detection may be compared with the corresponding Hematoxylin-Eosin stained morphological image (obtained from the same or nearby section) to verify that representative regions of the specimen are being evaluated.

Operating Environment for Controller (FIG. 21)

An exemplary operating environment for system controller 114 is shown in FIG. 21 and the following discussion is intended to provide a brief, general description of a suitable computing environment in which the invention may be implemented. The invention is implemented in a variety of program modules. Generally, program modules include routines, programs, components, data structures, etc. that perform particular tasks or implement particular abstract data types. The invention may be practiced with other computer system configurations, including hand-held devices, multiprocessor systems, microprocessor-based or programmable consumer electronics, minicomputers, mainframe computers, and the like. The invention may also be practiced in distributed computing environments where tasks are performed by remote processing devices that are linked through a communications network. In a distributed computing environment, program modules may be located in both local and remote memory storage devices.

Referring to FIG. 21, an operating environment for an illustrated embodiment of the present invention is a computer system 320 with a computer 322 that comprises at least one high speed processing unit (CPU) 324, in conjunction with a memory system 326, an input device 328, and an output device 330. These elements are interconnected by at least one bus structure 332.

The illustrated CPU 324 is of familiar design and includes an ALU 334 for performing computations, a collection of registers 336 for temporary storage of data and instructions, and a control unit 338 for controlling operation of the system 320. The CPU 324 may be a processor having any of a variety of architectures including Alpha from Digital; MIPS from MIPS Technology, NEC, IDT, Siemens and others; ×86 from Intel and others, including Cyrix, AMD, and Nexgen; 680×0 from Motorola; and PowerPC from IBM and Motorola.

The memory system 326 generally includes high-speed main memory 340 in the form of a medium such as random access memory (RAM) and read only memory (ROM) semiconductor devices, and secondary storage 342 in the form of long term storage mediums such as floppy disks, hard disks, tape, optical disks, CD-ROM, DVD-ROM, flash memory, etc. and other devices that store data using electrical, magnetic, optical or other recording media. The main memory 340 also can include video display memory for displaying images through a display device. Those skilled in the art will recognize that the memory 326 can comprise a variety of alternative components having a variety of storage capacities.

The input and output devices 328, 330 also are familiar. The input device 328 can comprise a keyboard 327, a mouse 329, a scanner, a camera, a capture card, a limit switch (such as home, safety or state switches), a physical transducer (e.g., a microphone), etc. The output device 330 can comprise a display 331, a printer, a motor driver, a solenoid, a transducer (e.g., a speaker), etc. Some devices, such as a network interface or a modem, can be used as input and/or output devices.

As is familiar to those skilled in the art, the computer system 320 further includes an operating system and at least one application program. The operating system is the set of software which controls the computer system's operation and the allocation of resources. The application program is the set of software that performs a task desired by the user, using computer resources made available through the operating system. Both are resident in the illustrated memory system 326.

For example, the invention could be implemented with a Power Macintosh 8500 available from Apple Computer, or an IBM compatible Personal Computer (PC). The Power Macintosh uses a PowerPC 604 CPU from Motorola and runs a MacOS operating system from Apple Computer such as System 8. Input and output devices can be interfaced with the CPU using the well-known SCSI interface or with expansion cards using the Peripheral Component Interconnect (PCI) bus. A typical configuration of a Power Macintosh 8500 has 72 megabytes of RAM for high-speed main memory and a 2 gigabyte hard disk for secondary storage. An IBM compatible PC could have a\Pentium PC with 1 Ghz processor with 526 Mb of RAM, 20-200 Gb of hard disk space. An exemplary Apple Macintosh may have a G4 600 MHz processor, 526 Mb of RAM, and 20-200 Mb diskdrive. Both may also house additional storage media, such as optical drives, CD-ROM (re-writable) and DVD-ROM, as well as backup systems.

In accordance with the practices of persons skilled in the art of computer programming, the present invention is described with reference to acts and symbolic representations of operations that are performed by the computer system 320, unless indicated otherwise. Such acts and operations are sometimes referred to as being computer-executed. It will be appreciated that the acts and symbolically represented operations include the manipulation by the CPU 324 of electrical signals representing data bits which causes a resulting transformation or reduction of the electrical signal representation, and the maintenance of data bits at memory locations in the memory system 326 to thereby reconfigure or otherwise alter the computer system's operation, as well as other processing of signals. The memory locations where data bits are maintained are physical locations that have particular electrical, magnetic, or optical properties corresponding to the data bits.

Particularly preferred data storage would be on optical disks, CD-R, CD-RW, or DVD-ROMs. A tissue microarray storage requirement is often about 2 Gb per slide when images are acquired with a high-resolution CCD camera. This will take half of the available storage space on a DVD-ROM. Compression of images may be required for storage of images or displaying them for observer at the time of image interpretation or future review.

System Operation

The operation of the system is best illustrated in FIGS. 7-9. Specimen holders 120 are placed in cabinet 118 of specimen source 102 by inserting the peripheral flange 122 of each holder 120 into opposing slots of each compartment. The position of each specimen in the matrix of compartments is recorded, and associated with identifying information (such as patient identity and clinical information) about tissue specimen 30 in the holder. Each holder 120 may be retrieved from its compartment by retriever 104, which moves along rails 142 to position standard 152 in front of a first column of compartments in cabinet 118. Arm 154 is then extended, until the jaws of clasp 156 are positioned above and below a front lip of peripheral flange 122 of holder 120, and the clasp is actuated to grip the peripheral flange. Arm 154 is then retracted to pull holder 120 from its compartment, transporter 104 then rotates on turntable 146 as it travels down rails 142 in the X direction toward detector station 105.

Once transporter 104 has reached detector station 105, arm 154 is moved in the Z and Y directions to position holder 120 below digital camera 160. The digital camera then obtains a digital image of specimen 128 in embedding medium 126, and determines x-y coordinates of specimen 128 relative to holder 120 which are recorded by controller 114. Holder 120 is then transported by arm 154 to bar code marker 162, where computer readable bar code labels 130, 132 (see FIG. 10) are applied to the top flange 122 and side face of holder 120. These bar code labels are uniquely associated with the holder from a particular compartment in the cabinet 118, which is in turn associated with identifying information about the specimen in the holder (including the location of specimen 128 in holder 120).

Holder 120 is then retrieved from station 105 (FIGS. 7 and 9) by transporter 104, and may be returned to an assigned compartment in cabinet 118 (such as the compartment from which it had been previously retrieved). Alternatively, transporter 104 can convey the holder, to which the bar codes have been applied, to constructor station 106 where samples are removed from the sample in the holder and placed in recipient blocks (such as blocks 50-54 in FIG. 2). The operation of constructor station 106 is more fully described in association with FIGS. 16-20.

After each recipient block is formed, it is placed back in the labeled holder 120, which is lifted by arm 154 off of constructor station 106. Transporter 104 then rotates, and empties the recipient block in tray 120 into input port 180 (FIGS. 7 and 9) of sectioner 108. The block is sectioned, each of the sections is mounted on a rigid support (such as a glass slide) that is labeled with a bar code which corresponds to the bar code on holder 120 from which the block came. The sections are then retrieved by robotic transporter 168, and exposed to bioanalysis reagents in reagent station 110 (such as solutions that contain nucleic acid probes for informative biological markers). Exposure to various reagents can be performed, for example, as described in U.S. Pat. No. 5,355,439, which is incorporated by reference. Once these reactions have been performed, each section is then transported by robotic transporter 168 to input hopper 216 of automated scanner 112, where each section is scanned to determine whether any of the samples on the section provide biologically useful information. For example, the scanner would determine whether a color change has occurred that would indicate hybridization of a nucleic acid probe to the sample, and would quantify such a color change to determine changes in gene copy number.

In addition to sectioning, the tissue microarray constructor may obtain samples of tissues for cell free analyses. For example, the presence or expression of a gene or mutant gene may be determined in a tissue sample by a broad range of of cell free techniques known in the art, such as protein immunoblotting, immunoprecipitation, Northern blot, RT PCR, single-strand confirmational polymorphism, serial analysis of gene expression, differential display and the like. Tissue samples may be obtained from a particular type of normal or diseased tissue, to perform a cell free analysis of one or more biomarkers in that tissue. For example, one may obtain a tissue sample or series of samples from a particular type of carcinoma to perform serial analysis of gene expression, differential display, or other high throughput analysis of gene expression.

Tissue samples may also be obtained from specific regions of interest (ROI) in a tissue specimen. The samples of the region of interest may then be used in a cell free analysis of one or more biomarkers. Tissue samples from similar or dissimilar regions of interest may be pooled if desired, for cell free analysis of biomarkers or biomolecules in the pooled tissue. Such analysis may help to define the molecular nature of a region of interest. For example, comparisons of mutations and/or gene expression could be made between ROIs representing invasive carcinoma and ROIs representing carcinoma in situ. As another example, comparisons could be made between ROIs representing different stages of development of an atheroma in a blood vessel. As another example, comparisons could be made of different stages of development in a particular tissue (e.g., in utero development of a mouse heart, comparisons of heart muscle obtained from young and old subjects, etc.), In this way, molecular changes associated with tissue or organ development or aging could be investigated. The same kinds of analysis may of course be performed on tissue specimens without obtaining samples from specific regions of interest. However, performing these analyses on specific regions of interest may considerably enhance sensitivity, specificity and/or utility of observations.

Digital images of the samples on each section, or at least samples that are determined to be of interest, are then stored in controller 114 for future reference. The stored information may include the actual image itself, as well an any quantitative data acquired from the image. This information is incorporated into a database, and could later be retrieved and examined for correlation between clinical findings and biological findings in the digital images. The biological findings can be automatically determined. Alternatively, the images can be scored by a pathologist or other examiner, by calling up the electronic images to score on a screen, rather than at a microscope. The examiner can click on a menu which provides possible interpretations, save the data, and move on to the next image. In this manner, a large number of tissue samples, for example samples from a large number of specimens from different subjects, can be quickly scored.

The sections themselves may be returned to compartments in cabinet 118, or discarded. Hence the entire operation can be automated, and performed continuously, for high throughput analysis of many thousands of slides in a single day. This automated apparatus therefore obtains potentially millions of data points about the reactions of different samples on different slides from different tissue specimens with different reagents, which can then be analyzed. The parallel analysis of many different data points permits an appreciation of previously unrecognized biological associations between different tissue specimens (such as gene amplifications in tumors of similar stage or grade). Previously unrecognized differences between different tissue specimens can also be demonstrated, such as changes in gene copy number at different stages of tumor progression.

Operation of the Tissue Microarry Constructor (FIGS. 16-20)

An example of an automated tissue microarray constructor 106 is shown in FIGS. 16-20. Constructor 106 includes a stage 364 having an x drive 366 and a y drive 368, each of which respectively rotates a drive shaft 370, 372. The shaft 372 moves a specimen bench 374 in a y direction, while the shaft 370 moves a tray 376 on bench 374 in an X direction. Mounted in a front row of tray 376 are three recipient containers 378, 380 and 382, each of which contains a paraffin recipient block 384, 386 or 388, and a donor container 390 that contains tissue specimen 30 in embedding medium 34. In a back row on the tray is a discard container 392.

Disposed above stage 364 is a punch apparatus 394 that can move up and down in a Z direction. Apparatus 394 includes a central, vertically disposed, stylet drive 396 in which reciprocates a stylet 398. Apparatus 394 also includes an inclined recipient punch drive 400, and a inclined donor punch drive 402. Punch drive 400 includes a reciprocal ram 404 that carries a tubular recipient punch 406 at its distal end, and punch drive 402 includes a reciprocal ram 408 that carries a tubular donor punch 410 at its distal end. When the ram 404 is extended (FIG. 17), recipient punch 406 is positioned with the open top of its tubular bore aligned with stylet 398, and when ram 408 is extended (FIG. 19), donor punch 410 is positioned with the open top of its tubular bore aligned with stylet 398.

The sequential operation of the apparatus 394 is shown in FIGS. 17-20. Once the device is assembled as in FIG. 16, a computer system (such as controller 114) can be used to operate the apparatus to achieve high efficiency. Hence the computer system can initialize itself by determining the location of the containers on tray 376 shown in FIG. 16. The x and y drives 366, 368 are then activated to move bench 374 and tray 376 to the position shown in FIG. 17, so that activation of ram 404 extends recipient punch 106 to a position above position (1,1) in the recipient block 384. Once punch 406 is in position, apparatus moves downward in the Z direction to punch a cylindrical bore in the paraffin of the recipient block. The apparatus 394 then moves upwardly in the Z direction to raise punch 406 out of recipient block 384, but the punch 406 retains a core of paraffin that leaves a cylindrical receptacle in the recipient block 384. The x-y drives are then activated to move bench 374 and position discard container 392 below punch 406. Stylet drive 396 is then activated to advance stylet 398 into the aligned punch 406, to dislodge the paraffin core from punch 406 and into discard container 392.

To receive the paraffin core, discard container 392 may have an open top, or a closed top with holes 393 of inside diameter slightly larger than the punch outside diameter. Punch 406 is lowered into hole 393, stylet 398 is depressed, and released, and punch 406 raised so distal end of punch is just slightly above discard container 392. X-y drives 396, 398 move the bench (which includes discard container 392 so the punch tip is no longer over the hole and any paraffin stuck to the punch tip is knocked off. Discard container contains multiple holes 393 for different size punches.

Alternatively, paraffin core from recipient block can be inserted into donor block in a location from which a tissue sample had been previously extracted. This can provide additional structural strength to the donor block when many punches are taken from the same general area of a specimen.

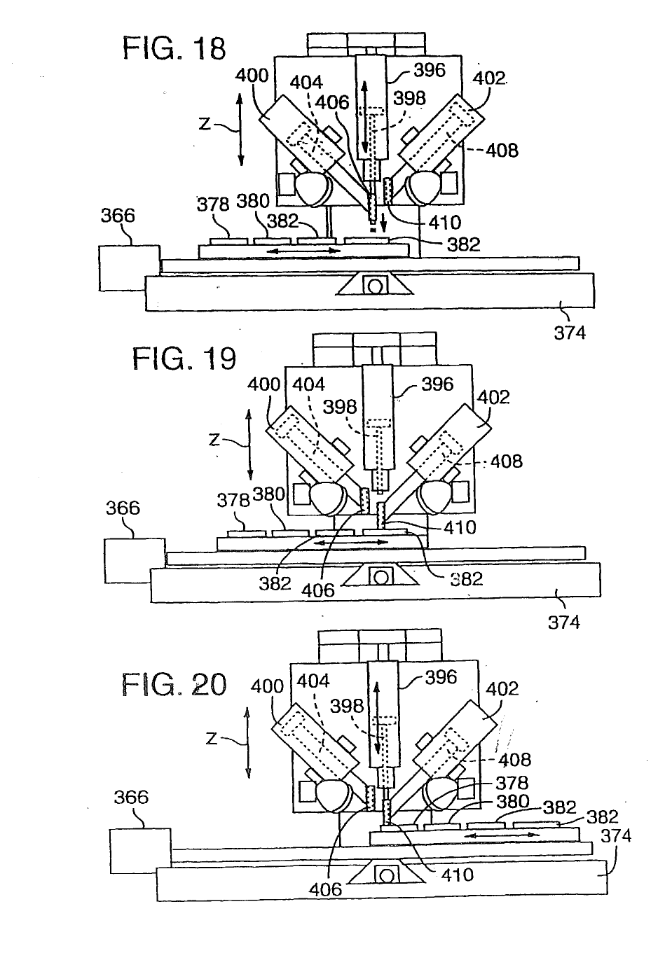

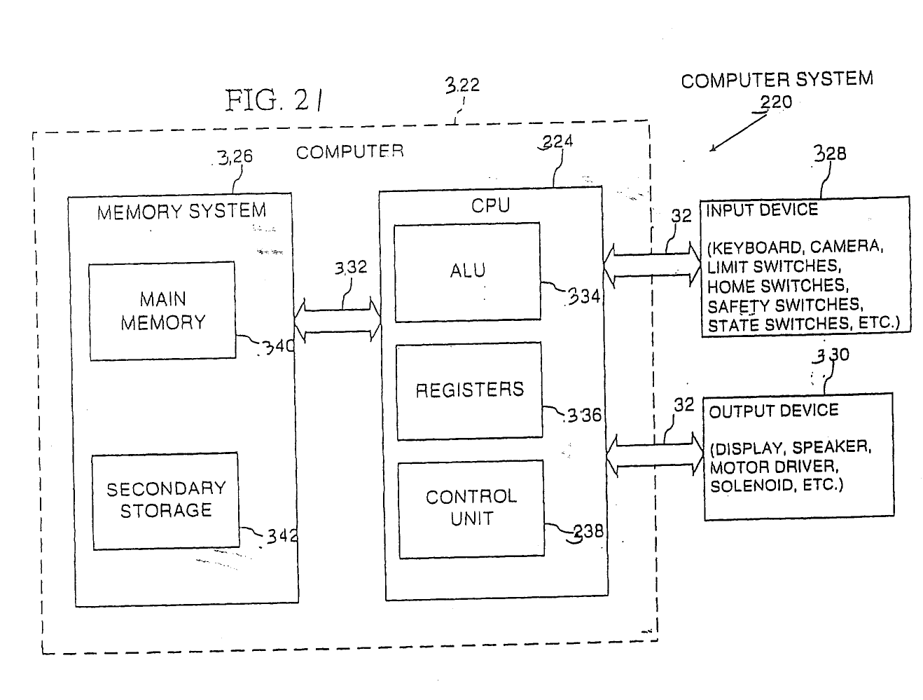

Stylet 398 is retracted from recipient punch 406, ram 404 is retracted, and the x-y drive moves bench 374 and tray 376 to place donor container 390 in a position (shown in FIG. 18) such that advancement of ram 408 advances donor punch 410 to a desired location over the donor block 34 in container 390. Apparatus 394 is then moved down in the Z direction (FIG. 19) to punch a cylindrical core of tissue sample out of the donor block 34 and apparatus 394 is then retracted in the Z direction to withdraw donor punch 410, with the cylindrical tissue sample retained in the punch. The x-y drive then moves bench 374 and tray 376 to the position shown in FIG. 20, such that movement of apparatus 394 downwardly in the Z direction advances donor punch 410 into the receptacle at the coordinate position (1,1) in block 384 from which the recipient plug has been removed. Donor punch 410 is aligned below stylet 398, and the stylet is advanced to dislodge the retained tissue sample cylinder from donor punch 410, so that the donor tissue cylinder remains in the receptacle of the recipient block 386 as the apparatus 394 moves up in the Z direction to retract donor punch 410 from the recipient array. Ram 408 is then retracted.

This process can be repeated until a desired number of recipient receptacles have been formed and filled with cylindrical donor tissue samples at the desired coordinate locations of the array. Although this illustrated method shows sequential alternating formation of each receptacle, and introduction of the tissue cylinder into the formed receptacle, it is also possible to form all the receptacles in recipient blocks 384, 386 and 388 as an initial step, and then move to the step of obtaining the tissue samples and introducing them into the preformed receptacles. The same tissue specimen 30 can be repeatedly used, or the specimen 30 can be changed after each donor tissue specimen is obtained, by introducing a new donor block 34 into container 390. If the donor block 34 is changed after each tissue cylinder is obtained, for example, each coordinate of the array will include tissue from a different tissue specimen.

One or more recipient blocks 384 can be prepared by placing a solid paraffin block in container 378 and using recipient punch 106 (FIGS. 17-18) to make cylindrical punches in block 384 in a regular pattern that produces an array of cylindrical receptacles. The regular array can be generated by positioning punch 406 at a starting point above block 384 (for example a corner of the prospective array), advancing and then retracting punch 406 to remove a cylindrical core from a specific coordinate on block 384, then dislodging the core from the punch by introducing a stylet into opening 407. The punch apparatus or the recipient block is then moved in regular increments in the x and/or y directions, to the next coordinate of the array, and the punching step is repeated.

Any or all of the operation of a tissue microarray constructor may be controlled by a controller such as a computer. This includes any and all of the processes illustrated in FIG. 16-20. The controller may, for example, control movement of stage 364 by controlling x drive 366 and y drive 368; control operation and alignment of punch apparatus 394, such as controlling location of punch sites and depth of punch sites; control operation of stylet 398 to eject tissue sample and/or paraffin core; control detection and proper positioning of donor and recipient blocks; control placement of tissue sample into an assigned receptacle in recipient block 386; control operation and alignment of discard container 392 with stylet 398 and punch 406. Other functions which may be controlled by the controller include detection of damaged punches, and detection of block surfaces in relation to punch.

The controller allows an operator to completely design an array for automated construction by the tissue microarray constructor. An operator can specify construction of an array by indicating, for example: the specific donor tissue specimen to be sampled; the region of interest in the donor tissue specimen to be sampled; location of donor tissue specimen placement in recipient block; and size, shape, and regularity of the microarray, for example the total number of rows and columns of tissue specimens in the recipient block.

In the specific disclosed embodiment, the cylindrical receptacles of the array have diameters of about 0.6 mm, with the centers of the cylinders being spaced by a distance of about 0.7 mm (so that there is a distance of about 0.05 mm between the adjacent edges of the receptacles). Although the diameter of the biopsy punch can be varied, 0.6 mm cylinders have been found to be suitable because they are large enough to evaluate histological patterns in each element of the tumor array, yet are sufficiently small to cause only minimal damage to the original donor tissue blocks, and to isolate reasonably homogenous tissue blocks. Up to 1000 such tissue cylinders, or more, can be placed in one 20×45 mm recipient paraffin block. Specific disclosed diameters of the cylinders are 0.1-4.0 mm, for example 0.5-2.0 mm, and most specifically less than 1 mm, for example 0.6 mm. Computer-guided placement of the specimens allows very small specimens to be placed tightly together in the recipient block's array of receptacles. For example, a 0.4 mm punch diameter would allow construction of an array with 0.5 mm center to center distance between specimen cores, thereby increasing the number of specimens that can be obtained from a 15×15 mm tissue area to 900.

FIG. 3B shows the array in the recipient block after the receptacles of the array have been filled with tissue specimen cylinders. The top surface of the recipient block may be covered with an adhesive film from an adhesive coated tape sectioning system (Instrumedics) to help maintain the tissue cylinder sections in place in the array once it is cut. The array block may be warmed at 37° C. for 15 minutes before sectioning, to promote adherence of the tissue cores and allow smoothing of the block surface when pressing a smooth, clean surface (such as a microscope slide) against the block surface.

Marking and Obtaining Regions of Interest in a Tissue Sample

Tissue samples generally contain multiple cell types. For example, a sample which contains a breast cancer may often have regions of surrounding stroma and connective tissue as well as atypical and normal epithelial tissue, in addition to regions of in situ (noninvasive) carcinoma or invasive carcinoma. Inflammatory cells, such as infiltrating lymphocytes and other leukocytes are common, as are areas close to necrotic regions. The cancer components of the tumor may have a varying degree of differentiation, or other morphological differences. The stromal, connective tissue and atypical and normal appearing epthelium may have subtle genetic differences or they may be having reactive changes to the growth factors, inflammation etc. produced by the tumor. Depending on the particular application of the microarray technology, the region of interest (ROI) may be any of these regions or all of these regions. Indeed, it is possible and usually desirable to define multiple histologic and pathologic features in a sample. Thus, a ROI is any subset of a tissue sample or tissue section which contains any feature or features to be imaged, examined or studied. The ROI subset may be the entire tissue sample or the entire tissue section, or any portion or portions of the tissue sample or tissue section.

It is often desirable to have tissue samples used in construction of a microarray marked to define one or multiple ROIs. The tissue microarray constructor's controller may be used to guide the tissue microarray constructor to obtain tissue samples from these ROIs, and place the ROI tissue samples into a recipient block microarray.

To accomplish this, the ROI is determined and marked in a manner that allows future automated retrieval of a tissue sample from the ROI. The marking method may include two separate stages. The first stage provides a method for ROI marking of a slide image (or any other image of the tissue donor block) and storing the information either in a standalone file or a database. The second function provides a method for regenerating the ROI perimeters using the stored information from the stage, and generating tissue microarrayer punch locations within the ROI. Each stage can be enhanced to provide the user more options and flexibility.

Determining and Marking Regions of Interest

Regions of interest (ROIs) within a tissue sample or specimen are determined, for example, by examining a section from a tissue donor block. An optical or digital image of the section is acquired through any suitable method (for example a high-speed CCD camera attached to a microscope), and ROIs are marked by an observer. Alternatively, ROIs may be marked on a digital image of the tissue specimens. The location of the ROIs are represented digitally as data points that can be stored and later used to regenerate perimeters of the ROIs.

Methods for marking ROIs are provided. For example, an observer may use a pen to manually mark a ROI in a sample on a microscope slide. A digital image of the sample is obtained, and the user may trace over the lines defining the ROI. Alternatively, detection of the ROI lines may be automated. If there are no markings present to identify the ROI, the user identifies ROIs on an image and marks them directly, for example marking digitally on a digital image. The user can annotate the ROIs with tissue type or other properties, characteristics, or instructions. These annotations can be automatically linked with the recipient tissue microarray block to which tissue from the ROI is transferred.

Once a digital image of an ROI is obtained, an estimate of the amount of tissue available in each ROI can be calculated, for example by multiplying a visible top surface area of tissue by an anticipated or measured depth of the specimen. The number of punches which can be extracted from each ROI can be calculated, by dividing the calculated volume of available tissue by a volume of each punch. By referring to annotations, the total amount of a particular kind of tissue in a tissue donor block, or in the entire tissue donor block array, can also be calculated.

Additional approaches to defining the ROI may make use of such features as distance from a particular distinctive area. The distinctive area may be a necrotic area, a tumor-stromal boundary, or any observable feature. Immunostaining or in situ hybridization or other biological reagent could be used to stain a section of a microarray, and then target the correponding region in a block for tissue microarray construction. For example, tissue microarrays could be constructed from regions that stain negative and positive for a particular immunostain, such as estrogen receptor-positive and estrogen-receptor negative regions.

Additional information is included to provide accurate marking information independent of the marked perimeters, scaling and orientation. This additional information can be provided by the use of indicia such as reference points. The use of such external reference marks aids in correcting for the effects that derive from the expansion, contraction and other morphological distortion that accompanies the sectioning of a tissue block, as well as the staining, and fixation of the tissue section on the slide. An indicium such as a reference point is in approximately the same position in the original tissue block as it is in a slide representing the same tissue block. For example, if a slide were placed on top of the source tissue block in the appropriate orientation, the indicia or reference points of the block would align with those on the slide.

Such alignment may be achieved, for example, as illustrated in FIG. 29A by embedding an indicium or indicia in a tissue donor block 500 before sectioning. The embedded indicia or reference points 504 may be fluorescent, magnetic, or in some other way distinctive from the surrounding tissue 502 and block substrate material 503, to facilitate detection in subsequent construction of tissue microarrays. The examples of indicia in FIG. 29A are elongated, and extend through block 500 in a direction that intersects the direction of the section cuts through block 500. As illustrated in FIG. 29B, the indicia or reference points 506 are sectioned during sectioning of the tissue donor block, and maintain substantially the same position with respect to the tissue section 508 on a slide 510 as they have to the tissue sample in the tissue donor block. The ROI perimeter 512 may then be defined, and stored if desired, as a function of distance from the reference points. As an alternative, the reference points may not maintain a same position with respect to a tissue, but may vary in a predictable manner on different sections that allows the reference points to help locate ROIs or other structures in the tissue.

Reference points may be used to control for scaling and orientation. For example, the ROI may have been originally marked on an image much larger than the actual size of the tissue in the tissue donor block. In addition, the section used to define the ROI may be mounted on the slide in any rotational orientation. If the stored digital image information includes reference point information (including the actual distance between them in the tissue donor block), both scaling and orientation can be readily accounted for prior to taking a new sample for microarray construction.

Information on ROIs in tissue donor blocks can be stored in a database. In addition to the ROI perimeters and reference point information, the database could include substantial rotations, including patient demographic data, nature of disease process or tumor, characteristics of the ROI (for example, a region of well-differentiated carcinoma, invasive poorly differentiated carcinoma, etc). Other information in the database could include, for example, location of the tissue donor block in a tissue donor block array, location of the sample in the recipient array, and/or location of the recipient block array. The reference points can be composed of any material, such as human or animal tissues, cells or other biological material, either stained or unstained. It may include stains embedded in any medium. Stains could be bright-field or fluorescent. Stains may be mixed with an appropriate medium chemically or they may be beads that are embedded in the material made into a reference point format.

The reference points may be, for example, inserted to the vicinity of the tissue before sections are obtained. A convenient method is drilling, punching or otherwise inserted in a paraffin block after the block has been fully processed.

Obtaining a Sample from a Region of Interest

Given the stored coordinates of the ROI, the tissue microarray constructor extracts a tissue sample from the ROI in a tissue donor block (as described in Operation of the Tissue Microarray Constructor, above). The tissue microarray constructor deposits the sample from the ROI in a specified location in a recipient block.

To accomplish this task, the tissue microarray constructor can use at least two microarray constructor reference points, of known separation distance and known angle in reference to the arrayer coordinates. If the absolute coordinates of one of the two microarray constructor reference points is known, and the microarray constructor reference points are placed in the field of view of an imaging system associated with the tissue microarray constructor, the microarray constructor reference points appear in the same image as the tissue donor block.

The tissue microarray constructor's imaging system can also detect at least two tissue donor block reference points (for example, embedded in paraffin in the tissue donor block). The imaging system detects the microarray constructor and donor block reference points, and delivers the information to the controller which electronically retrieves the stored ROI information, and regenerates the ROI perimeters given the known identity of the tissue donor block and its reference point locations. The distance between the reference points on the block image is compared to the distance between the reference points on the slide image (which is stored along with ROI information). The comparison yields a scaling factor which can be used to scale all the ROI saved information and display it in the correct scale on the block image. The controller generates the desired punch locations inside the ROIs and translates these punch locations to the tissue microarray constructor. The punch then obtains the sample and places it into a recipient block microarray (as described in Operation of the Tissue Microarray Constructor, above).

The conveniences of being able to move punch locations around, delete certain punches, mark certain punches as undesired, add punches, etc. are also provided to the user at this stage. Changes that occur to the tissue donor block due to tissue extraction are stored in the database. Consequently, if the tissue at a certain location was extracted and deposited in a recipient block, then information about the position of extraction is stored in the database to substantially prevent a user from attempting to mark the same position for extraction at a later point.

A minimum of two donor block reference points can be used to accurately reconstruct the ROI perimeters. More than two donor block reference points, for example three or more reference points, may enhance the accuracy with which the ROI is located during subsequent microarray construction. Having three or more reference points in the tissue donor block also ensures that if a reference point is lost during sectioning, two or more would remain on the slide to assist in the block marking process. It is possible to derive multiple different regions within a block. High-resolution imaging of a slide combined with accurate alignment with the block would allow sub-millimeter accuracy in the region of the punch for tissue microarray construction.

Recipient Array Design

Scaling information enables estimates of tissue quantities of a specific tissue type to be calculated. An array of similar or different tissue types may be composed by defining the layout or arrangement of the array within the recipient block (for example a 4 by 6 subarray of a particular tissue type, a 5 by 5 array of a different tissue type, etc.). The punching properties are specified for both the tissue donor block and the recipient block. For example, the punch size and punch spacing may be specified for both tissue donor block and recipient block. The database can be examined to determine which tissue donor blocks satisfy the request along with information on where to extract tissue and how much to extract from each individual tissue donor block. Once tissue is extracted, the database is updated, for example to include information on amount and location of tissue removed from the tissue donor block, and tissue location information within the recipient array.

Database queries can be submitted remotely, and an operator does not need to be physically near the tissue microarray constructor. Construction of tissue microarrays can be entirely automated, and does not require operator intervention other than defining the ROI and specifying the composition of a particular microarray.

Additional information regarding regions of interest is presented in Example 7.

Reagent Station

Once recipient tissue microarray blocks are sectioned by sectioner 108, and the tissue microarray sections are mounted onto microscope slides, they may be prepared for a variety of subsequent analyses. These analyses may include, for example: detection of tissue micro-structure with, for example, hematoxylin/eosin (H&E) staining; detection of specific gene expression at the mRNA level with, for example, in situ hybridization; detection of specific gene expression at the protein level with, for example, immunohistochemistry (IHC); detection of genetic abnormalities at the DNA level, with, for example, fluorescence in situ hybridization (FISH); detection of specific enzymatic activities in tissues, with, for example, histochemistry (e.g., NADPH diaphorase histochemistry to detect nitric oxide synthase activity); and detection of apoptotic cell death, with, for example, TUNEL assay. Any other staining that can be done on regular sections can be done on tissue microarray sections.

Each of these analyses can be performed with a specific series of steps performed in a defined order, often with a need for precise timing. For example, paraffin embedded tissue microarray sections may be prepared for subsequent immunohistochemical analysis by incubation at 37° C., followed by xylene treatment (two changes, three minutes each); rehydration by passing through graded alcohols (two changes, absolute ethanol, three minutes each, followed by two changes, 95% ethanol, three minutes each); followed by a water rinse.

After preparation, the sections are incubated for a defined period of time (typically 30 minutes to two hours) with a dilute solution of antibody (for example, antibody ER ID5, anti-human estrogen receptor monoclonal antibody from DAKO, Glostrup Denmark, at 1:400 dilution in phosphate buffer saline (PBS)+3% bovine serum albumin (BSA)). The slide is washed three times with PBS, a secondary antibody is applied (for example, biotinylated anti-mouse IgG, 1:1000 dilution in PBS+3% BSA for 30 min), the slide is washed with PBS, avidin-biotinylated horseradish peroxidase complex is applied for thirty minutes, and the slide again washed with PBS. In this example, the presence of estrogen receptor in tissue microarray sections may be detected by applying diaminobenzidine solution to the slide, and observing the slide for the presence of brown color. The intensity and distribution of the colorimetric reaction may be quantified by image analysis.

The present invention includes a series of individual reaction chambers at reagent station 110 (FIGS. 7 and 9) at which such timed steps are performed. A conveyor or robotic arm moves the tissue microarray sections between the reaction chambers according to instructions delivered by the controller 114 of this computer implemented system. After individual section of the blocks emerge from output port 182 of sectioner 108, robotic transporter 168 can individually deliver different sections to different reagent trays in reagent station 110. The processing may include washing, fixing and embedding a section. Processes can be temperature and humidity controlled. Multiple commercially available reagent preparation stations are available that perform either a complete processing of microascope slides, or a specific step (such as hybridization, staining or incubation).

The sections mounted on slides are transported via robotic arm 168 from microtome 108 to individual workstations of the reagent station 110. At each workstation, successive specific, timed procedures may be performed (for example, deparaffinization by warming the slide to 37° C., followed by xylene treatment; passage through graded alcohols; rinsing in water). The movement of each slide by robotic arm 168, and its timing at each position, are controlled by instructions entered by the operator into host the computer of controller 114. Sectioner 108 applies a bar-code marker to each slide to identify it, so that the robotic arm will be able to identify each slide. For example, the bar code may identify the slide as containing an array of specific breast-cancer sections, which are to be processed through a series of workstations optimized for the detection of estrogen receptor expression.

Once slides are prepared, robotic arm 168 may transfer them to scanner 112 for image analysis, as already described. Image analysis yields quantitative data regarding presence, amount, and distribution of a particular set of biological markers within cells, between cells in a specimen, within tissue spots and between tissue spots. This is stored in a database, along with tissue specimen identity (for example, breast biopsy), clinical information regarding the patient (for example, age, sex, medical history, family history, social history, physical findings, laboratory values), tumor-node-metastasis staging and/or stage grouping, histologic tumor subtype, nature of treatment given, clinical course and response to therapy, and any other relevant information available. The database would also store location information (for example, coordinates of the tissue specimen in donor block, location of the donor block in the donor block array, location of recipient block in recipient block array).

The database's power as a scientific and clinical tool increases with the amount and reliability of the stored information. For example, medical professionals may enter detailed medical histories and other clinical data directly into remote computers, which would transmit that information directly to the database. Such information would allow continuous updating of clinical information, which would then be correlated with quantitative data from an increasing number of biological markers. In addition, an accurate, thorough, and up-to-date database would allow investigators to identify new biological markers and assess disease pathogenesis, or their value in prognosing disease or predicting response to therapeutic interventions.

Examples of Array Technology

Applications of the tissue microarray technology are not limited to studies of cancer, although the following Examples disclose embodiments of its use in connection with analysis of neoplasms. Array analysis could also be instrumental in understanding expression and dosage of multiple genes in other diseases, as well as in normal human or animal tissues, including tissues from different transgenic animals or cultured cells.

Tissue microarrays may also be used to perform further analysis of genes and targets discovered from, for example, high-throughput genomics, such as DNA sequencing, DNA microarrays, or SAGE (Serial Analysis of Gene Expression) (Velculescu et al., Science 270:484-487, 1995). Tissue microarrays may also be used to evaluate reagents for cancer diagnostics, for instance specific antibodies or probes that react with certain tissues at different stages of cancer development, and to follow progression of genetic changes both in the same and in different cancer types, or in diseases other than cancer. Tissue microarrays may be used to identify and analyze prognostic markers or markers that predict therapy outcome for cancers. Tissue microarrays compiled from hundreds of cancers derived from patients with known outcomes permit one or more of DNA, RNA and protein assays to be performed on those arrays, to determine important prognostic markers, or markers predicting therapy outcome.

Tissue microarrays may also be used to help assess optimal therapy for particular patients showing particular tumor marker profiles. For example, an array of tumors may be analyzed to determine which amplify and/or overexpress HER-2, such that the tumor type (or more specifically the subject from whom the tumor was taken) would be a good candidate for anti-HER-2 Herceptin immunotherapy. In another application, tissue microarrays may be used to find novel targets for gene therapy. For example, cDNA hybridization patterns (such as on a DNA chip) may reveal differential gene regulation in a tumor of a particular tissue type (such as lung cancer), or a particular histological sub-type of the particular tumor (such as adenocarcinoma of the lung). Analysis of each at such gene candidates on a large tissue microarray containing hundreds of tumors would help determine which is the most promising target for developing diagnostic, prognostic or therapeutic approaches for cancer.

The methods and apparatuses disclosed herein provide a method for comparing image analysis systems or software in the interpretation of staining intensity or the type of histology or staining pattern. The methods and apparatuses disclosed herein provide a method method of comparing image analysis systems against one another to test, optimize and quality control the results. This approach could be used to optimize, develop and define clinical diagnostic kits for a large number of disease states.

The methods and apparatuses disclosed herein provide a method of testing automated tissue interpretation methods with manual methods (for example, a panel of experts who evaluate and diagnose a tissue specimen). The evaluation, assessment, or diagnosis of the automated method is compared with that arrived at by the manual method. The methods and apparatuses disclosed herein provide a method for training a computer-based system image analysis to recognize the same features on tissue microarrays as the human experts have scored. The methods and apparatuses disclosed herein provide a method for quality control of such automated tissue interpretation methods “machine vision” approaches between different models/approaches, from one day to another, calibrating with manual experts on a continuous basis, with different reagent systems in use, with different laboratory methods for the same target (such as different commercial kits), with different specimens orginating from the same or different laboratories, comparing the effects of other experimental procedures.

The methods and apparatuses disclosed herein provide a method of evaluating multiple samples from a neoplastic or nonneoplastic tissue to evaluate heterogeneity of a biomarker, to improve the sampling of different regions within a neoplastic or nonneoplastic tissue, to make results more comparabile with tissue microarray analysis of whole sections. Multiple samples from a specimen may be used to improve the reliability of the tissue microarray analysis, by providing an average biomarker content in a tissue specimen.

The methods and apparatuses disclosed herein provide a method of evaluating multiple samples from a primary tumor and its lymph node metastases, as well as distant metastases to compare differences in the biomolecule expression or genetic changes between the primary and metastatic specimens and in between metastatic specimens to identify and validate biomolecules that may predict metastatic progression or that may provide starting points for the development of treatment for metastatic cancer.

The methods and apparatuses disclosed herein provide a method of evaluating different regions in neoplastic or nonneoplastic tissue, based on histological type, grade, differentiation, degree of proliferation, invasion, atypia, angiogenesis, inflammation, necrosis, apoptosis, metastasis, tissue response to treatment, or other observable parameter or biological marker.

The methods and apparatuses disclosed herein provide a method of evaluating tumor areas defined by measurable properties, such as distance from the center of the tumor, periphery of the tumor, necrosis, inflammation, infection (such as viral) stromal boundary, normal epithelium boundary, border of invasion, or any other morphological feature.

The methods and apparatuses disclosed herein provide a method of evaluating regions within a tissue that comprise different cell types or histological structures, such as kidney glomeruli, collecting ducts, stroma etc.

The methods and apparatuses disclosed herein provide a method of evaluating regions within any type of tissue, such as atherosclerotic tissues with intimal thickening, early lesions, fully atheromas, thrombotic, complicted atheromas etc.

The methods and apparatuses disclosed herein provide a method of evaluating regions within an animal or plant species where one or more normal or abnormal organs or cell types are selected for arraying. This may include, for example, developmental stages within an animal or plant species, subregions within an organ, or any disease state affecting an organ or tissue.

The methods and apparatuses disclosed herein enable multi-parametric approaches for defining a combination of biomarkers that together have diagnostic or prognostic significance that is greater than any of the biomarkers alone. Because the high throughput nature of tissue microarray analysis, very large numbers of samples may be evaluated for very large numbers of markers, enabling the definition of sets of markers that may better define the biology of a particular disease state. For example, immunohistochemistry for a variety of tumor markers may be performed on tissue microarrays, followed by quantitative image analysis and statistical analysis. From this analysis, it may emerge that, for example, increased expression of three or four biomarkers is associated with a poor clinical prognosis, propensity to metastasize, or to respond or not respond to a particular type of therapy. Similar multiparametric approaches enable the definition of biomarkers that may predict progression in other diseases, allow disease subclassification, or provide help to diagnostic or therapy assesment. Similar multi-parametric approaches will be useful to study biological processes, such as cell differentiation, organ development and differentiation, proliferation and death.

The following additional examples illustrate how some particular assays would be performed with the automated system.

### EXAMPLE 1

Tissue Specimens

A total of 645 breast cancer specimens is used for construction of a breast cancer tumor tissue microarray. The samples include 372 fresh-frozen ethanol-fixed tumors, as well as 273 formalin-fixed breast cancers, normal tissues and fixation controls. The subset of frozen breast cancer samples is selected at random from the tumor bank of the Institute of Pathology, University of Basel, which includes more than 1500 frozen breast cancers obtained by surgical resections during 1986-1997. This subset is reviewed by a pathologist, who determines histological characteristics of the specimens. Other clinical information about the patients is also obtained (such as whether they have undergone chemotherapy, and what clinical stage of disease they had, as well as node status at the time of surgical resection). All previously unfixed tumors are fixed in cold ethanol at +4° C. overnight and then embedded in paraffin.

### EXAMPLE 2

Immunohistochemistry

After formation of the array and sectioning of the donor block, standard indirect immunoperoxidase procedures are used for immunohistochemistry (ABC-Elite, Vector Laboratories). Monoclonal antibodies from DAKO (Glostrup, Denmark) are used for detection of p53 (DO-7, mouse, 1:200), erbB-2 (c-erbB-2, rabbit, 1:4000), and estrogen receptor (ER ID5, mouse, 1:400). A microwave pretreatment is performed for p53 (30 minutes at 90° C.) and erbB-2 antigen (60 minutes at 90° C.) retrieval. Diaminobenzidine is used as a chromogen. Tumors with known positivity are used as positive controls. The primary antibody is omitted for negative controls. Tumors are considered positive for ER or p53 if an unequivocal nuclear positivity was seen in at least 10% of tumor cells. The erbB-2 staining is subjectively graded into 3 groups: negative (no staining), weakly positive (weak membranous positivity), strongly positive (strong membranous positivity).

### EXAMPLE 3

Fluorescent In Situ Hybridization (FISH)

Two-color FISH hybridizations are performed using Spectrum-Orange labeled cyclin D1, myc or erbB2 probes together with corresponding FITC labeled centromeric reference probes (Vysis). One-color FISH hybridizations are done with spectrum orange-labeled 20q13 minimal common region (Vysis, and see Tanner et al., Cancer Res. 54:4257-4260 (1994)), mybL2 and 17q23 probes (Barlund et al., Genes Chrom. Cancer 20:372-376 (1997)). Before hybridization, tumor array sections are deparaffinized at reagent station 110, air dried and dehydrated in 70, 85 and 100% ethanol followed by denaturation for 5 minutes at 74° C. in 70% formamide-2×SSC solution. The hybridization mixture includes 30 ng of each of the probes and 15 μg of human Cot1—DNA. After overnight hybridization at 37° C. in a humidified chamber, slides are washed and counterstained with 0.2 μM DAPI in an antifade solution. FISH signals are scored with double-band pass filters for simultaneous visualization of FITC and Spectrum Orange signals. Over 10 FISH signals per cell or tight clusters of signals are considered as indicative of gene amplification.

### EXAMPLE 4

mRNA In Situ Hybridization

For mRNA in situ hybridization, tumor array sections are deparaffinized and air dried before hybridization. Synthetic oligonucleotide probes directed against erbB2 mRNA (Genbank accession number X03363, nucleotides 350-396) are labeled at the 3′-end with ³³P-dATP using terminal deoxynucleotidyl transferase. Sections are hybridized in a humidified chamber at 42° C. for 18 hours with 1×10⁷ CPM/ml of the probe in 100 μL of hybridization mixture (50% forniarnide, 10% dextran sulfate, 1% sarkosyl, 0.02 M sodium phosphate, pH 7.0, 4×SSC, 1× Denhardt's solution and 10 mg/ml ssDNA). After hybridization, sections are washed several times in 1×SSC at 55° C. to remove unbound probe, and briefly dehydrated. Sections are exposed for three days to phosphorimager screens to visualize ERBB2 mRNA expression. Negative control sections are treated with RNase prior to hybridization, to abolish all hybridization signals.

The present method enables high throughput analysis of hundreds of specimens per array. This technology therefore provides a great increase in the number of specimens that can be analyzed, as compared to prior blocks where a few dozen individual formalin-fixed specimens are in a less defined or undefined configuration, and used for antibody testing. Further advantages of the present invention include negligible destruction of the original tissue blocks, and an optimized fixation protocol which expands the utility of this technique to visualization of DNA and RNA targets. The present method also permits improved procurement and distribution of human tumor tissues for research purposes. Entire archives of tens of thousands of existing formalin-fixed tissues from pathology laboratories can be placed in a few dozen high-density tissue microarrays to survey many kinds of tumor types, as well as different stages of tumor progression. The tumor array strategy also allows testing of dozens or even hundreds of potential prognostic or diagnostic molecular markers from the same set of tumors. Alternatively, the cylindrical tissue samples provide specimens that can be used to isolate DNA and RNA for molecular analysis.

### EXAMPLE 5

Novel Gene Targets

Tissue microarrays may be used to find, validate, prioritize and extend information on novel targets for cancer diagnostics or therapies. Hundreds of different genes may be differentially regulated in a given cancer (based on cDNA, e.g. microarray, hybridizations, or other high-throughput expression screening methods such as sequencing or SAGE). Similarly, proteomics techniques are available for detecting thousands of proteins in a cell. Combined with Internet database access to genomic sequence, functional genomics and proteomics databases, the future of biomedical research is based on analyzing thousands of parameters from each specimen. To date, there has not been a method available for high throughput tissue analysis using molecular pathological tools (such as mRNA ISH, IHC or FISH). Tissue microarrays enable the analysis of many sections sequentially (unlike the cDNA microarray concept, which allows multiple genes to be analyzed at once from a single specimen).

Analysis of each gene candidate on a large tissue microarray can help determine which is the most promising target for development of novel diagnostic methods, drugs, inhibitors, etc. For instance, a tumor microarray containing thousands of diverse tumor samples may be screened with a probe for an oncogene, or a gene coding for a novel signal transduction molecule, such as a G-proteinc coupled receptor. Such a probe may bind to one or a number of different tumor types. This can reveal a host of important information on the type of the molecular target. For example, it will give information on the presence or absence of the target in the tissue and cells therein; the quantity of the biomolecule in all the specimens; the distribution of the biomolecule with the various cell types in the various tissues, between cells in a tissue spot and variability between tissue spots. Tissue microarrays may also help to define the frequency of involvement of a particular biomarker in a large epidemiological sample, and it can provide information on critical clinico-pathological features of specimens expressing a particular biomarker. It can provide information on the difference between biomarker expression between normal and diseased tissues, or on the involvement of the biomarker during development and differentiation of tissues. This kind of information is important for addressing the relative importance of novel biomolecules as durg or diagnostic targets. The tissue microarray analysis will produc important information for investigators and companies in the field of genomics and proteomics on the clinical and biological significance of genes. At the same time, it will allow the diagnostic and pharmaceutical industry to find, validate, prioritize and optimize targets from the abundant genomic and proteomic information.

If a probe reveals that a particular gene is highly expressed and/or amplified in many tumors, then that gene may be an important target, playing a key role in many tumors of one histological type or in different tumor types. Therapies directed to interfering with the expression of that gene or with the function of the gene product may produce promising novel cancer drugs. In particular, the tissue microarrays can help to prioritize the selection of targets for drug development. Since there are thousands of candidate drug and diagnostic targets, such prioritization will greatly assist the search for novel therapies.

### EXAMPLE 6

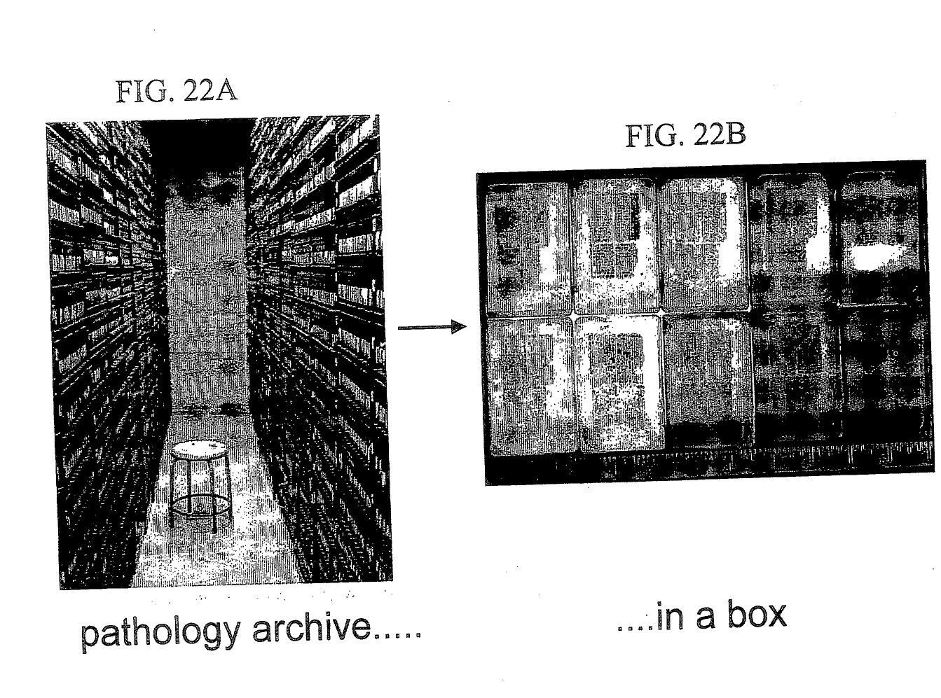

Uses of the Array (FIGS. 22-28)

FIGS. 22A and 22B illustrate that the arrays of the present invention can be used to greatly compress a pathological archive into a format that enables one to effectively carry out molecular analyses. In the past, such archives of individual tissue sections, from thousands of patients, mounted on slides, have occupied shelves of space in storage areas (FIG. 22A). This dramatically increases the utility of pathological archives in molecular analyses. Using the tissue microarrays disclosed herein, samples from thousands of tissue specimens can be arrayed on a single slide, as shown in FIG. 22B. Hundreds or thousands of copies of the array slides can be used to further increase the available information in the arrays. Before samples from the archive can be used for array constrcution, one needs to define the blocks and slides corresponding to a given patient, review the histology of the slides, select the right blocks and slides (often there are many per patient) for arraying, select and mark the regions of interest in these slides or blocks (either manually marking on the block surface or digitally with the specifications provided in this patent application), perform the arraying, perhaps generating multiple copies of the array blocks, section them on hundreds of slides, interrogate each with one or more reagents for a particular biomarker.

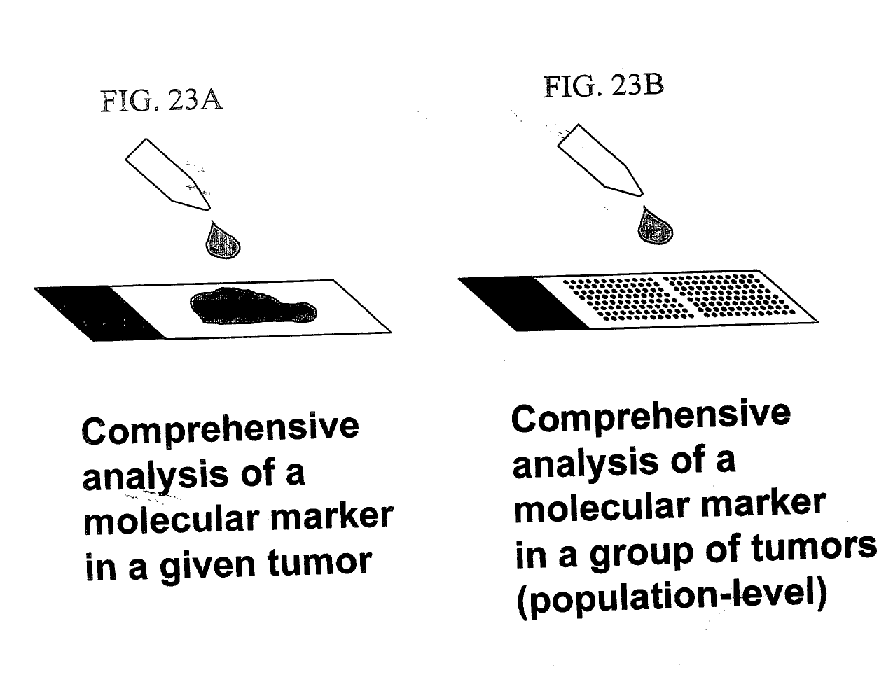

FIG. 23A illustrates the prior approach of exposing a single tumor section to a molecular marking agent (such as an IHC marker or a nucleic acid probe), to ascertain whether the agent recognizes a substrate of interest (such as a protein or DNA sequence). Use of the arrays shown in FIG. 23B, however, permits the simultaneous exposure of a molecular marker to a multiplicity of different tumors, under standardized conditions of array preparation and processing. The array therefore immediately provides an amount of information that would otherwise require laborious preparation of multiple tissue sections and processing steps (perhaps at multiple locations) which can introduce variability and scientific error into the analysis.

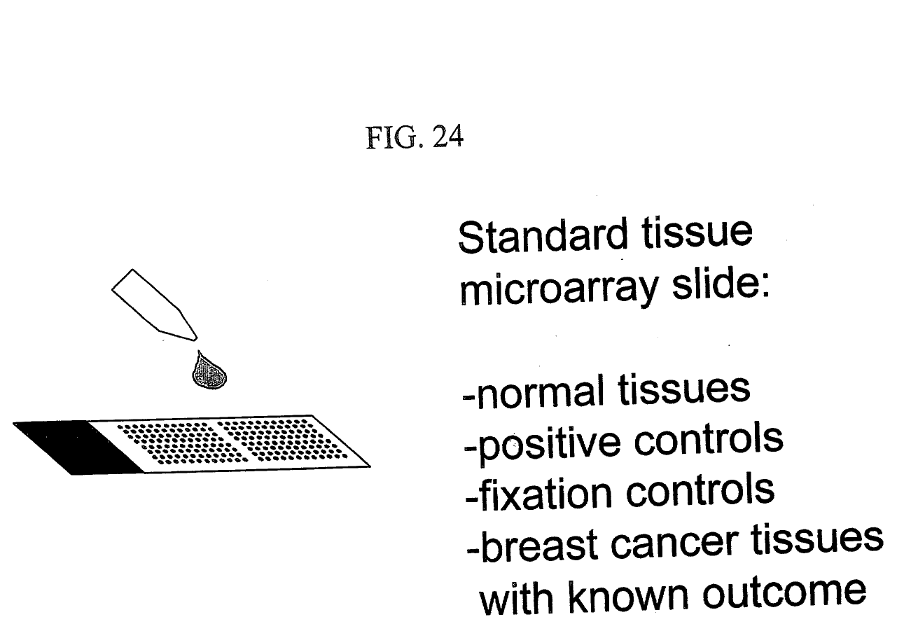

FIG. 24 illustrates that the array slides can be subjected to a bioanalysis at a single location, for example by a manufacturer of a test kit that contains an IHC marker such as a monoclonal antibody. The array can contain, for example, samples of normal tissues, positive controls, fixation controls, and/or tumors with known clinical outcomes, that have been exposed to the marker. For example, samples of the same tissue may be included that have been each fixed in a different fixative (such as formalin vs. ethanol), for various time-points and at various concentrations. Similarly, one can vary the time before fixation, to establish whether this pre-fixation delays causes variability in the biomarker detection from tissue microarrays or from conventional sections. Such tissue microarray slides may be used to evaluate how sensitive a particular staining reaction is to conditions used for fixing and treatment of the original tissue samples. For examples, some antigens may be very sensitive to the effects of fixation variations, while others can be very resistant. This kind of simple tissue microarray slides will provide important information to help developers of reagents, kits and other detection methods.

This slide (and corresponding array sections that are substantial copies of the slide) can then be sent to purchasers of the kit, who then possesses a compact and convenient reference to which the results of the purchasers' bioanalyses can be compared. Hence if the purchaser wants to determine if the tissue being analyzed is expressing a particular biomolecule, the purchaser reacts the tissue of interest with the IHC marker, and compares the result to the library of results on the array. Alternatively, the purchasers' results can be compared to standard results in the array, and those reactions that most closely match can be determined. If clinical outcomes are associated with a standard sample in the array, those clinical outcomes can be used to provide prognostic information about a patient having similar IHC results, or proposed treatments can be suggested by closely matching results.

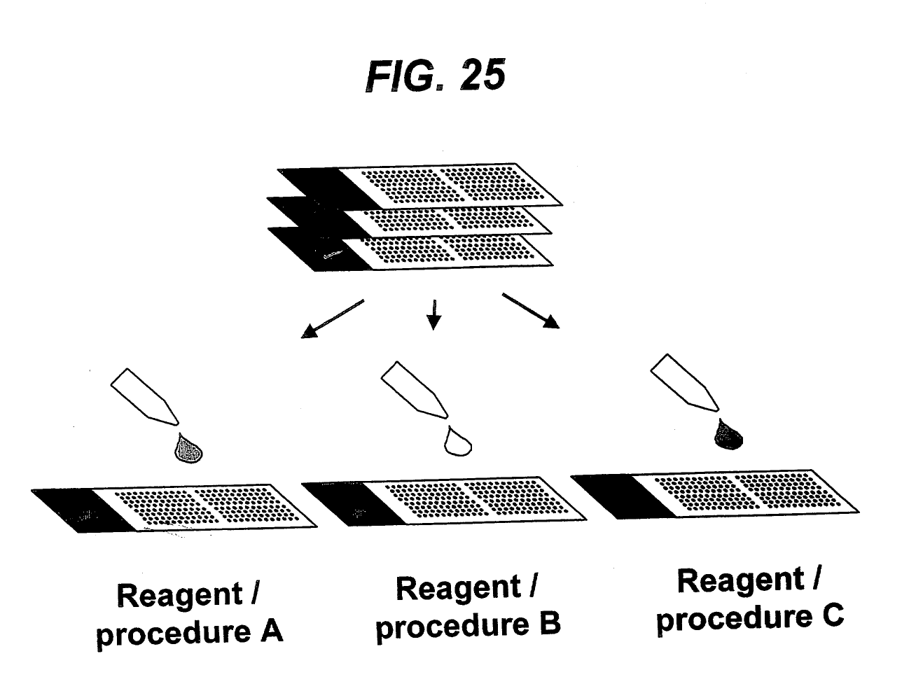

FIG. 25 illustrates a different use of the array, in which quality control of laboratory investigations of the biological material can be enhanced by obtaining multiple corresponding substantial copies of the array (for example by sectioning a block in which tissue cylinders have been placed), and then performing tests (for example with Reagent A, B or C by Procedure A, B or C) on the array copies. Since all of the samples on a single slide will be simultaneously exposed to Reagent A, variability of results (and consequent scientific error) will not be introduced by variations in Procedure A with the different samples. Similarly, all of the samples on a second slide will be simultaneously exposed to Reagent B, variability of results will not be introduced by variations in Procedure B. This allows effective testing and comparison of reagents, pretreatment methods, kits, staining conditions etc. on the same slide in otherwise identical conditions. This helps to determine the origins of variabilty, and to suggest measures that might reduce variability.

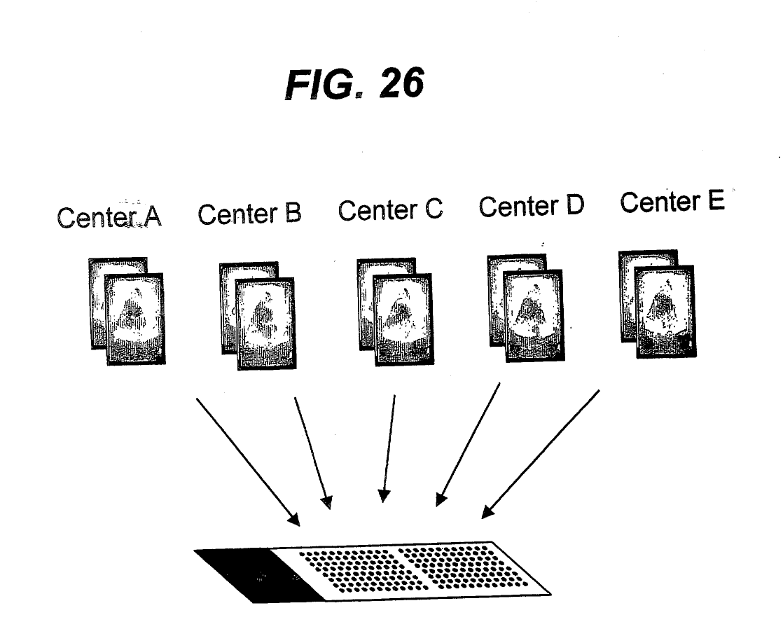

FIG. 26 illustrates how biological material from multi-center trials can be combined into a single array, and multiple copies of that array can be subjected to different biological analyses (not shown). Tissue specimens, for example surgical specimens of tumors, can be sent to a single location, where a sample is punched from each of the tumors and placed in a substrate, which is subsequently sectioned to obtain multiple corresponding sections, with corresponding samples at corresponding positions in the array. The multiple arrays are then subjected to different biological analyses.

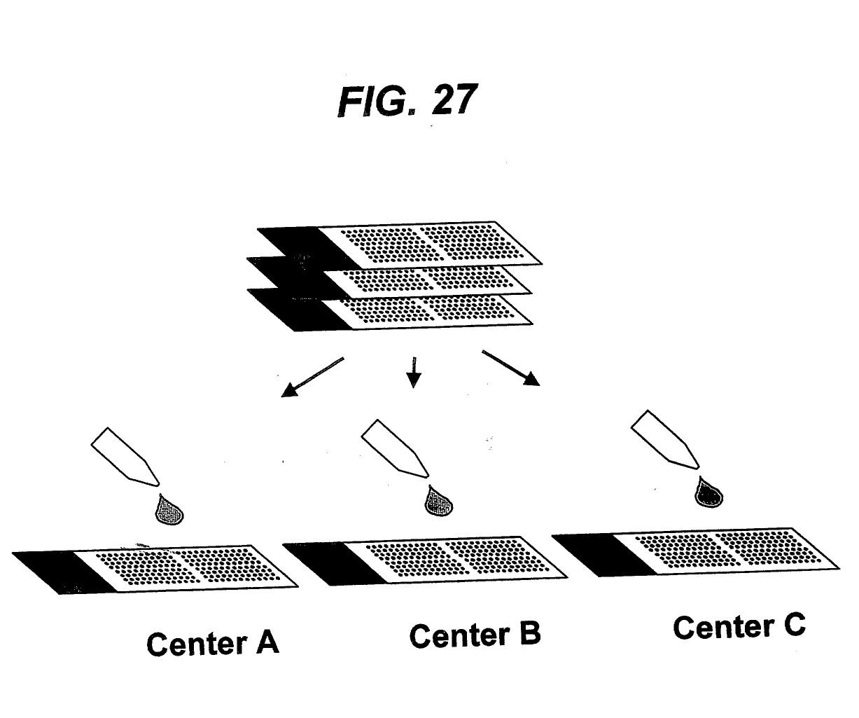

This approach allows several types of analyses. For example, it can be determined if a particular biomarker, test, kit etc. provides the same result from all kinds of samples fixed at different points in different institutes, then inserted to the same tissue microarray and used in the same experimental procedure. It could also be established whether similar results are obtained from samples from different institutions hat may have ethnic or demographic differences between patients, use different sampling strategies, fixation and other differences between one another). FIG. 27 illustrates that the multiple copies of the arrays can be used as a quality control device, to detect variations in reagents or procedures at different centers. For example, if a particulars IHC reagent is applied to different corresponding array sections at different centers (Centers A, B, C) the results of the procedures should be substantially identical. However, if the array sections from Centers A, B and C are subsequently examined and compared, differences in reactions (such as variability in positive IHC markers) can be attributed to variations in technique. Hence if the arrays treated at Centers A and C are substantially identical, but the array from Center B appears different, then quality control investigations can be undertaken with respect to the procedures used at Center B to stain the array.

Quality control can also be examined with respect to inter- or intra-observer bias. Hence the substantially identical arrays, which have been subjected to biological analyses (such as IHC staining or nucleic acid probing) at a single location may be distributed to different observers (such as collaborators at different institutions). Since the results of a test (such as Her-2 staining) should be essentially the same for each consecutive copy of the array, the different observers (A, B and C) can be asked to score or interpret the samples in the array. Alternatively, the esact same tissue microarray slide can be easily shipped from one location to another for analysis. An example of the score may be that the sample is Her-2 positive, Her-2 negative, or indeterminate. To the extent that an observer's scores (such as those of Observer B) differ from the scores of other observers (such as Observers A and C), the interpretations of Observer B can be discounted or discarded. Alternatively, information about the discrepancy can be provided to Observer B, so that Observer B can learn to conform his analyses to those of the other Observers. In this manner, greater uniformity of analysis is achieved. The observers may be observing digital images acquired from slides. Furthermore, one or more of the observers can be an imaging system/software that is being tested, teached, optimized or quality controlled to semi-automatically or automatically observe the staining characteristics of the slide.

A related problem with tissue examination is that it is often subject to variable interpretation by different examiners. Pathologic examination (including molecular analysis) is usually accomplished by microscopic examination of biological material by a clinician or researcher. When the clinician is a pathologist, important clinical decisions are often made based on an interpretation of the biological material. For example, if a bladder cancer specimen is judged to show a grade 3 (poorly differentiated) bladder tumor, the patient's bladder is often removed (cystectomy) because large scale studies have shown such surgery to be required to provide the greatest chance of survival. However, if the tissue is judged to show a grade 2 tumor (moderately differentiated) more conservative measures are adopted which would be inappropriate for more advanced disease. Since the selection of an appropriate treatment requires that pathologic diagnoses be made in accordance with uniform standards, methods are needed to help ensure that clinicians in different localities have uniform standards of histologic diagnosis.

Tissue microarrays may be used to address the problem of variable interpretation by different examiners. When the clinician is a pathologist, important clinical decisions are often made based on an interpretation of the biological material. For example, pathologists at different institutions (or even within the same institution) may differ on whether a particular bladder cancer is histologic grade 2, moderately differentiated, or histologic grade 3, poorly differentiated. The analysis carries profound implications for the patient: grade 2 tumors may be managed conservatively, whereas grade 3 tumors generally require radical cystectomy (bladder and lymph node removal). Similarly drastic decisions may be made depending on the interpretation of a particular immunostaining or other molecular marker. For ample, HERCEPTIN treatment is initiated for breast cancer, if the tumor is positive for the HER-2 gene/protein either by FISH analysis (for gene amplification) or by IHC (for protein overexpression). Similarly, estrogen receptor expression is gauged as a measure of the likelihood to get a response to hormonal therapy for breast cancer. Its is important to assure reproducibility and quantity control of such measurements in the cinical setting.

As an example, to address the variabiluty of tumor grading from one pathologist to another or between pathologists at different time points, tissue microarrays are constructed presenting several examples of various histologic grades of bladder cancer. Multiple substantial copies (for example sections mounted on microscope slides) of these tissue microarrays are disseminated to pathologists, trainees or other clinicians who interpret tissue histology. The dissemination may occur after the copies are reacted with biological reagents (such as hematoxylin-eosin staining or immunohistochemical staining) at a central site. Alternatively, multiple substantial copies may be disseminated to pathologists who perform reactions with biological reagents at a remote site. The substantial copies themselves may be disseminated, or images of the substantial copies may be disseminated.

A specimen of a suspected bladder cancer is obtained during a surgical procedure, and sent to a pathologist for diagnosis. The pathologist compares the tissue features and degree of differentiation in a surgical specimen with the features and degree of differentiation of the various bladder cancer in the tissue microarray. The pathologist may find that the degree of differentiation best matches the examples of grade 2 bladder carcinomas in the microarray. The pathologist then diagnoses grade 2, moderately differentiated bladder carcinoma. Alternatively, the pathologist examines the surgical specimen and arrives at a preliminary diagnosis of grade 2 bladder carcinoma. The pathologist then examines the tissue microarray to confirm the diagnosis, or revise the diagnosis to a different grade, such as grade 3 bladder carcinoma. In these and other manners, the use of tissue microarrays promote greater uniformity of diagnosis, and thereby improves therapy.

Although this example uses bladder carcinoma, the approach is readily adaptable to any other neoplastic or nonneoplastic disease in which tissue samples may be evaluated. For example, a microarray is constructed and disseminated having numerous examples of glomerulonephritis, and is used by a pathologist to assist the evaluation of a kidney biopsy. The microarray may be, for example, numerous examples of membranous glomerulonephritis, which may stained for light microscopic evaluation, or reacted with various immunological or immunohistochemical markers. The pathologist compares the light microscopic and immunologic features of the kidney biopsy to the various examples of nephritis contained in one or more tissue microarrays. The pathologist may use this comparison to conclude that the kidney biopsy represents an example or particular subtype of membranous glomerulonephritis.

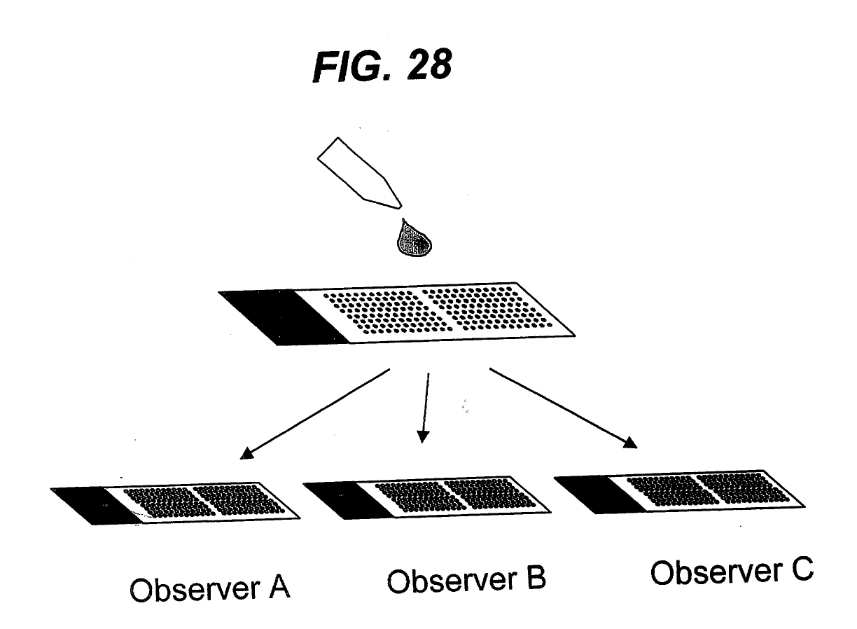

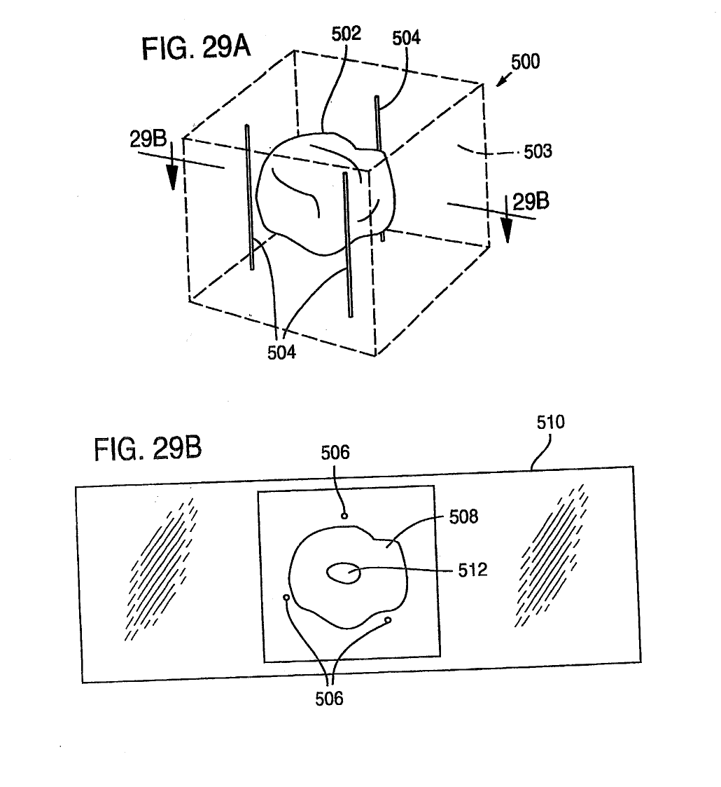

Alternatively, the different Observers A, B and C in FIG. 28 can be trainees, such as multiple medical students or pathology residents taking a qualifying examination. Each of the trainees has one of the array slides. The “correct” answers can be the analysis of each sample provided by an independent expert observer. Alternatively, “correct” answers can be obtained if Observers A, B and C are experts, and the multiple analyses can be used to help determine “correct” answers in situations in which an interpretation may be ambiguous. Moreover, many observers (such as at least 5, 10, 20, 50, 100 or more observers) can be asked to interpret the results of the bioanalysis, to provide an interpretation that has greater reliability (because inter-observer variability can be neutralized by the large number of observers). Such an approach can provide information analogous to that now obtained by multi-center meta-analysis of multiple studies. In this manner the biological significance of a molecular marker (such as Her-2) can be determined much more quickly, instead of requiring years of effort in different trials before a biologically reliable conclusion emerges.

Interpretation of molecular pathology results may become increasingly based on computer evaluation. Therefore, one or more of the observers can be a computer controlled imaging system that automaticlly or semi-automaticllys cores tissue samples for grade or staining intensity. Such results can be compared with the results of an expert, or a panel of experts, who have been asked to review the same slides. This multi-level assesment will make it possible to define optimal conditions, methods and quality control procedures for biomolecular detection in the clinical and research setting.

It is evident from the foregoing discussion that the arrays described herein can be used for a variety of purposes. In view of the many possible embodiments to which the principles of the invention may be applied, it should be recognized that the illustrated embodiments are examples of the invention, and should not be taken as a limitation on the scope of the invention. Rather, the scope of the invention is defined by the following claims. We therefore claim as our invention all that comes within the scope and spirit of these claims.

These and many other uses of the arrays, and examples of different bioanalyses that can be performed with the arrays, are disclosed in U.S. Provisional Application Nos. 60/106,038 and 60/075,979, and PCT publications WO9944063A2 and WO9944062A1, which have been incorporated by reference.

### EXAMPLE 7

Regions of Interest

A digital image representing a tissue donor block is acquired. A digital image enables the regions of interest (ROIs) on the block to be electronically stored for later use. The digital image may be acquired through several methods, and can include pre-annotation information. In one method, a pathologist or other examiner manually marks a microscope slide (containing a section from the tissue block) with a pen while viewing the tissue section under high magnification, and then acquires an image of the marked slide through any available means including a flatbed scanner. In another method, the examiner is supplied with a high-resolution image of a slide (containing a section from the tissue block) possibly acquired with a high-resolution camera mounted on a microscope capable of providing the needed resolution to define ROIs. In either case, the end result is a digital image of the slide that represents the tissue block. The difference, however, is that the first method yields an image that not only represents the block but also indicates the ROIs in the block.

Once the digital image is acquired, the marking information is represented electronically (digitally). The marking information consists of data points that can be later used to regenerate the perimeters of the ROIs.

Because no restrictions are placed, or guaranteed, on the slide image scaling or orientation relative to the block which it represents, additional information may be included to provide accurate marking information independent of the marked image scaling and orientation. This additional information can be provided by the use of indicia or reference points. In one embodiment, indicia or reference points are in approximately a same position in the original tissue block as they are in any slide representing the same tissue block. For example, if a slide were placed on top of the source tissue block in the appropriate orientation, the indicia or reference points of the block would align with those on the slide. One way of achieving such alignment is to embed the indicia or reference points in the block before sectioning, whether manually or automatically using a specialized apparatus, or tissue microarrayer, to embed the indicia or reference points. In this way the indicia or reference points would be sectioned along with the tissue block, and would maintain the approximately same position with respect to the tissue on the slide as they have on the tissue block the material embedded in the tissue blocks may be fluorescent, magnetic, or somehow distinctive from the surrounding tissue and block paraffin, to facilitate detection of the indicia or reference points in subsequent tissue microarray procedures.

Having three or more reference points in the block provides an added benefit if a reference point is lost, others would remain on the slide and they would be sufficient in generating the marking information. Representing the marking information as a function of the distance from reference points renders the ‘block marking’ process independent of rotations (i.e. when the slide image is a rotated version of the block). To make the process independent of scaling (i.e. when the slide image is physically larger or smaller than the block), information about the actual distances between the indicia or reference points themselves is included. Hence if reference points 1 and 2 for example are separated by 20 units on the slide and only 5 units on the block, the slide image size is four times that of the block. Thus the marking information consisting of ROI perimeters marked on the slide may be scaled appropriately.

Given a specific coordinate pair (x,y) the arrayer is capable of extracting the tissue sample located at (x,y) from the donor block and depositing it in a specified location in a recipient block. The arrayer has microarray constructor reference points, for example two or more microarray constructor reference point. These two microarray constructor reference points would have a known separation distance and known angle in reference to the arrayer coordinates. Additionally, the absolute coordinates of one of the two microarray constructor reference points is known. Furthermore, the microarray constructor reference points are in the field of view of the tissue microarrayer imaging system and appear on the same image as the tissue donor block.

A tissue donor block is provided with at least two embedded reference points and an image of the block itself, or of a slide made from a section of the block that retained at least two of the donor block reference points. As explained previously, this image may or may not have ROI markings.

A database including information on donor block ROIs may be constructed or maintained. The ROI points that define the perimeter are stored along with any other features that characterize this ROI (possibly including the type of cancer, punching priority, etc). When tissue from a ROI is extracted and deposited in a recipient block, enough information may be stored in the database to trace the recipient block tissue sample back to the originating donor block and vice versa The database may also store the different slide images that are associated with each block and possible updated block images that reflect extracted tissue.

Tissue block marking methods can be viewed as having two separate functions/stages. The first function provides a method for ROI marking of the slide image (or any other image representing the tissue block) and storing the information either in a standalone file or a database. The second function provides a method for regenerating the ROI perimeters using the stored information from the first function/stage, and generating tissue microarrayer punch locations within the ROI. Each function/stage can be enhanced to provide the user more options and flexibility.

Marking Stage

In the marking stage, the image indicia or reference points are assigned in a predefined order that would be compatible with later use of the same reference points on the block at the time of regeneration of ROIs on the tissue microarrayer. After locating and selecting the reference points, a method for marking of ROIs is provided. In the case of the ROIs being previously marked on a slide with a pen before the image was acquired, the user's responsibility is reduced to retracing over those pen lines defining the ROI. In the case of no previous markings, the user must identify the ROIs on the image and mark them directly. Tools to mark polygons, circles, or scattered points on the image are readily available. Furthermore, the user can mark a subregion of a specific ROI to exclude from the full ROI. The user can annotate the ROIs with their tissue type or other needed properties, characteristics, or instructions. These annotations can be automatically associated/linked with the recipient tissue microarray block to which this tissue is transferred to identify the arrayed tissue. Additionally, properties of the block itself can also be stored for later use.

If scaling information is known, then additional features can be implemented. One method to incorporate scaling information is to include two points of known physical separation on the slide before an image of the slide is acquired. A user can locate these two points on the slide image, which will allow precise calculation of the scaling information.

This scaling information can be used in the tissue microarraying process, for example to calculate an estimate of the amount of tissue available in each ROI. The number of punches which can be extracted from each ROI defined within the block can then be computed. Furthermore the amount of tissue having certain characteristics can be calculated per block, not just per ROI. There are many benefits to having this information. As an example, a pathologist searching for a specific type of tissue could easily query the database containing the block marking information, and retrieve blocks that would provide the required quantity of tissue meeting the specific tissue criteria.

Scaling information would also enable tissue microarrayer punch locations to be generated and stored, and downloaded later for actual tissue extraction. This would provide the pathologist more control over punch locations. The pathologist may manually manipulate (fine tune) the locations of the automatically generated punch locations, delete certain punches, mark certain areas as not desired, assign specific punches to certain recipient blocks, etc. In summary, the pathologist could then more easily control the extraction process down to the individual punches.

ROI Regeneration Stage

Once the indicia or reference points are located, the marking information can be retrieved to regenerate the ROI information previously saved at the time of marking the image representing the block. The distance between the reference points on the block image is compared to the distance between the reference points on the slide image (which was saved along with ROI information). This comparison yields a scaling factor that can be used to scale all the ROI saved information and display it in the correct scale on the block image. At this point, the user may manually mark the microarray constructor reference points, or allow the to be automatically detected. The conveniences of being able to move punch locations around, delete certain punches, mark certain punches as undesired, add punches, etc. are also provided to the user at this stage.

Changes that occur to the tissue block at this stage due to tissue extraction may be stored in the database. Consequently, if the tissue at a certain location was extracted and deposited in a recipient block, then information about the position of extraction is stored in the database to prevent a user from attempting to mark the same position for extraction at a later point. Along with the fact that the tissue was extracted at the given position, the recipient block in which that specific punch was deposited has its unique identification stored with the donor block information so as to be able to trace it down given the donor block information. In a similar way, the donor block identification is stored with the recipient block data so as to be able to identify which donor block contributed to which tissue sample in the recipient block. The arrayer control station may relay information regarding which punch location from the donor block was extracted and in which recipient block it was deposited so that changes may be stored in the database. Alternatively, if the arrayer is connected to the database, it can directly commit the changes.

Scaling information would allow for recipient block design. A pathologist wishing to compose an array of possibly different tissue types can proceed through the following steps:

1) Layout the arrangement of the array, or possibly subarrays, within the recipient block, i.e. a 4×6 subarray of tissue type a, 5×5 subarray of tissue type b, etc.

2) Specify the punching properties for both the donor and recipient block (i.e. specify the punch size for each subarray, punch spacing for both the donor and recipient, etc.)

3) Submit the array request to the database.

At this point, exact quantities of each specific tissue type requested by the user are calculated. A query is formed to the database to return a list of the blocks that can satisfy the request along with information on where to extract and how much to extract from each individual returned block. These blocks, along with the information on where to punch from each, can then be used for actual tissue extraction, and formation of the desired recipient array. Once tissue is extracted, the database is updated to include information on which recipient array, and where within the recipient array, the tissue was deposited. Also, the recipient array information would be stored in the database, and would include which donor block the tissue originated. Adding this information to the database allows a user who is viewing a specific donor block's information to determine the recipient array in which the extracted tissue from the donor was deposited, and a user viewing the recipient array can trace each tissue sample back to the source donor block.

Since database queries can be submitted remotely, a pathologist or other user does not need to be physically near the tissue microarray process and instrumentation. The tissue microarraying operation can be entirely completed without the pathologist.

In view of the many possible embodiments to which the principles of the invention may be applied, it should be recognized that the illustrated embodiments are examples of the invention, and should not be taken as a limitation on the scope of the invention. Rather, the scope of the invention is defined by the following claims. We therefore claim as our invention all that comes within the scope and spirit of these claims.

We claim:

1. An automated apparatus for preparing tissue specimens for analysis, comprising:
a specimen source from which specimens are retrieved from assigned locations;
a retriever that retrieves the specimens from the specimen source; and
a constructor that removes tissue samples from a plurality of the specimens, and arrays the samples at identifiable locations in three dimensional arrays in a plurality of substrates, wherein at least some of the different identifiable locations correspond to samples from different specimens; and
a controller that directs the retriever and constructor.

2. The automated apparatus of claim 1, further comprising a sectioner that sections the three dimensional arrays into cut sections which carry the samples from different specimens, wherein the locations in the three dimensional arrays correspond to locations in the cut sections.

3. The automated apparatus of claim 2, wherein the controller further directs the sectioner.

4. The automated apparatus of claim 1, wherein the controller further comprises a recorder that records an identification of a subject associated with a particular specimen, and the identifiable locations in the three dimensional arrays and the cut sections.

5. The automated apparatus of claim 4, further comprising a scanner that detects a position of the specimens, to determine locations from which the controller directs ambles to be taken.

6. The automated apparatus of claim 4, further comprising an automated biomarker station wherein the cut sections undergo one or more labeling reactions that identify biological substrates in the cut sections.

7. The automated apparatus of claim 6, further comprising an automated image analyzer that captures images of the cut sections, and detects a presence of biomarkers in samples in the cut sections.

8. The automated apparatus of claim 1, wherein the specimen source comprises a plurality of specimens at assigned locations.

9. The automated apparatus of claim 8, wherein the specimens are embedded in blocks of embedding medium, and the blocks are carried by carriers that are storable in the specimen source.

10. The automated apparatus of claim 9, wherein the carriers carry identifiers that are recognizable by the controller.

11. The automated apparatus of claim 10, wherein the specimen source comprises recipient stations in which the carriers are received.

12. The automated apparatus of claim 1, further comprising an automated locator that locates a region of interest in the specimen source.

13. The automated apparatus of claim 12, further comprising providing a reference indicium which extends at least partially through the specimen source.

14. The automated apparatus of claim 13, wherein the reference indicium comprises an elongated marker that extends at least partially through the specimen source.

15. The automated apparatus of claim 14, wherein the specimen source comprises substantially parallel top and bottom surfaces, and the indicium extends substantially perpendicular to the top and bottom surfaces.

16. The automated apparatus of claim 13, wherein the reference indicium comprises a plurality of separate reference indicia.

17. The automated apparatus of claim 16, wherein the plurality of reference indicia are elongated and substantially parallel to one another.

18. The automated apparatus of claim 17, wherein the specimen source comprises substantially parallel top and bottom surfaces, and the elongated indicia extend substantially perpendicular to the top and bottom surfaces.

19. The automated apparatus of claim 13, wherein a region of interest is located by measuring a distance from the reference indicium.

20. An apparatus for constructing tissue arrays from a plurality of donor tissue specimens, comprising:
a donor source containing a plurality of identifiable donor tissue specimens;
a retriever that retrieves the donor tissue specimens from the donor source;
a tissue array constructor receiving donor tissue specimens retrieved by the retriever, the tissue array constructor obtaining tissue samples from different tissue specimens retrieved by the retriever and inserting the tissue samples into recipient blocks, thereby constructing a tissue array; and
a controller operating the retriever and array constructor, the controller further identifying tissue samples within the array.

21. Thy apparatus of claim 20, wherein the tissue sample is obtained from a region of interest in the tissue specimen.

22. The apparatus of claim 21, wherein the tissue sample is used in cell free analysis.

23. The apparatus of claim 22, wherein the cell free analysis is an analysis of a biomolecule obtained from the tissue sample.

24. The apparatus of claim 23, wherein the biomolecule is selected from the group consisting of genomic DNA, partial genomic DNA, mRNA, cDNA, and polypeptide.

25. The apparatus of claim 23, wherein the cell free analysis is a method of detecting a mutation in the tissue sample.

26. The apparatus of claim 22, wherein the cell free analysis is selected from the group consisting of DNA sequencing, restriction fragment length polymorphism determination, Southern blotting or other forms of DNA hybridization analysis, determination of single-strand conformational polymorphisms, comparative genomic hybidization, mobility-shift DNA binding assays, protein gel electrophoresis, Northern blotting and other forms of RNA hybridization analysis, protein purification, chromatography, immunoprecipitation, protein sequence determination, Western blotting (protein immunoblotting), ELISA or other forms of anybody-based protein detection, isolation of biomolecules for use as antigens to produce antibodies, PCR, RT PCR, differential display, serial analysis of gene expression, and protein truncation test.

27. The apparatus of claim 20, wherein the controller identifies tissue samples by recognizing identifiers associated with the tissue specimens, wherein the retriever recognizes the identifiers and retrieves a specified tissue specimen from the donor source.

28. The apparatus of claim 20, wherein the tissue specimens are associated with a carrier medium, and the apparatus further comprises a locator that records a location of the tissue specimen in the carrier medium.

29. The apparatus of claim 22, wherein the carrier medium comprises a tissue block Medium in which the tissue specimens are embedded.

30. The apparatus of claim 29, wherein the locator also marks the tissue block medium with an identifier that identifies the tissue specimen within the tissue block medium.

31. The apparatus of claim 20, wherein the donor source comprises tissue specimens positioned in a donor specimen storage station, from which the constructor obtains tissue samples for insertion into the array.

32. The apparatus of claim 31, wherein the retriever further returns the tissue specimens to the storage station after obtaining tissue samples for insertion into the array.

33. The apparatus of claim 20, wherein the retriever further comprises a coordinate positioning device that positions the retriever for retrieval of a particular tissue specimen from the donor source.

34. The apparatus of claim 20, wherein the retriever further comprises a robotic arm that retrieves tissue specimens from the donor source, transfers tissue specimens to the tissue array constructor, and returns tissue specimens to the donor source.

35. The apparatus of claim 20, wherein the tissue array constructor comprises:
a holder that can be positioned to hold a tissue specimen and a recipient block having an array of receptacles; and
a reciprocal punch positioned in relation to the holder to punch a tissue sample from the tissue specimen, and deliver the tissue specimen to an identifiable receptacle in the recipient block.

36. The apparatus of claim 35, wherein the holder comprises a coordinate positioning device that can be incrementally positioned to align a predetermined receptacle with the reciprocal punch.

37. The apparatus of claim 34, further comprising a recorder for recording a position of the receptacle in the recipient block, and an identity of the tissue specimen placed in the receptacle.

38. The apparatus of claim 20, further comprising a microscope for locating a structure of interest in a reference slide aligned with the tissue specimen.

39. The apparatus of claim 20, further comprising a recipient block source containing a plurality of recipient blocks, each recipient block including a plurality of tissue samples, the location of the recipient block being identifiable in the recipient block source.

40. The apparatus of claim 39, wherein the recipient block source is an array of recipient blocks.

41. The apparatus of claim 39, wherein the donor source and the recipient block source are a single station.

42. The apparatus of claim 39, wherein the retriever further returns tissue donor specimen's to the recipient block source after constructing the tissue array.

43. The apparatus of claim 20, further comprising a recipient block sectioner, which cuts sections from the recipient block into a plurality of cut sections.

44. The apparatus of claim 43, wherein the sectioner further mounts cut sections on a solid support.

45. The apparatus of claim 43, further comprising a processing station that exposes the cut sections to reagents that recognize biological structures in the cut sections.

46. The apparatus of claim 43, further comprising an imager that obtains an image of cut sections.

47. The apparatus of claim 46, wherein the imager further comprises an image processor that identifies regions of the cut sections that contain images of biological interest.

48. The apparatus of claim 46, wherein the cut sections contain biological markers, and the imager further comprises:
a detector that detects one or more biological markers present in the cut sections; and
a storage device which stores images of the cut sections.

49. The apparatus of claim 46, wherein the detector further comprises a quantifer that quantifies a quantity of the biological marker in the cut sections.

50. The apparatus of claim 46, wherein the detector further comprises a locator that determines a distribution of the biological marker in the cut sections.

51. The apparatus of claim 43, further comprising a database containing identifying information about the cut sections and the subjects from which the tissue specimens were obtained.

52. The apparatus of claim of 51, wherein the database further includes a quantity and a distribution of at least one biological marker in tissue array sections.

53. The apparatus of claim 52, wherein the database further includes:
a location of tissue donor specimens;
an identity and location of the tissue samples in the tissue array; and
an identity and location of recipient blocks in the recipient block array.

54. An apparatus for assembling tissue arrays, comprising:
a donor specimen station which includes compartments for assigned tissue specimens;
a computer readable identifier which identifies the tissue specimens in the donor specimen station;
a donor block scanner for determining a location of the tissue specimens in the carrier;
a tissue array fabricator for obtaining a plurality of elongated tissue samples from a plurality of tissue specimens, and placing the plurality of elongated tissue samples in a recipient block;
a sectioner that sections the recipient block sufficiently transverse to the elongated tissue samples to form a series of block sections which retain a relationship of the elongated tissue samples in the recipient block;
a processing station that exposes different sections to different biological markers that associate with biological substrates of interest in the sections, if the biological substrates are present; and
a scanner that scans the different sections to detect the presence of the biomarkers in the different sections.

55. The apparatus of claim 54, further comprising a controller that automatically identifies the tissue specimens in the carrier, obtains a plurality of elongated tissue samples, places the plurality of elongated samples in the recipient block, sections the recipient block, exposes the different sections to the different biological markers, and detects the presence of the biomarkers.

56. The apparatus of claim 54, further comprising one or more of:
the computer readable identifier comprises a computer readable label associated with the tissue specimens;
the elongated tissue specimens are embedded substantially parallel to one another in embedding medium, and the tissue fabricator sections the embedding medium substantially transverse to the tissue specimens;
the processing station exposes different sections to biological markers that include one or more of histological stains and markers that hybridize with nucleic acids.

57. The apparatus of claim 55, further comprising one or more robotic transporters that move specimens or sections between the donor specimen station, the donor block scanner, the tissue array fabricator, the sectioner, the processing station, and the scanner.

58. The apparatus of claim 54, further comprising a database that includes information about a subject from whom the tissue specimens were obtained, and is capable of correlating that information with the presence of the biomarkers in the different sections.

59. A device for performing molecular analysis of biological specimens, comprising:
storage means for storing a plurality of biological specimens embedded in embedding medium;
automated array forming means for obtaining multiple tissue samples from a biological specimen, inserting the tissue samples in corresponding positions in different recipient substrates to make multiple arrays of similar biological specimens, and sectioning the recipient substrates to make multiple corresponding sections of each recipient substrate;
automated reaction means for reacting the multiple corresponding sections of the different recipient substrates with biological reagents that react with biological substrates of interest in the sections;
automated detection means for detecting a presence, or a quantity, or both a presence and a quantity, of the biological reagent in the sections; and
computer means for recording information about subjects from whom the biological specimens were obtained, and correlating that information with the presence, quantity, or presence and quantity of the biological reagent in the sections.

60. A method for performing molecular analysis of biological specimens, comprising:
providing multiple sections each comprising multiple biological samples;
exposing different sections to different biological reagents that react with biological markers in the biological samples
obtaining images of the different sections after exposing the sections to the different biological reagents; and
analyzing the images to determine whether a reaction with a biological marker has occurred in the different specimens.

61. The method of claim 60, further comprising exposing a section or sections to multiple biological reagents, to detect a plurality of biological markers in the section.

62. The method of claim 61, wherein the molecular analysis is an analysis of tissue, cellular, or subcellular distribution of the biological marker.

63. The method of claim 62, wherein the multiple biological samples were obtained from multiple different biological specimens.

64. The method of claim 60, wherein the multiple biological specimens were obtained from different subjects.

65. The method of claim 60, further comprising obtaining information about subjects from whom the biological specimens were obtained, and associating that information with the results of analyzing the images to obtain relationships between the information and the reaction.

67. The method of claim 61, wherein the biological samples are samples from tissue specimens, and there are at least 20 different tissue specimens present in each different section.

68. The method of claim 67, wherein the different tissue specimens are exposed to at least 20 different reagents.

69. The method of claim 63, wherein the biological samples are samples from at least 100 different tissue specimens in each different section.

70. The method of claim 61, wherein the different tissue specimens are exposed to at least 100 different reagents.

71. The method of claim 60, wherein obtaining images comprises obtaining digital images and storing the digital images.

72. The method of claim 61, wherein analyzing the images further comprises quantifying the reaction with the biological marker.

73. The method of claim 60, wherein providing the plurality of sections comprises obtaining multiple elongated biological samples from multiple biological specimens, fixing the elongated biological samples substantially parallel to one another in a substrate, and sectioning the substrate substantially transverse to the elongated biological samples.

74. The method of claim 73, wherein obtaining the multiple elongated biological samples comprises retrieving tissue specimens from a donor block array of tissue donor blocks, wherein the tissue specimens are marked with computer readable identifiers.

75. A method for performing molecular analysis of biological specimens, comprising:
obtaining with a tissue microarray constructor a plurality of samples from regions of interest one or more tissue samples;
performing one or more cell free analyses to observe one or more biological markers in the tissue samples.

76. The method of claim 75, wherein the cell free analysis is an analysis of a biomolecule obtained from the tissue sample

77. The method of claim 76, wherein the biomolecule is selected from the group consisting of genomic DNA, partial genomic DNA, mRNA, cDNA, and polypeptide.

78. The method of claim 76, wherein the cell free analysis is a method of detecting a mutation in the tissue sample.

79. The method of claim 76, wherein the cell free analysis is selected from the group consisting of DNA sequencing, restriction fragment length polymorphism determination, Southern blotting or other forms of DNA hybridization analysis, determination of single-strand conformational polymorphisms, comparative genomic hybidization, mobility-shift DNA binding assays, protein gel electrophoresis, Northern blotting and other forms of RNA hybridization analysis, protein purification, chromatography, immunoprecipitation, protein sequence determination, Western blotting (protein immunoblotting), ELISA or other forms of anybody-based protein detection, isolation of biomolecules for use as antigens to produce antibodies, PCR, RT PCR, differential display, serial analysis of gene expression, and protein truncation test.

80. A method for constructing tissue microarrays from a plurality of donor specimens, comprising:
providing a donor array of tissue donor blocks, each block including a tissue specimen embedded in embedding medium and being identifiable in a donor block array;
retrieving identified tissue donor blocks from the donor block array;
obtaining tissue samples from retrieved tissue donor blocks and inserting tissue samples from the tissue specimen into different recipient blocks; and
sectioning the blocks.

81. The method of claim 80, further comprising:
determining coordinates of the tissue specimen within the donor block; and
storing the coordinates.

82. The method of claim 80, further comprising:
determining coordinates of a region of interest, and storing the coordinates.

83. The method of claim 82, further comprising storing annotations associated with the region of interest.

84. The method of claim 82, wherein the tissue sample is obtained from a region of interest.

85. The method of claim 83, wherein multiple tissue samples are obtained from multiple regions of interest.

86. The method of claim 81, further comprising:
marking the tissue donor block with a computer readable indicator identifying a source of the tissue specimen, and the coordinates.

87. The method of claim 82, further comprising punching a plurality of receptacles in a recipient block;
punching tissue specimen samples from the donor blocks, and placing tissue specimen samples in receptacles in the recipient block.

88. The method of claim 87, wherein punching the receptacle in the recipient block and punching the tissue specimen sample from the donor block are performed by two or more different punches.

89. The method of claim 88, wherein punching tissue specimens from the donor block comprises placing the donor block in a holder below the reciprocal punch, and advancing it to a region of interest.

90. The method of claim 89, wherein the region of interest is determined by reference to the stored coordinates of the tissue specimen contained within the donor block.

91. The method of claim 89, wherein a plurality of tissue specimens are obtained from a plurality of regions of interest.

92. The method of claim 90, wherein the region of interest is determined by examining a thin section cut from the donor block.

93. The method of claim 80, wherein a plurality of recipient blocks are stored in a recipient block array.

94. The method of claim 80, further comprising storing recipient block tissue identity information identifying tissue specimens contained within receptacles in recipient blocks, and recipient block location information defining the recipient block in a recipient block array.

95. The method of claim 81, further comprising marking recipient blocks with recipient block tissue identity information and recipient block location information.

96. The method of claim 87, further comprising:
retrieving a recipient block from the recipient block array;
positioning the recipient block on a sectioning device;
cutting sections from the recipient block to form cut sections; and
mounting the cut sections on a solid support, thereby generating tissue microarray sections.

97. The method of claim 96, wherein a recipient block retriever transfers the recipient block from the recipient block array to the sectioning device, and returns it to the recipient block array.

98. The method of claim 97, further comprising marking the solid supports with information identifying the tissue specimens in the tissue microarray sections mounted thereon.

99. The method of claim 98, further comprising placing the tissue microarray sections into position for treatment with one or more reagents, and treating the tissue microarray sections with one or more reagents.

100. The method of claim 96, further comprising analyzing the tissue microarray sections for the presence of biological markers.

101. The method of claim 100, wherein the method of analyzing tissue microarray sections further comprises:
(a) placing a tissue microarray section into position for analysis;
(b) analyzing the tissue microarray section for the presence of biological markers;
(c) moving the tissue microarray section out of the position for analysis;
(d) placing a different tissue microarray section into position for analysis;
(e) repeating steps (b)-(d) a plurality of times.

102. The method of claim 101, wherein the analysis for biological markers further comprises:
obtaining an image of a tissue microarray section;
processing the image to identify specific regions that correspond to the presence of a biological marker;
determining the amount and distribution of the biological marker that is present in the tissue microarray section;
storing the image obtained; and
storing information regarding the amount and distribution of biological marker present in the tissue microarray section.

103. A computer implemented system for rapid construction and analysis of tissue microarray sections, comprising:
a recipient block retriever obtaining recipient blocks from a recipient block array, and transferring recipient blocks to a sectioner;
the sectioner cutting sections from recipient blocks, and mounting the sections on a solid support;
a conveyor transferring the mounted sections to a processor;
the processor processing the mounted samples for biological analysis;
an image analyzer, imaging tissue microarray sections and analyzing them for presence of biological markers;
a database, storing information identifying tissue samples analyzed, and information obtained from analysis of tissue microarray sections for presence of biological markers.

104. The computer implemented system of claim 103, wherein information stored in the database further comprises annotations.

105. The computer implemented system of claim 103, wherein the database comprises information regarding quantity or distribution of biological markers in a tissue microarray section.

106. The computer implemented system of claim 103, wherein the database comprises information regarding subcellular distribution of biological markers in a tissue microarray section.

107. The computer implemented system of claim 103, wherein information obtained from the analysis of biomarkers in the tissue microarray sections is correlated with an annotation in the database.

108. The computer implemented system of claim 107, wherein the annotations comprise information relating to the subject from whom the tissue sample was obtained.

109. The system of claim 103, wherein the system further comprises:
a plurality of different stations for the sectioner, processor and image analyzer;
a conveyor transporting mounted samples between stations;
a plurality of robotic arms that expose the mounted sections to biological reagents for biological analysis; and
a controller controlling the transport of mounted sections to stations, the time that samples remain at individual stations, and the amount of time that sections are exposed to biological reagents.

110. A method of examining a biological sample, comprising:
providing a plurality of biological samples at identifiable positions in an array;
subjecting the biological samples in the array to a biological analysis;
examining the array to detect a biological marker;
wherein the biological analyses are performed or analyzed at multiple different locations.

111. The method of claim 110, comprising subjecting multiple substantial copies of the array to a same biological analysis.

112. The method of claim 110 wherein the biological analysis is an analysis with a specific binding agent.

113. The method of claim 112, wherein the specific binding agent comprises an antibody or a nucleic acid.

114. The method of claim 113, wherein the specific binding agent comprises a nucleic acid probe.

115. The method of claim 110, wherein the multiple substantial copies of the array are obtained by providing elongated samples at identifiable locations in a substrate, and sectioning the substrate.

116. The method of claim 115, wherein the elongated samples are substantially parallel, and the substrate is sectioned transverse to the samples.

117. The method of claim 110, wherein at least one of the multiple substantial copies is subjected to a reference biological analysis, and multiple substantial copies are disseminated to one or more observers to subject the copies to the same biological analysis.

118. The method of claim 117 wherein the one or more observers compare the results of the same biological analysis to the reference biological analysis.

119. The method of claim 118, wherein the one or more observers comprises:
(a) different researchers;
(b) trainees who are learning to perform the biological or pathological analysis; or
(c) an automated image analysis system

120. The method of claim 119, wherein the one or more observers are the different researchers, who compare a result of their biological analysis to the reference biological analysis.

121. The method of claim 120, wherein an interpretation of the biological analysis of the different researchers is compared to an interpretation of the reference biological analysis to perform quality control.

122. The method of claim 120, wherein the biological analysis of the different researchers is compared to the reference biological analysis to determine whether a reagent used by the different researchers performs comparably to a reagent used in the reference biological analysis.

123. The method of claim 122, wherein the reagent is an immunohistochecmical or nucleic acid marker.

124. The method of claim 119, wherein the one or more observers comprises the trainees, and the results of the biological analysis of the trainees is compared to the reference biological analysis.

125. The method of claim 124, wherein the trainees indicate a proposed interpretation of the biological analysis, and the proposed interpretation is compared to a reference interpretation of the reference biological analysis.

126. The method of claim 125, wherein the trainees are test takers, who are graded by comparing the proposed interpretation to the reference interpretation.

127. The method of claim 117, wherein the reference interpretation is obtained by combining an interpretation of multiple observers.

128. The method of claim 110, wherein the array which has been subjected to the biological analysis is disseminated to multiple observers.

129. The method of claim 128, wherein the multiple observers are at multiple locations.

130. The method of claim 129, wherein the array is disseminated to the multiple observers in electronic form.

131. The method of claim 130, wherein the electronic form is via a communication channel or a computer readable medium.

132. The method of claim 131, wherein the communication channel is a global communication system.

133. The method of claim 131, wherein the computer readable medium is a CD-ROM, a CD-R, a CD-RW, a DVD, or an optical disc.

134. The method of claim 110, wherein the array is a microarray.

135. The method of claim 110, wherein the plurality of biological samples comprises at least 100 biological samples.

136. The method of claim 135, wherein the plurality of biological samples comprises at least 500 biological samples.

137. The method of claim 135, wherein the plurality of biological samples comprises at least 1000 biological samples.

138. The method of claim 110, wherein the identifiable positions comprise coordinates of the array.

139. The method of claim 138, wherein the array comprises a substantially uniform matrix of rows and columns.

140. The method of claim 138, wherein the biological samples comprise samples of tissue specimens.

141. The method of claim 140, wherein the tissue specimens comprise pathology specimens.

142. The method of claim 140, wherein the tissue specimens comprise one or more of:
(a) neoplastic tissue;
(b) non-neoplastic tissue;
(c) a combination of neoplastic and non-neoplastic tissue; or
(d) comparative specimens of different stages in a biological spectrum.

143. The method of claim 142, wherein the comparative specimens comprise one or more of:
(a) different stages in development of a tumor;
(b) different types of tumor;
(c) different stages in progression of a biologically dynamic tissue;
(d) multiple samples from the same tissue specimen or region of interest; or
(e) specimens of a tumor and specimens of a metastasis of that tumor.

144. The method of claim 143, wherein the comparative specimens comprise different stages in progression of a biologically dynamic tissue, wherein the biologically dynamic tissue is uterine endometrial tissue.

145. A method of examining biological samples, comprising:
placing a plurality of elongated biological samples at identifiable positions in a substrate that is capable of being sectioned;
sectioning the substrate to provide a plurality of substantial copies of an array of the biological samples, with the samples at the identifiable positions;
identifying one or more reference copies;
disseminating one or more dissemination copies to others; and
comparing a biological interpretation of one or more dissemination copies to a biological interpretation of one or more reference copies.

146. The method of claim 145, wherein the reference copies are included with a test kit.

147. The method of claim 145, wherein the biological interpretations of one or more dissemination copies are combined to provide a composite reference copy interpretation.

148. The method of claim 145, wherein disseminating the one or more reference copies comprises disseminating electronically.

149. The method of claim 145, wherein the biological samples comprise a library of multiple tissue samples.

150. A method of making a library of tissue specimens, comprising:
placing a plurality of elongated tissue samples of tissue specimens at identifiable positions in a substrate that is capable of being sectioned;
sectioning the substrate to provide a plurality of substantial copies of an array of the tissue samples, with the samples at the identifiable positions in the array.

151. The method of claim 150, further comprising associating an identifier with each identifiable position in the array.

152. The method of claim 151, wherein the identifier is and electronic identifier.

153. The method of claim 152, further comprising an electronic copy of the array.

154. The method of claim 153, further comprising an electronic identifier associated with one or more identifiable locations in the array.

155. A method for reviewing biological specimens, comprising:
providing multiple sections each comprising multiple biological samples;
obtaining images of the different sections after exposing the sections to the different biological reagents; and
disseminating the images to different recipients.

156. The method of claim 155, wherein the different recipients indicate an interpretation of the images, and communicate the interpretation to different recipients or a central source.

157. The method of claim 156, further comprising exposing different sections to different biological reagents that react with biological substrates of interest in the biological samples, and wherein the different recipients analyze the images to determine whether a reaction with a substrate has occurred in the different specimens.

158. The method of claim 157, further comprising obtaining information about subjects from whom the biological specimens were obtained, and correlating that information with the interpretation of the images.

159. The method of claim 155, wherein the biological samples are samples from tissue specimens, and there are at least 100 different tissue specimens present in each different section.

160. The method of claim 159, wherein the different tissue specimens are exposed to at least 100 different reagents.

161. The method of claim 159, wherein the biological samples are samples from at least 100 different tissue specimens in each different section.

162. The method of claim 161, wherein the different tissue specimens are exposed to at least 100 different reagents.

163. The method of claim 155, wherein obtaining images comprises obtaining digital images and storing the digital images.

164. The method of claim 155, wherein analyzing the images further comprises quantifying the reaction with the substrate.

165. The method of claim 155, wherein providing the plurality of sections comprises obtaining multiple elongated biological samples from multiple biological specimens, fixing the elongated biological samples substantially parallel to one another in a substrate, and sectioning the substrate substantially transverse to the elongated biological samples.

166. The method of claim 165, wherein providing multiple sections comprises:
punching a plurality of elongated receptacles in a recipient block;
punching elongated tissue specimen samples from the donor blocks, and placing tissue specimen samples in the receptacles in the recipient block; and
sectioning the recipient block substantially transverse to the elongated tissue specimen samples.

167. The method of claim 166, further comprising storing recipient block tissue identity information identifying tissue specimens contained within receptacles in recipient blocks, and recipient block location information defining the recipient block in a recipient block array.

168. The method of claim 110, wherein the results of the biological analyses are used to perform one or more or:
a. evaluating a reagent for disease diagnosis or treatment;
b. identifying a prognostic marker for cancer;
c. assessing or selecting therapy for a subject; or
d. finding a biochemical target for medical therapy.

169. The method of claim 110, wherein the biological sample is a tumor sample.

170. The method of claim 110, wherein the biological sample is a hematological or cytological preparation of cells.

171. A method for standardizing pathological evaluations, comprising:
visualizing a cellular specimen at a specific location in a cross-section of an microarray of a plurality of cellular specimens, wherein the array comprises a plurality of cellular specimens in a matrix, with the cellular specimens positioned at predetermined known positions in the matrix, such than when multiple sections of the matrix are provided, a two dimensional microarray of specimens is obtained, with each specimen at a predetermined position in the microarray;
analyzing the cellular specimen at the specific location to produce an evaluation of a particular biological characteristic; and
comparing the evaluation to a standard.

172. The method of claim 171, wherein the visualization is a computer generated image.

173. A method for training a person in histological analyses, comprising providing a section of a microarray of a plurality of cellular specimens for the person to evaluate, wherein the microarray comprises a plurality of cellular specimens in a matrix, with the cellular specimens positioned at predetermined known positions in the matrix, such than when multiple sections of the matrix are provided, a two dimensional microarray of specimens is provided, with each specimen at a predetermined position in the microarray, and a set of tissue-specific information for a cellular specimen in the microarray; and
comparing the evaluation of the person with a set of tissue-specific information.

174. The method of claim 173, wherein said set of tissue specific information is maintained at a remote location, and wherein said comparing is provided through an information network.

175. A method for parallel evaluation of tissue, comprising:
(a) displaying a computer generated image of a tissue specimen in a microarray of a plurality of cellular specimens of interest;
(b) producing an evaluation of the image for a clinical parameter, and
(c) comparing the evaluation to an analysis in a database.

153. The method of claim 152, further comprising
(d) displaying a further computer generated image of an additional cellular specimen in a microarray of a plurality of tissue specimens of interest;
(e) producing a further evaluation of the second image for a clinical parameter; and
(f) comparing the further evaluation to an analysis in a database.

176. The method of claim 175, further comprising:
repeating steps (d)-(f), until all of the cellular specimens in the microarray have been evaluated.

177. The method of claim 175, further comprising transmitting the evaluation to a remote location.

178. The method of claim 175, further comprising receiving feedback about the evaluation from a remote location.

179. A method for parallel evaluation of a cross-section of a cellular specimen, comprising:
(a) visualizing a first cross-section of the cellular specimen in a microarray of a plurality of cellular specimens of interest at a work-site, wherein the microarray comprises a plurality of cellular specimens in a matrix, with the cellular specimens positioned at predetermined known positions in the matrix, such than when multiple sections of the matrix are provided, a two dimensional microarray of specimens is provided, with each specimen at a predetermined position in the microarray, and wherein an immunological analysis, a histological stain, or a nucleic acid hybridization has been performed on each cellular specimen;
(b) analyzing a cross-section of the cellular specimen by examining the results of the immunological analysis, the histological stain, or the nucleic acid hybridization in the array to produce an evaluation of the cellular specimen for a clinical parameter; and
(c) comparing the evaluation of the cellular specimen to a standard evaluation in a data set comprising an evaluation of each of the cellular specimens of interest positioned at the predetermined known position in the microarray, wherein the data in the data set is accessible by position, and the data set is stored at the work-site or at a remote location.

180. A method for parallel evaluation of a cross-section of a cellular specimen, comprising:
(a) visualizing a first cross-section of the cellular specimen in a microarray of a plurality of cellular specimens of interest, wherein the microarray comprises a plurality of cellular specimens in a matrix, with the cellular specimens positioned at predetermined known positions in the matrix, such than when multiple sections of the matrix are provided, a two dimensional microarray of specimens is provided, with each specimen at a predetermined position in the microarray, and wherein an biological analysis comprising an immunological analysis, a histological stain, or a nucleic acid hybridization has been performed on each cellular specimen, wherein the cellular specimens has been produced in a first location;
(b) analyzing the first cross-section of the cellular specimen by examining the results of the immunological analysis, the histological stain, or the nucleic acid hybridization in the first cellular specimen to produce a first evaluation of the first cellular specimen for a clinical parameter;
(c) visualizing a second cross-section of a second cellular specimen in the microarray of a plurality of cellular specimens of interest, wherein the second cellular specimen has been produced in a second location distinct from the first location;
(d) analyzing the second cross-section of the cellular specimen by examining the results of the immunological analysis, the histological stain, or the nucleic acid hybridization in the second cellular specimen to produce a second evaluation of the cellular specimen for a clinical parameter;
(e) comparing the first evaluation of the first cellular specimen with the second evaluation of the second specimen, in order to compare the biological analysis performed on the first cellular specimen with the biological analysis performed on the second cellular specimen.
